(19) United States
(12) Patent Application Publication
(10) Pub. No.: US 2026/0098003 A1
(43) Pub. Date: Apr. 9, 2026

(54) INTEGRATED PROCESSES FOR THE PRODUCTION OF ISOBUTYLENE, POLYISOBUTYLENE, AND HIGHLY REACTIVE POLYISOBUTYLENE FROM ETHYLENE AND CRUDE C4 STREAMS

(71) Applicant: NTP Tec, LLC, Boerne, TX (US)

(72) Inventors: Clyde Edward BAXTER, Jr., League City, TX (US); Mark ROLL, Boerne, TX (US); Billy W. WAYCASTER, Cypress, TX (US)

(21) Appl. No.: 19/414,220

(22) Filed: Dec. 9, 2025

Related U.S. Application Data
(63) Continuation of application No. 19/352,125, filed on Oct. 7, 2025.
(60) Provisional application No. 63/705,024, filed on Oct. 8, 2024.

(51) Int. Cl. C07C 2/06; C08F 10/10

(52) U.S. Cl. CPC C07C 2/06; C08F 10/10

(57) ABSTRACT

Embodiments described herein generally relate to new processes and systems for producing isobutylene. Embodiments of the present disclosure also generally relate to new processes and systems for producing PIB, such as HR-PIB. Integrated processes for producing isobutylene from ethylene and/or crude C4 (CC4) streams, which may then be converted to PIB, are also provided. In an embodiment, a process for producing isobutylene is provided. The process includes dimerizing ethylene to produce a dimerization product effluent comprising a normal butylene. The process further includes skeletal isomerizing at least a portion of the normal butylene present in the dimerization product effluent to form a skeletal isomerization product effluent comprising isobutylene. The process further includes isomerizing at least a portion of 1-butene present in the skeletal isomerization product effluent to form an isomerization product effluent comprising 2-butene. The process further includes separating isobutylene and 2-butene from the isomerization product effluent.

### CROSS-REFERENCE TO RELATED APPLICATIONS

This application is a continuation of U.S. application Ser. No. 19/352,125, filed on Oct. 7, 2025, which claims benefit of and priority to U.S. Provisional Application Ser. No. 63/705,024, filed on Oct. 8, 2024. Each of these applications is incorporated herein by reference in its entirety.

### FIELD

Embodiments of the present disclosure generally relate to new processes and systems for producing isobutylene. Embodiments of the present disclosure also generally relate to new processes and systems for producing polyisobutylene (PIB), such as highly reactive polyisobutylene (HR-PIB).

### BACKGROUND

Isobutylene is a component utilized in the production of polymers used in various applications such fuel additives, lubricant additives, and rubber. High purity isobutylene ensures quality and consistency of end products that are made from it.

Traditional methods of making isobutylene used to produce PIB include dehydrogenation of isobutane, back-cracking of isobutylene ethers such as MTBE, dehydration of tert-butyl alcohol produced as a byproduct in propylene oxide production, or using the isobutylene present in raffinate-1 petrochemical streams. Such state-of-the-art methods, however, produce alcohol impurities and waste. Beyond these challenges, refineries are utilizing raffinate-1 streams in other processes. For example, instead of cracking naphtha (which has been the source of raffinate-1), the advent of cracking abundant cheap ethane to produce ethylene has led to large reductions in the available volume of raffinate-1 and the raffinate-1 contains little or no isobutylene.

One application of isobutylene is in the production of PIB. In the manufacture of conventional polyisobutylene (cPIB), state-of-the-art technologies directly polymerize the isobutylene in the raffinate-1 stream without separation of other butylene isomers also contained in the raffinate-1 stream. In the production of HR-PIB, however, conventional methods require the isobutylene to be of relatively high purity, especially with respect to other isobutylene isomers present in the feed stream to avoid undesirable products. For example, 1-butene and 2-butene in the feed stream act as chain terminators in the polymerization reaction resulting in polymers with less than the desired amount of the high reactivity vinylidene isomers to be considered HR-PIB. In addition, traditional technologies eliminate 1,3-butadiene from feeds for PIB production as 1,3-butadiene interferes with polymerization.

There is a need for new processes and systems for producing isobutylene. There is also a need for new processes and systems for producing PIB, such as HR-PIB.

### SUMMARY

Embodiments described herein generally relate to new processes and systems for producing isobutylene. Embodiments described herein also generally relate to new processes and systems for producing PIB, such as HR-PIB. Also provided is the integrated production of isobutylene and PIB, such as HR-PIB. In a first embodiment, ethylene may be dimerized and isomerized to produce isobutylene, which may then be polymerized into PIB. In a second embodiment is provided a unique combination of ethylene dimerization with conversion of a crude C4 stream to increase isobutylene production. In both embodiments, the isobutylene may be polymerized in, for example, a tubular loop reactor with a solid boron trifluoride (BF₃) catalyst complex, such as a BF₃-methanol (BF₃·MeOH) catalyst complex, which may help facilitate high efficiency and selectivity in producing HR-PIB suitable for various industrial applications such as fuel and lubricant additives, among other applications.

In an embodiment, a process for producing isobutylene is provided. The process includes dimerizing ethylene to produce a dimerization product effluent comprising a normal butylene. The process further includes skeletal isomerizing at least a portion of the normal butylene present in the dimerization product effluent to form a skeletal isomerization product effluent comprising isobutylene. The process further includes isomerizing at least a portion of 1-butene present in the skeletal isomerization product effluent to form an isomerization product effluent comprising 2-butene. The process further includes separating isobutylene and 2-butene from the isomerization product effluent.

In another embodiment, a process for producing isobutylene is provided. The process includes dimerizing ethylene to produce a dimerization product effluent comprising a normal butylene. The process further includes skeletal isomerizing at least a portion of the normal butylene present in the dimerization product effluent to form a skeletal isomerization product effluent comprising isobutylene. The process further includes isomerizing at least a portion of 1-butene present in the skeletal isomerization product effluent to form an isomerization product effluent comprising 2-butene.

In another embodiment, a process for producing PIB is provided. The process includes dimerizing ethylene to produce a dimerization product effluent comprising a normal butylene. The process further includes skeletal isomerizing at least a portion of the normal butylene present in the dimerization product effluent to form a skeletal isomerization product effluent comprising isobutylene. The process further includes isomerizing at least a portion of 1-butene present in the skeletal isomerization product effluent to form an isomerization product effluent comprising 2-butene. The process further includes separating isobutylene and 2-butene from the isomerization product effluent. The process further includes polymerizing the isobutylene separated from the isomerization product effluent into a polymerization product effluent comprising PIB.

In another embodiment, a system is provided. The system includes an olefin-producing unit. The system further includes an ethylene dimerization reactor coupled to and downstream from the olefin-producing unit. The system further includes a skeletal isomerization reactor coupled to and downstream from the ethylene dimerization reactor. The system further includes an isomerization reactor coupled to and downstream from the skeletal isomerization reactor. The system further includes a separation unit coupled to and downstream from the isomerization reactor. The system further includes a line coupling the separation unit to the skeletal isomerization reactor, the line configured to recycle or feed 2-butene from the separation unit to the skeletal isomerization reactor.

In another embodiment, a process for producing isobutylene from ethylene and CC4 is provided. The process includes dimerizing ethylene to produce a dimerization product effluent comprising 1-butene, 2-butene, or combinations thereof. The process further includes feeding CC4 and the dimerized product effluent into a selective hydrogenation-isomerization unit, wherein the CC4 comprises 1,3-butadiene. The process further includes selectively hydrogenating and isomerizing the 1,3-butadiene in the SHU to form a selective hydrogenation-isomerization product effluent comprising 2-butene. The process further includes separating isobutylene and the 2-butene from the selective hydrogenation-isomerization product effluent. The process further includes skeletal isomerizing at least a portion of the 2-butene separated to form a skeletal isomerization product effluent comprising isobutylene. The process further includes recycling the skeletal isomerization product effluent to the SHU. The process further includes converting, in the SHU, 1-butene present in the skeletal isomerization product effluent to 2-butene.

In another embodiment, a process for producing PIB is provided. The process includes dimerizing ethylene to produce a dimerization product effluent comprising 1-butene, 2-butene, or combinations thereof. The process further includes feeding CC4 and the dimerized product effluent into a selective hydrogenation-isomerization unit. The process further includes selectively hydrogenating and isomerizing 1,3-butadiene in the SHU to form a selective hydrogenation-isomerization product effluent comprising 2-butene. The process further includes separating isobutylene and the 2-butene from the selective hydrogenation-isomerization product effluent. The process further includes skeletal isomerizing at least a portion of the 2-butene separated to form a skeletal isomerization product effluent comprising isobutylene. The process further includes recycling the skeletal isomerization product effluent to the SHU. The process further includes converting, in the SHU, 1-butene present in the skeletal isomerization product effluent to 2-butene. The process further includes polymerizing the isobutylene separated from the selective hydrogenation-isomerization product effluent into a polymerization product effluent comprising PIB.

In another embodiment, a system is provided. The system includes an olefin-producing unit. The system further includes an ethylene dimerization reactor coupled to and downstream from the olefin-producing unit. The system further includes a selective hydrogenation-isomerization unit coupled to and downstream from the ethylene dimerization reactor, the selective hydrogenation-isomerization unit further coupled to and downstream from the olefin-producing unit. The system further includes a separation unit coupled to and downstream from the selective hydrogenation-isomerization unit. The system further includes a skeletal isomerization reactor coupled to and downstream from the separation unit. The system further includes a line coupling the skeletal isomerization reactor to the selective hydrogenation-isomerization unit.

### BRIEF DESCRIPTION OF THE DRAWINGS

So that the manner in which the above recited features of the present disclosure may be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limited of its scope, and may admit to other equally effective embodiments.

FIG. 1 is a generalized schematic flow diagram showing various implementations of a system for forming isobutylene and PIB described herein.

FIG. 2A shows selected operations of a process for forming isobutylene according to at least one embodiment of the present disclosure.

FIG. 2B shows selected operations of a process for forming PIB according to at least one embodiment of the present disclosure.

FIG. 3 is a generalized schematic flow diagram showing various implementations of a system for forming isobutylene and PIB described herein.

FIG. 4A shows selected operations of a process for forming isobutylene according to at least one embodiment of the present disclosure.

FIG. 4B shows selected operations of a process for forming PIB according to at least one embodiment of the present disclosure.

### DETAILED DESCRIPTION

Embodiments of the present disclosure generally relate to new processes and systems (or processing plants) for producing isobutylene. The inventors found novel processes and systems to convert ethylene to isobutylene. The inventors also found novel processes and systems to uniquely integrate ethylene dimerization with the conversion of a crude C4 (CC4) stream from a stream cracker (or other olefin-producing unit). Here, the CC4 stream may be processed alongside a dimerized ethylene stream to enhance and/or increase isobutylene production. Isobutylene produced by embodiments described herein may include high purity isobutylene. Advantageously, embodiments described herein may allow for all, or nearly all of, butylenes present in an ethylene dimerization product effluent, a CC4 stream, or combinations thereof, among other C4-containing feeds, to be converted into isobutylene. Isobutylene produced by embodiments described herein may be fed to a polymerization reactor for forming PIB, such as HR-PIB.

The term “normal butylenes”, also referred to as linear butenes and linear butylenes, includes 1-butene and 2-butene(s) (for example, cis-2-butene and/or trans-2-butene). Reference to 2-butene includes reference to both cis-2-butene and trans-2-butene unless specified to the contrary or the context clearly indicates otherwise. Isobutylene is a branched butene.

The terms “butylenes” and “butenes” includes 1-butene, 2-butene, and isobutylene.

The terms “butadiene”, “1,3-butadiene”, and “1,2-butadiene” are used interchangeably herein unless specified to the contrary or the context clearly indicates otherwise.

Embodiments of the present disclosure also generally relate to new processes and systems (processing plants) for forming PIB and HR-PIB. The inventors found a novel processing scheme that may be used to advantageously convert all, or nearly all, isobutylene to PIB, for example, HR-PIB. The conversion of isobutylene to PIB, such as HR-PIB, may be integrated with the isobutylene production process such that all or nearly all of the butylenes (for example, normal butylenes and isobutylene) in a feedstock are converted to PIB, such as HR-PIB.

In addition, embodiments described herein may facilitate circular economy (sustainable) implementations, for example, by recycling isobutylene oligomer coproducts, minimizing waste, and integrating hydrotreating or cracking steps.

Conventional approaches to forming isobutylene lack efficiency and high conversion. For example, conventional approaches fail to include an isomerization operation converting 1-butene to 2-butene prior to separation. Without this isomerization, 1-butene and isobutylene are not efficiently separated due to the narrow boiling point gap between the 1-butene and isobutylene (less than 1° C.). In contrast, embodiments described herein may include an isomerization operation to convert 1-butene to 2-butene prior to separation, which may enable for more efficient recovery of isobutylene and a more pure isobutylene fraction. High purity isobutylene is desirable not only for HR-PIB production, but also for fuel additives, lubricant additives, and rubber.

In addition, conventional technologies fail to integrate with CC4 streams and fail to utilize 1,3-butadiene. In contrast, embodiments of the present disclosure may include integration of CC4 streams to make isobutylene with streams produced from ethylene dimerization. Further, embodiments described herein may enable usage of 1,3-butadiene by inclusion of, for example, a selective hydrogenation-isomerization operation which converts 1,3-butadiene into 2-butene. This may facilitate high utilization and conversion of C4 olefins (including 1,3-butadiene) to isobutylene not observed in conventional approaches to isobutylene.

Conventional approaches to forming PIB, including HR-PIB, are also deficient. In contrast, and in some examples, isobutylene may be polymerized in a tubular loop reactor operating at short residence times (for example, 4 minutes or less) and at high circulation velocities, using a sorbed BF₃·MeOH catalyst that avoids β-hydrogen elimination. This reactor/catalyst system may achieve greater than 80-90% alpha vinylidene content and narrow molecular weight distributions, outcomes not achievable with conventional polymerization approaches. In further contrast to conventional approaches for forming PIB, integration of PIB production processes and systems with isobutylene production processes and systems provide as described herein may facilitate high utilization and conversion of C4 olefins to PIB, such as HR-PIB.

Moreover, embodiments of the of the present disclosure may include one or more of: feedstock integration of ethylene with CC4 streams, selective hydrogenation-isomerization of butadiene and 1-butene, a full selective hydrogenation-isomerization-separation-skeletal isomerization recycle loop, and polymerization to HR-PIB. The selective hydrogenation-isomerization-separation-skeletal isomerization recycle loop is also referred to herein as SHU-Fractionation-SKIP loop.

In addition, embodiments described herein may enable optional downstream flexibility through, for example, hydrogenation of HR-PIB and an alternative metathesis pathway, which may be utilized to convert 2-butene and ethylene into propylene, thereby further improving process integration and carbon efficiency.

In further contrast to state-of-the-art technologies, embodiments described herein may include an integrated ethylene to isobutylene process that includes dimerization, isomerization, separation, and skeletal isomerization. In addition, and in further contrast to conventional technologies, embodiments described herein may include an ethylene to isobutylene process that is utilized with the integrated process of converting steam cracker CC4 streams to isobutylene. Such integration with steam cracker CC4 streams may provide certain advantages, such as low cost and high yield potential. CC4 streams are low cost, while ethylene dimerization provides a higher proportion of C4 olefins that are converted to isobutylene. In addition, both the CC4 feed and the feed for ethylene dimerization may be sourced from the same olefin-producing unit (for example, the same steam cracker).

The use of headings is for purposes of convenience only and does not limit the scope of the present disclosure. Embodiments described herein may be combined with other embodiments.

HR-PIB is a composition that includes greater than 75% alpha vinylidene olefin isomer. PIB having large amounts of alpha vinylidene olefin content are termed HR-PIB because the reactivity in the derivative reactions, particularly to make fuel and lubricant additives, is greatly enhanced.

HR-PIB compositions described herein may contain additional olefin isomers including beta vinylidene olefin isomer, other trisubstituted olefin isomers, internal vinylidenes, and tetrasubstituted olefin isomers. As used herein, a “composition” may include component(s) of the composition, reaction product(s) of two or more components of the composition, a remainder balance of remaining starting component(s), or combinations thereof. HR-PIB is termed highly reactive because of its increased reactivity in derivatization reactions, such as reactions with maleic anhydride to produce polyisobutenylsuccinic anhydride (PIBSA) to form precursors useful for fuel and lubricant additives.

Conventional polyisobutylene (cPIB), in contrast to HR-PIB, has a majority of its olefin isomers other than alpha vinylidene. cPIB typically has 10% or less of alpha vinylidene olefin isomer and is therefore significantly less reactive in derivatization reactions than HR-PIB.

The alpha vinylidene olefin isomer (also referred to as α-vinylidene) of a PIB composition (or HR-PIB composition) may be represented by formula (IA):

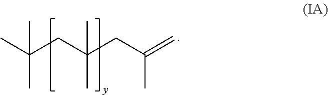

As shown in formula (IA), alpha vinylidene has the double bond (or olefin) positioned in the terminal position of the molecule, allowing it to react more quickly when producing derivatives. As described herein, HR-PIB includes greater than 75% alpha vinylidene olefin isomer whereas cPIB has about 10% or less of alpha vinylidene olefin isomer.

The beta vinylidene olefin isomer (also referred to as β-vinylidene) of a PIB composition (or HR-PIB composition) may be represented by formula (IB):

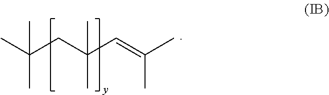

An internal disubstituted vinylidene olefin isomer of a PIB composition (or HR-PIB composition) may include the following structure represented by formula (IC):

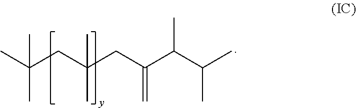

Other internal vinylidenes are possible, including where the position of the olefin in the polyisobutylene is such that the olefin is disubstituted and not at the end of the carbon chain. Other trisubstituted olefin isomers and tetrasubstituted olefin isomers may be produced in the polymerizations described herein.

Processes described herein may be used to convert an amount of C4 olefins that are not isobutylene (for example, butadiene, 1-butene, cis-2-butene, trans-2-butene, or combinations thereof) in a C4-containing feed to isobutylene. The conversion of C4 olefins that are not isobutylene to isobutylene by processes described herein may be about 70% or more, about 80% or more, about 90% or more, about 95% or more, about 96% or more, about 97% or more, about 98% or more, about 99% or more, or about 100% based on a total amount of C4 olefins that are not isobutylene in the C4-containing feed used for the conversion to isobutylene. The total amount of C4 olefins that are not isobutylene in the C4-containing feed used for the conversion to isobutylene is based on the total amount of butadiene, 1-butene, cis-2-butene, trans-2-butene, or combinations thereof in the C4-containing feed used for forming isobutylene. The conversion of C4 olefins that are not isobutylene to isobutylene may be such that all, or essentially all, of the C4 olefins that are not isobutylene in the C4-containing feed are converted to isobutylene. That is, processes described herein may be used to make high purity isobutylene.

Processes described herein may be used to convert an amount of C4 olefins (isobutylene, butadiene, 1-butene, cis-2-butene, trans-2-butene, or combinations thereof) in a C4-containing feed to PIB, such as HR-PIB. The conversion of C4 olefins in a C4-containing feed to PIB (for example, HR-PIB) by processes described herein may be about 70% or more, about 80% or more, about 90% or more, about 95% or more, about 96% or more, about 97% or more, about 98% or more, about 99% or more, or about 100% based on a total amount of C4 olefins in the C4-containing feed used for the conversion to HR-PIB. The total amount of C4 olefins in the C4-containing feed used for the conversion to PIB (e.g., HR-PIB) is based on the total amount of isobutylene, butadiene, 1-butene, cis-2-butene, trans-2-butene, or combinations thereof in the C4-containing feed for forming the PIB (e.g., HR-PIB). The conversion of C4 olefins to isobutylene may be such that all, or essentially all, of the C4 olefins in the C4-containing feed are converted to PIB (e.g., HR-PIB). That is, processes described herein may be used for efficient and cost-effective production of PIB (e.g., HR-PIB).

C4-containing feeds converted by processes described herein may include a dimerization product effluent (e.g., an effluent from an ethylene dimerization reactor), an effluent from an olefin-producing unit (such as a steam cracker and/or a dehydration unit), a CC4 stream, an effluent from n-butane dehydroisomerization to isobutylene, an effluent from isobutane dehydrogenation to isobutylene, or combinations thereof. These effluents are C4-containing feeds comprising C4 olefins such as butadiene, 1-butene, cis-2-butene, trans-2-butene, or combinations thereof, and optionally isobutylene. The effluent from n-butane dehydrogenation may include 1-butene, 2-butene, or combinations thereof. If so, the effluent from the n-butane dehydrogenation may be sent to a skeletal isomerization reactor as described herein.

Any suitable C4-containing feed that includes a C4-hydrocarbon may be utilized. A C4 hydrocarbon may include a C4 olefin and optionally a C4 alkane. A C4 olefin is a hydrocarbon containing 4 carbon atoms and at least one carbon-carbon double bond (olefin). C4 olefins may include 1,3-butadiene, normal butylenes (also referred to as n-butylenes), isobutylene, or combinations thereof. C4 alkanes may include butane, such as normal butane (also referred to as n-butane), isobutane (also referred to as i-butane and 2-methylpropoane), or combinations thereof.

C4-containing feeds may include an effluent obtained from the cracking of hydrocarbons such as naphtha, gas oils, lighter hydrocarbons, or combinations thereof. C4-containing feeds may include a CC4 stream or effluent produced from an olefin-producing unit such as a steam cracker or a dehydrogenation reactor utilized to make light hydrocarbon olefins. Feeds to olefin-producing units may include light hydrocarbons (e.g., C2-C6 hydrocarbons, such as C2-C4 hydrocarbons, such as ethane, ethylene, propane, propylene, butane, butylenes, or combinations thereof), hexanes, naphtha, or combinations thereof.

CC4 streams may include 1,3-butadiene, 1-butene, cis-2-butene, trans-2-butene, or combinations thereof. CC4 streams may optionally include isobutylene, such as a minor amount of isobutylene. CC4 streams may optionally include butane. CC4 streams may be sourced from an olefin-producing unit (such as a steam cracker), among other sources. CC4 streams may also be referred to herein as CC4 feeds.

C4-containing feeds may include any suitable amount of normal butylenes (1-butene, cis-2-butene, trans-2-butene, or combinations thereof). A total amount of normal butylenes in the C4-containing feed may be greater than 0.1 wt %, such as in a range from about 0.1 wt % to about 99.9 wt %, such as from about 1 wt % to 99 wt %, such as from about 3 wt % to about 97 wt %, such as from about 5 wt % to about 95 wt %, such as from about 10 wt % to about 90 wt %, such as from about 15 wt % to about 85 wt %, such as from about 20 wt % to about 80 wt %, such as from about 25 wt % to about 75 wt %, such as from about 30 wt % to about 70 wt %, such as from about 35 wt % to about 65 wt %, such as from about 40 wt % to about 60 wt %, such as from about 45 wt % to about 55 wt %, based on the total wt % of the C4-containing feed.

The total wt % of a C4-containing feed is equal to 100 wt %. When using any suitable C4-containing feed, any unreacted portion of the C4-containing feed may be recycled by or through various parts of processing schemes described herein.

C4-containing feeds may include any suitable amount of isobutylene. An amount of isobutylene in a C4-containing feed may be about 1 wt % or less, such as about 0.5 wt %, such as about 0.1 wt % or less, such as about 0.05 wt % or less, such as about 0.01 wt % or less, such as 0 wt % or an undetectable amount based on the total wt % of the C4-containing feed. Additionally, or alternatively, an amount of isobutylene in a C4-containing feed may be greater than 0.1 wt %, such as in a range from about 0.1 wt % to about 99.9 wt %, such as from about 1 wt % to 99 wt %, such as from about 3 wt % to about 97 wt %, such as from about 5 wt % to about 95 wt %, such as from about 10 wt % to about 90 wt %, such as from about 15 wt % to about 85 wt %, such as from about 20 wt % to about 80 wt %, such as from about 25 wt % to about 75 wt %, such as from about 30 wt % to about 70 wt %, such as from about 35 wt % to about 65 wt %, such as from about 40 wt % to about 60 wt %, such as from about 45 wt % to about 55 wt %, or from about 10 wt % to about 45 wt %, such as from about 15 wt % to about 40 wt %, such as from about 20 wt % to about 35 wt %, such as from about 25 wt % to about 30 wt % based on the total wt % of the C4-containing feed. Additionally, or alternatively, an amount of isobutylene in the C4-containing feed may be about 5 wt % or less, such as from about 0 wt % to about 4 wt %, such as from about 0.1 wt % to about 2 wt %, such as from about 0.5 wt % to about 1 wt % based on the total wt % of the C4-containing feed.

C4-containing feeds may include any suitable amount of 1,3-butadiene. An amount of isobutylene in a C4-containing feed may be about 1 wt % or less, such as about 0.5 wt %, such as about 0.1 wt % or less, such as about 0.05 wt % or less, such as about 0.01 wt % or less, such as 0 wt % or an undetectable amount based on the total wt % of the C4-containing feed. Additionally, or alternatively, an amount of 1,3-butadiene in a C4-containing feed may be greater than 0.1 wt %, such as in a range from about 0.1 wt % to about 99.9 wt %, such as from about 1 wt % to 99 wt %, such as from about 3 wt % to about 97 wt %, such as from about 5 wt % to about 95 wt %, such as from about 10 wt % to about 90 wt %, such as from about 15 wt % to about 85 wt %, such as from about 20 wt % to about 80 wt %, such as from about 25 wt % to about 75 wt %, such as from about 30 wt % to about 70 wt %, such as from about 35 wt % to about 65 wt %, such as from about 40 wt % to about 60 wt %, such as from about 45 wt % to about 55 wt %, or from about 10 wt % to about 45 wt %, such as from about 15 wt % to about 40 wt %, such as from about 20 wt % to about 35 wt %, such as from about 25 wt % to about 30 wt %, based on the total wt % of the C4-containing feed. Additionally, or alternatively, an amount of 1,3-butadiene in the C4-containing feed may be about 5 wt % or less, such as from about 0 wt % to about 4 wt %, such as from about 0.1 wt % to about 2 wt %, such as from about 0.5 wt % to about 1 wt % based on the total wt % of the C4-containing feed.

Any suitable C4-containing feed, such as those described herein, may be used for processes for forming isobutylene, for processes for forming PIB, such as HR-PIB, or combinations thereof.

Although embodiments described herein may utilize crude C4-containing feeds and effluents from ethylene dimerization, any suitable C4-containing feed may be utilized. In addition, any suitable ethylene-containing feed may be utilized for ethylene dimerization. Although embodiments of the present disclosure may be described with respect to forming HR-PIB, embodiments described herein may be used for forming PIB unless specified to the contrary or the context clearly indicates otherwise.

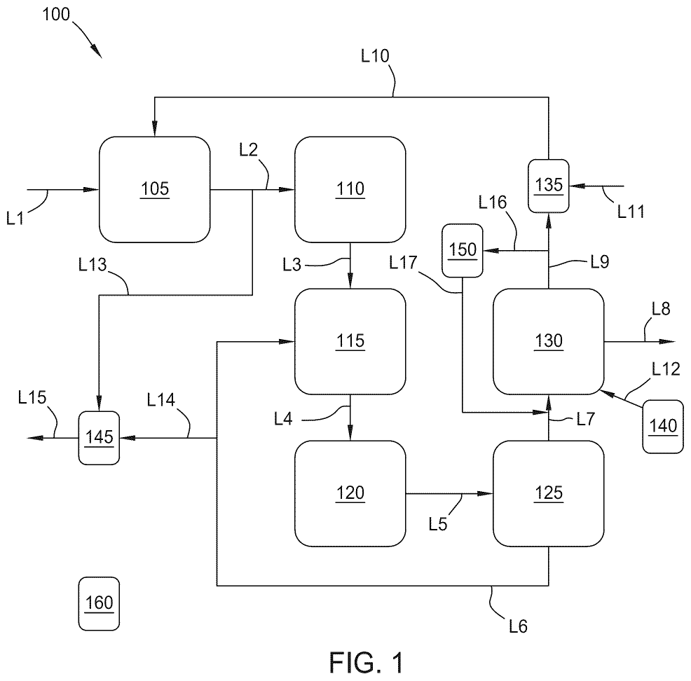

FIG. 1 is a generalized schematic flow diagram showing various embodiments of processes described herein corresponding to operational areas or units in a system 100 (for example, a processing plant) for forming isobutylene and PIB according to at least one embodiment of the present disclosure. The system 100 includes embodiments for isobutylene production and PIB (for example, HR-PIB) production. The system 100 may be run in a batch or a continuous process. In some embodiments, which may be combined with other embodiments, the system 100 may convert ethylene into isobutylene by two operations. For example, ethylene may be first dimerized to produce 1-butene and 2-butene, followed by skeletal isomerization to convert at least a portion of these linear butylenes into isobutylene. The isobutylene may then be separated and polymerized into PIB, such as HR-PIB.

A light hydrocarbon (LHC) stream may enter the system 100 through line L1 and enters an olefin-producing unit 105 such as a steam cracker or a dehydrogenation reactor. The light hydrocarbon stream may include a C2-C4 hydrocarbon stream such as ethane, propane, butane, or combinations thereof, such as ethane. The olefin-producing unit 105 converts light hydrocarbons present in the light hydrocarbon stream under olefin-producing conditions effective to form an olefin-containing effluent comprising ethylene, a CC4 feed, or combinations thereof. Additionally, or alternatively, an ethylene stream may enter the system 100 through line L2 and enter the ethylene dimerization reactor 110 such that the olefin-producing unit 105 is optional.

The conversion of light hydrocarbons into an olefin-containing effluent comprising ethylene in olefin-producing unit 105 is performed under any suitable olefin-producing conditions effective to convert the light hydrocarbon stream to olefin-containing effluent that includes ethylene. In a representative steam cracker, suitable olefin-producing conditions at olefin-producing unit 105 may include a temperature in a range from about 750° C. to about 900° C., such as from about 850° C. to about 875° C. Suitable olefin-producing conditions at olefin-producing unit 105 may include a pressure in a range from about 175 kPa to about 240 kPa, such as from about 200 kPa to about 225 kPa. A residence time may be from about 0.1 seconds to about 2 seconds, such as from about 0.1 seconds to about 0.5 seconds, such as from about 0.2 seconds to about 0.4 seconds. A dilution steam-to-hydrocarbon ratio may be in a range from about 0.1 kg steam/kg feed to about 1 kg steam/kg feed, such as from about 0.2 kg steam/kg feed to about kg steam/kg feed. Where desired, a gas hourly space velocity may be inferred from the coil volume and residence time.

The olefin-containing effluent comprising ethylene may also include CC4. In ethane cracking, the CC4 stream typically constitutes from about 2 wt % to about 5 wt % of the total cracked product and contains about 80 wt % total C4 olefins, which may include about 70 wt % 1,3-butadiene along with 1-butene and 2-butene. The CC4 may contain little or no isobutylene or isobutane. Ethylene and CC4 may be separated by any suitable separation technique. For example, suitable separation techniques may include performing a distillation, a fractional distillation, a vacuum distillation, a flash evaporation, a fractionation, an extraction, a decantation, a coalescence, or combinations thereof, on the olefin-containing effluent. This separation may be performed at the olefin-producing unit 105.

Additionally, or alternatively, ethylene used for the ethylene dimerization (described below) may be produced from various sources, including petroleum-based, fossil fuel-based, bio-based materials, or combinations thereof. For example, with respect to bio-based materials, ethylene may be derived from ethanol through a dehydration process using alumina or other solid acid catalysts at a temperature in a range from about 300° C. to about 400° C. Petroleum-based ethylene may be generated in refinery steam crackers, where light hydrocarbon streams, often derived from natural gas or oil refining byproducts, are cracked. Ethane is frequently used due to its high ethylene yield, availability, and cost-effectiveness. A cracking process of ethane may operate as described above.

The olefin-containing effluent comprising ethylene (also referred to herein as an ethylene stream) may exit the olefin-producing unit 105 through line L2 and may be flowed to an ethylene dimerization reactor 110. The ethylene dimerization reactor 110 may include any suitable reactor such as a batch reactor, a continuous flow reactor, a tank reactor, a tubular reactor, a tubular loop reactor, a continuous stirred tank reactor, a plug flow reactor, a fluidized bed reactor, an immobilized bed reactor, a fixed bed reactor, or combinations thereof, such as such as a continuous flow reactor, a continuous stirred tank reactor, a tubular reactor, a tubular loop reactor, or combinations thereof. At the ethylene dimerization reactor 110, ethylene is converted, under dimerization conditions, to a dimerization product effluent. The dimerization product effluent (a C4-containing feed) may include a normal butylene, for example, 1-butene, cis-2-butene, trans-2-butene, or combinations thereof. The dimerization product effluent may optionally include isobutylene. The dimerization may be performed with use of any suitable dimerization catalyst. Dimerization catalysts may include a nickel-based catalyst, such as a nickel organometallic complex, a nickel metallocene, a nickel-zeolite, a nickel oxide supported on alumina or silica, a nickel sulfide supported on a alumina or silica, or combinations thereof. At the ethylene dimerization reactor 110, a mixture comprising the ethylene and the catalyst may be reacted under dimerization conditions to form the dimerization product effluent. Here, the ethylene may contact the dimerization catalyst under the dimerization conditions. Dimerization conditions may include a temperature in a range from about 50° C. to about 150° C., such as from about 75° C. to about 125° C., such as from about 95° C. to about 105° C. Dimerization conditions may include a pressure in a range from about 1 MPa to about 3 MPa, such as from about 1 MPa to about 2 MPA or from about 1.5 MPa to about 2.5 MPa, such as from about 1.75 MPa to about 2.25 MPa.

The dimerization product effluent may exit the ethylene dimerization reactor 110 through line L3 and may be flowed to a skeletal isomerization reactor 115 (also referred to as a SKIP reactor). The skeletal isomerization reactor 115 may be operated to perform a skeletal isomerization process (SKIP process). At the skeletal isomerization reactor 115, at least a portion of the normal butylene (1-butene, trans-2-butene, cis-2-butene, or combinations thereof) may be skeletally isomerized, under skeletal isomerization conditions, to form a skeletal isomerization product effluent that is enriched in isobutylene.

As used herein, an effluent that is “enriched” in, for example, isobutylene, refers to an effluent where the relative amount (or concentration) of isobutylene in an effluent after a process operation (for example, after a skeletal isomerization process) is greater than the relative amount (or concentration) of isobutylene in the effluent before the process operation. For example, if an effluent includes 0.1 wt % isobutylene before a process operation (for example, a skeletal isomerization process), the effluent formed after the process operation (for example, the skeletal isomerization process) would include greater than 0.1 wt % isobutylene.

At high temperatures and in the presence of a catalyst, the normal butylenes (1-butene and 2-butene) and isobutylene may reach a chemical equilibrium such that the amount of isobutylene may be increased or maximized. The skeletal isomerization product effluent exiting the skeletal isomerization reactor 115 through line L4 may optionally include one or more normal butylenes. The skeletal isomerization may be performed with the use of any suitable skeletal isomerization catalyst. Suitable skeletal isomerization catalysts may include an acidic catalyst, such as silica-alumina. At the skeletal isomerization reactor 115, normal butylene (e.g., 1-butene, cis-2-butene, trans-2-butene, or combinations thereof) present in the dimerization product effluent react with the skeletal isomerization catalyst under skeletal isomerization conditions to form the skeletal isomerization product comprising isobutylene. Here, the 1-butene, cis-2-butene, trans-2-butene, or combinations thereof present in the dimerization product effluent may contact the skeletal isomerization catalyst to form isobutylene. Skeletal isomerization conditions may include a temperature from about 200° C. to about 500° C., such as from about 200° C. to about 400° C., such as from about 250° C. to about 350° C., such as from about 275° C. to about 325° C., or from about 450° C. to about 500° C. Skeletal isomerization conditions may include a pressure of about atmospheric pressure, and/or a LHSV from about 4 h⁻¹ to about 5 h⁻¹, such that the reaction is in the vapor phase. The butylenes vapors may be diluted with nitrogen at a weight ratio from about 1.4 to about 1.5. Selectivity to isobutylene may be greater than about 50%.

The feed entering the skeletal isomerization reactor 115 through line L3 may have a lower concentration of isobutylene than the concentration of isobutylene in the skeletal isomerization product effluent exiting the skeletal isomerization reactor 115 through line L4. The skeletal isomerization effluent exiting the skeletal isomerization reactor 115 through line L26 may include isobutylene, 2-butene, 1-butene, or combinations thereof.

The skeletal isomerization product effluent comprising isobutylene (e.g., enriched in isobutylene) may exit the skeletal isomerization reactor 115 through line L4 and may be flowed to an isomerization reactor 120. The isomerization reactor may include any suitable reactor such as a fixed bed reactor. At the isomerization reactor 120, residual 1-butene present in the skeletal isomerization effluent may be converted, under isomerization conditions, to 2-butene by isomerization. This conversion is useful for improving the efficiency of the subsequent separation of isobutylene from normal butylenes. The reason for this is that 2-butene has a significantly higher boiling point than isobutylene, while 1-butene has a very close boiling point to that of isobutylene. The boiling point of isobutylene is −6.9° C.; the boiling point of 1-butene is −6.3° C.; the boiling point of trans-2-butene is 0.8-0.9° C.; and the boiling point of cis-2-butene is 3.7° C. The boiling points indicate that separation of isobutylene from 1-butene is very difficult as the boiling point difference is less than 1° C. In contrast, the boiling point difference between isobutylene and 2-butene is much greater, with a boiling point difference of about 7° C. to about 11° C. Accordingly, conversion of 1-butene present in the skeletal isomerization product effluent to 2-butene is advantageous and facilitates more efficient downstream separation (for example, in separation unit 125).

The isomerization in the isomerization reactor 120 may be performed with the use of any suitable isomerization catalyst, such as an acidic catalyst. Suitable isomerization catalysts may include, for example: silica-alumina; alumina; a zeolite (such as H-mordenite and/or H-ZSM-5); a supported metal oxide (such as tungsten oxide and/or molybdenum oxide supported on alumina and/or silica); a chlorinated alumina; a metal-supported alumina (for example, platinum on alumina (Pt/Al₂O₃)); or combinations thereof. These catalysts may facilitate double-bond migration from 1-butene to 2-butene while minimizing cracking and oligomerization side reactions.

The 1-butene may contact the isomerization catalyst under isomerization conditions to form 2-butene. Suitable isomerization conditions may include a temperature in a range from about 100° C. to about 200° C., such as from about 125° C. to about 175° C., such as from about 140° C. to about 160° C. Suitable isomerization conditions may include a pressure in a range from about 0.5 MPa to about 1.5 MPa, such as from about 0.75 MPa to about 1.25 MPa, such as from about 0.9 MPa to about 1.1 MPa. Suitable isomerization conditions may include a liquid hourly space velocity (LHSV) may be in a range from about 0.5 h⁻¹ to about 5 h⁻¹, such as from about 1 h⁻¹ to about 3 h⁻¹, depending on, for example, feed composition and desired conversion.

The isomerization product effluent may include 2-butene, isobutylene, 1-butene, or combinations thereof. The isomerization product effluent is enriched in 2-butene. For example, a concentration of 2-butene present in the isomerization product effluent exiting the isomerization reactor 120 through line L5 may be greater than a concentration of 2-butene present in the skeletal isomerization product effluent entering the isomerization reactor 120 through line L4. Additionally, or alternatively, the isomerization product effluent exiting the isomerization reactor 120 through line L5 may have a concentration of 1-butene that is less than a concentration of 1-butene present in the skeletal isomerization product effluent entering the isomerization reactor 120 through line L4. Isobutylene that enters the isomerization reactor 120 is generally unchanged under the isomerization conditions. The isomerization product effluent comprising 2-butene and isobutylene may exit the isomerization reactor 120 through line L5 and may be flowed to a separation unit 125.

In some embodiments, which may be combined with other embodiments, the skeletal isomerization reactor 115 and the isomerization reactor may be a single combination reactor. Here, the isomerization of 1-butene to 2-butene and the skeletal isomerization of 2-butene to isobutylene may be performed in the single combination reactor, such that, e.g., a 1-butene stream entering the single combination reactor exits as an effluent comprising isobutylene and 2-butene, with little-to-no 1-butene (or substantially free of 1-butene). Substantially free of 1-butene refers to an amount of 1-butene in the effluent exiting the single combination reactor that is less than 5 wt %, such as less than 3 wt %, such as less than 1 wt %, such as less than 0.5 wt %, such as less than 0.1 wt % 1-butene, or an undetectable amount based on a total wt % of the effluent exiting the single combination reactor, the total wt % of the effluent exiting the single combination reactor equal to 100 wt %. The single combination reactor may be operated under any suitable conditions, such as those described with respect to the skeletal isomerization conditions and/or the isomerization conditions. An acidic catalyst may be utilized in the single combination reactor, for example, silica-alumina or a different acidic catalyst such as those described herein for skeletal isomerization and isomerization. In these and other embodiments, the feed entering the single combination reactor may include the dimerization product effluent. The effluent exiting the single combination reactor may be flowed to the separation unit 125 and may be separated in the same, or a similar, manner as that described herein with respect to the separation of the isomerization product effluent.

At the separation unit 125, the components of the isomerization product effluent are separated. For example, the isomerization product effluent may be separated into an isobutylene fraction (which may exit the separation unit 125 through line L7) and a 2-butene fraction (which may exit the separation unit 125 through line L6). The isomerization product effluent may also include n-butane, isobutane, or combinations thereof. In such instances, the separation unit 125 may also separate an isobutane fraction and an n-butane fraction which may exit the separation unit 125 through different lines (not shown).

The separation unit 125 may include any suitable apparatus to separate the components (or fractions) from the isomerization product effluent, such as an apparatus operable to perform a distillation, a vacuum distillation, a fractional distillation, a reactive distillation, a flash evaporation, a fractionation, an extraction, a decantation, a coalescence, or combinations thereof. For example, the separation unit 125 may include a fractional distillation column that separates the components (or fractions) by boiling points.

The separation at separation unit 125 may be performed under any suitable conditions effective to separate the various components of the isomerization product effluent. For example, separation unit 125 may include a fractionation column or columns. The fractionation column(s) may be operated with a residence time that is from about 20 minutes to about 100 minutes, such as from about 25 minutes to about 75 minutes, such as from about 30 minutes to about 60 minutes at a column pressure that is from about 50 pounds per square inch gauge (psig) to about 150 psig, such as from about 50 psig to about 100 psig, such as from about 50 psig to about 75 psig such that the C4 fractions may be condensed with cooling tower water. The fractionation column(s) reboiler may be operated such that isobutylene at greater than about 99% purity may be taken overhead at about 19° F.-21° F. (from about −7.2° C. to about −6.1° C.) and 2-butene may be taken as a bottoms stream leaving the fractionation column column(s) at greater than about 31° F. (greater than about 1.1° C.). If isobutane and/or n-butane is present, isobutane at greater than 90% purity may be taken as a top side draw at about 10° F. (about −12° C.) to about 12° F. (about −11° C.) and n-butane may be taken as a bottom side draw at about 29° F.-31° F. (from about −1.7° C. to about −0.6° C.).

The n-butane fraction, if any, may exit the separation unit 125 and may be collected in storage tanks or flowed into a pipeline where the n-butane fraction may be utilized in other chemical operations or burned as fuel. The isobutane fraction, if any, may exit the separation unit 125 and may serve as a solvent makeup for a PIB unit feed (also referred to as a PIB reactor feed).

The 2-butene fraction may exit the separation unit 125 through line L6 and may be flowed to the skeletal isomerization reactor 115. Skeletal isomerization is described herein. The skeletal isomerization converts the 2-butene to isobutylene. Recycling of the 2-butene back to the skeletal isomerization reactor 115 further increases conversion of the C4-containing feed into isobutylene.

An isobutylene fraction (isobutylene feed), which may be high purity isobutylene, exiting the separation unit 125 through line L7 may be flowed to a polymerization unit 130 through a pump (not shown). The polymerization unit 130 includes a polymerization reactor (also referred to herein as a polyisobutylene (PIB) reactor). The polymerization reactor may include any suitable reactor such as a tubular loop reactor. The polymerization reactor of the polymerization unit 130 is configured to form PIB, such as HR-PIB. The isobutylene may be optionally diluted to 85-95% with any suitable non-polar hydrocarbon diluent such as isobutane, hexane, or combinations thereof. An optional reactor circulation loop (not shown) may be coupled to the polymerization reactor of the polymerization unit 130. The mixture comprising isobutylene and polymerization catalyst present in the polymerization reactor of the polymerization unit 130 may be recirculated in the optional reactor circulation loop, for example, to provide high velocity, with use of an in-line circulation pump (not shown). Additionally, or alternatively, the isobutylene feed may be fed directly to the polymerization reactor of the polymerization unit 130.

Polymerization catalyst, held in polymerization catalyst unit 140, may be fed into the polymerization reactor of the polymerization unit 130 through line L12. The polymerization catalyst may be injected as a slurry of solid polymerization catalyst in a suitable diluent/solvent such as an alkane, such as hexane, octane, or combinations thereof. The polymerization catalyst may include any suitable polymerization catalyst to convert isobutylene to PIB, such as HR-PIB, such as those polymerization catalysts described herein. For example, the polymerization catalyst may include a Lewis acid, such as BF₃, a BF₃ complex, and/or other suitable polymerization catalysts. The polymerization catalyst may be a liquid, a gas, a solid (such as powders), or combinations thereof. The polymerization catalyst may be injected or otherwise introduced into the polymerization reactor of the polymerization unit 130 using an optional catalyst feed pump (not shown). Additionally, or alternatively, the polymerization catalyst may be fed directly to the polymerization reactor of the polymerization unit 130.

In the polymerization reactor of the polymerization unit 130, a mixture is formed that includes polymerization catalyst and isobutylene, and optionally diluent. Upon reaction of the mixture, a polymerization product effluent (also referred to herein as a crude reaction mixture) is formed that includes a polymer composition. The polymer composition may be or include PIB, such as HR-PIB.

The polymerization reaction in the polymerization reactor of the polymerization unit 130 is performed under conditions effective to form a polymerization product effluent comprising the polymer composition. Suitable polymerization catalysts and polymerization conditions may include those described in U.S. Pat. Nos. 10,640,590, 11,124,585, 11,214,637, and 11,174,206, each of which is incorporated in its entirety to the extent consistent with embodiments of the present disclosure.

As an illustrative, but non-limiting, example, isobutylene (or an isobutylene feed) flows into the polymerization reactor of the polymerization unit 130 (for example, a PIB reactor or an HR-PIB reactor) in which the mixture comprising isobutylene and polymerization catalyst may be recirculated in a tubular loop reactor at high linear velocities utilizing an optional in-line circulation pump. The tubular loop reactor may be of a tube-in-shell design. The polymerization reaction occurs in the tubes, and the cooling medium may be provided by an external chiller unit which flows on the shell side to control the polymerization reaction temperature. A polymerization catalyst may be injected through line L12 flowing from polymerization catalyst unit 140 with the assistance of an optional catalyst feed pump (not shown). The polymerization reaction is exothermic and takes place in the liquid phase in the polymerization reactor tubes at a pressure that may be greater than the autogenous pressure at the given reactor temperature, which may be in a range from about 100 psig to about 150 psig (from about 700 kPa to about 1,000 kPa). The reaction temperature of the polymerization may be controlled by coolant flow on a shell side of the polymerization reactor supplied by external cooling unit (not shown). The residence time in the polymerization reactor of the polymerization unit 130 may be determined by the isobutylene feed rate, which is independent of the circulation rate provided by an in-line circulation pump. The polymerization product effluent exiting the polymerization reactor of the polymerization unit 130 includes PIB (for example, HR-PIB). The polymerization product effluent may be a crude reaction mixture comprising the polymer composition (for example, PIB, such as HR-PIB) and one or more optional components. The one or more optional components of the polymerization product effluent may include isobutylene oligomer coproducts, unreacted isobutylene, hydrocarbon diluent (e.g., isobutane, hexane, or combinations thereof), polymerization catalyst residues, or combinations thereof. The one or more optional components of the polymerization product effluent may be recycled to various units in the system 100.

The polymerization catalyst may be injected (for example, through line L12) into the incoming isobutylene feed flowing through line L7 and into the optional reactor circulation loop at a point where the physical distance between the injection point in the feed line and the point at which the feed enters the polymerization reactor of the polymerization unit 130 may be at a minimum. The injection point for the polymerization catalyst may be on a suction side of the feed pump to provide mixing. The polymerization catalyst may be introduced at a concentration sufficient to catalyze an isobutylene polymerization reaction.

The polymerization in the polymerization reactor of the polymerization unit 130 may be performed according to the following non-limiting procedure. A polymerization catalyst feed and an isobutylene feed may be flowed into the polymerization reactor of the polymerization unit 130 to form a mixture comprising isobutylene and polymerization catalyst. The mixture comprising isobutylene and polymerization catalyst may be maintained at a temperature that may be in a range from about −35° C. to about 100° C., such as from about 0° C. to about 80° C. or from about −10° C. to about 40° C. The reaction may be carried out in the liquid phase at a pressures of at least about autogenous pressures, such as in a range from about 15 psig to about 150 psig (from about 0.10 MPa to about 1.0 MPa), such as from about 100 psig to about 150 psig (from about 0.7 MPa to about 1.0 MPa). After a suitable period, a polymer composition is obtained. For example, the polymerization may be performed for a polymerization period of about 30 minutes or less, such as about 10 minutes or less, such as about 4 minutes or less, such as about 2 minutes or less. The polymerization period refers to a residence time of the mixture comprising the isobutylene and polymerization catalyst in the polymerization reactor.

The polymerization reactor of the polymerization unit 130 may include any suitable reactor. Suitable polymerization reactors useful for the polymerization may include a batch reactor, a continuous flow reactor, a tank reactor, a tubular reactor, a tubular loop reactor, a continuous stirred tank reactor (CSTR), a plug flow reactor, a fluidized bed reactor, an immobilized bed reactor, a fixed bed reactor, or combinations thereof, such as such as a continuous flow reactor, a CSTR, a tubular reactor, a tubular loop reactor, or combinations thereof.

The polymerization reactor may include more than one reactor which may be operated in series or parallel. The polymerization reactor(s) may or may not have internal cooling or heating, and the feeds to the polymerization reactor may or may not be refrigerated.

Times and temperatures may be controlled such that no significant olefin isomerization occurs during polymerization and/or such that conversion and molecular weights are in desirable ranges. Reaction temperatures and pressures, and polymer precursor concentrations, may be selected to control for the number average molecular weight (Mn) of the polymer composition. For example, higher temperatures typically provide polymer compositions with lower Mn. In contrast, lower temperatures (e.g., near sub-ambient levels) may facilitate high selectivity towards the formation of HR-PIB. In addition, and as described herein, the polymerization may occur with very short residence times, for example, down to about 4 minutes or less. Typically, shorter residence times may facilitate a high conversion % of isobutylene to HR-PIB and/or may minimize oligomer coproduct formation.

Temperature control in the polymerization reactor of the polymerization unit 130 may be achieved by offsetting the heat of polymerization with reactor cooling by using reactor jackets or cooling coils to cool the contents of the polymerization reactor, auto refrigeration, pre-chilled feeds, vaporization of liquid medium (diluent, polymer precursors, or solvent), or combinations thereof. In the case of CSTR with ebullient cooling, the boiling mixture may be cooled with a chilled overhead condenser. For non-ebullient cooled CSTR, any suitable type of heat exchanger may be used to chill the reactor jacket using any suitable cooling media.

In some embodiments, which may be combined with other embodiments, a fast reactor is used. A fast reactor is one in which the reactor is the heat exchanger with the reaction taking place in the tubes with cooling on the shell. Any suitable type of cooling media may be used depending mainly on operating temperature range. Adiabatic reactors with pre-chilled feeds may also be used. In some embodiments, which may be combined with other embodiments, the reactor(s) for the polymerization may be operated in as much of an isothermal mode as possible. Non-isothermal reactor operation may result in broader molecular weight distributions. In series operation with two or more polymerization reactors, a second reactor temperature may be higher than a first reactor temperature. In parallel reactor operation, the temperatures of the two reactors may be independent.

For CSTR operations, a concentration of the polymerization catalyst in the mixture comprising isobutylene and polymerization catalyst may be from about 1,000 ppm to about 2,000 ppm based on a total weight of the polymerization catalyst feed (fed through line L12), wherein a Lewis acid concentration (e.g., BF₃) in the polymerization catalyst feed may be in a range from about 250 ppm to about 1,000 ppm, such as from about 250 ppm to about 500 ppm or from about 500 ppm to about 1,000 ppm based on the total weight of the polymerization catalyst feed. Residence times of the mixture comprising the isobutylene and polymerization catalyst may be less than about 600 minutes, such as less than about 120 minutes, such as less than about 60 minutes, or in a range from about 5 minutes to about 600 minutes, such as from about 15 minutes to about 120 minutes, such as from about 30 minutes to about 60 minutes, and may be controlled by polymerization catalyst concentration. Higher polymerization catalyst concentrations may increase the reaction rate. The polymerization reaction may be highly exothermic and a limiting factor to reaction rate may include the ability to remove the heat of reaction.

In CSTR operations, the mixture comprising the polymerization catalyst and isobutylene may be flowing upward in the polymerization reactor, through at least a first portion of the polymerization reactor and a second portion of the polymerization reactor. The first portion of the polymerization reactor may be relatively narrow to provide higher velocity and higher polymerization catalyst mixing. The second portion of the polymerization reactor may be wider to provide lower velocity and less polymerization catalyst mixing. The polymerization product effluent may exit near the top of the polymerization reactor with some polymerization catalyst being carried out with the exiting polymerization product effluent. The polymerization catalyst exiting the polymerization reactor may be made up with polymerization catalyst from a polymerization catalyst injection such that a constant polymerization catalyst amount is maintained in the polymerization reactor. The reaction temperature may be maintained by vaporization of a portion of the isobutylene feed controlled by the reactor pressure. For example, a higher polymerization reactor pressure may give higher reaction temperature according to the vapor pressure curve of the system butylenes. Mn of the polymer composition may be controlled by reaction temperature with a higher reaction temperature giving lower Mn. Reaction temperatures in a range from about −5° C. to about 5° C. may provide polymer compositions having an Mn in a range from about 2,100 Daltons (Da) to about 2,500 Da, such as 2,300 Da. Reaction temperatures in a range from about 18° C. to about 22° C. may provide polymers having an Mn in a range from about 900 Da to about 1,100 Da, such as about 1,000 Da.

For fast reactor modes, the polymerization reactor of the polymerization unit 130 may include tubular loop reactor. Here, the tubular loop reactor may include a tube-in-shell heat exchanger with the polymerization reaction taking place in the tubes and cooling provided through the shell side of the heat exchanger with the heat of the polymerization reaction taken out by an external chiller unit. A reactor design for the polymerization reactor of the polymerization unit 130 may include a two-pass heat exchanger. The reaction may be carried out in the liquid phase at pressures of at least about autogenous pressures, typically greater than about 0 psig (0 kPa), such as in a range from about 35 psig to about 300 psig (from about 250 kPa to about 2100 kPa), such as from about 100 psig to about 150 psig (from about 700 kPa to about 1000 kPa).

A concentration of the polymerization catalyst in the polymerization reaction mixture (e.g., the mixture comprising isobutylene and polymerization catalyst) may be from about 800 ppm to about 10,000 ppm based on a total weight of the polymerization reaction mixture, and wherein a BF₃ concentration in the polymerization reaction mixture may be from about 500 ppm to about 7,250 ppm based on the total weight of the polymerization reaction mixture. Additionally, or alternatively, the concentration of the polymerization catalyst in the polymerization reaction mixture may be from about 2,000 ppm to about 4,000 ppm based on a total weight of the polymerization reaction mixture, and wherein the BF₃ concentration in the polymerization reaction mixture may be from about 1,250 ppm to about 2,900 ppm based on the total weight of the polymerization reaction mixture. Additionally, or alternatively, the concentration of the polymerization catalyst in the polymerization reaction mixture may be greater than about 800 ppm based on a total weight of the polymerization reaction mixture, and wherein the BF₃ concentration in the polymerization reaction mixture may be greater than about 500 ppm based on a total weight of the polymerization reaction mixture.

A tubular loop reactor useful for the polymerization reactor of the polymerization unit 130 may include a multi-pass tube-in-shell heat exchanger configuration Such a configuration supports the maintenance suitable reaction conditions. Other tubular loop reactors are contemplated and may include a single pass tube-in-shell heat exchanger configuration. The tubular loop reactor may be equipped with in-line circulation pumps that provide high flow rates, ensuring turbulent mixing and uniform dispersion of the catalyst and solid substrate. Temperature control may be achieved through the circulation of cooling media, which regulates the exothermic polymerization reaction and sorption processes. This thermal management may serve to enhance the stability and efficiency of the polymerization, preventing catalyst degradation and side reactions.

For a tubular loop reactor, a circulation loop may be provided to deliver high velocity in the tubes at a Reynolds number of the circulating liquid in the tubes that may be about 2,000 or more. Reynolds numbers of about 2,000 or more allow for turbulent flow in the tubes which increases the heat exchange and the ability to remove the heat of reaction in very short periods of time. The ability to quickly remove the heat of reaction allows for operation at very short residence times. A residence time of the polymerization reaction mixture (the mixture comprising the isobutylene and polymerization catalyst) in the tubular loop reactor may be about 60 minutes or less, such as about 30 minutes or less, such as about 10 minutes or less, such as about 4 minutes or less, such as about 3 minutes or less, such as about 2 minutes or less, such as about 1 minute or less. Additionally or alternatively, a residence time of the polymerization reaction mixture in the tubular loop reactor may be in a range from about 1 second to about 60 minutes, such as from about 5 seconds to about 30 minutes, such as from about 10 seconds to about 10 minutes, such as from about 30 seconds to about 4 minutes.

The tubular loop reactor may be designed for high-velocity movement of reaction mixtures (for example, the mixture comprising isobutylene and polymerization catalyst). The high-velocity movement may provide one or more advantages, for example, enhanced heat transfer/removal and efficient catalyst dispersion. The high velocity (e.g., a velocity in a range from about 5 m/s to about 30 m/s) of the mixture comprising isobutylene and polymerization catalyst may help ensure that heat generated during the exothermic polymerization reaction may be quickly dissipated, preventing localized hot spots and enabling the reaction to proceed under a controlled temperature, e.g., a temperature in a range from about −10° C. to about 40° C. The tubular design may also help ensure uniform polymerization catalyst distribution, enhancing the contact between isobutylene and the polymerization catalyst, which may be useful for maintaining high polymerization efficiency and product uniformity. The heat transfer/removal and catalyst dispersion may help facilitate control (or precise control) of molecular weight distribution and/or may help facilitate high selectivity for terminal double bonds in the PIB product.

The polymerization reaction may occur with very short residence times, for example, less than about 4 minutes, to minimize coproduct formation and ensure a high conversion rate of isobutylene to HR-PIB. Operating pressures may be kept within a range from about 0.1 MPa to about 1 MPa, depending on the specific polymerization requirements and feedstock quality. By maintaining a lower temperature (e.g., near sub-ambient levels), high selectivity towards the formation of HR-PIB may be achieved.

Tubular loop reactors useful with embodiments described herein may employ an optional reactor circulation loop, such as those described in U.S. Patent Application Publication No. 2024/0425624, which is incorporated in its entirety to the extent consistent with embodiments of the present disclosure.

The optional reactor circulation loop (where the mixture comprising isobutylene and polymerization catalyst is present) may be independent of a feed flow of an isobutylene feed to the polymerization reactor (e.g., the isobutylene feed in line L7) such that a velocity of the mixture comprising isobutylene and polymerization catalyst in the tubular loop reactor may be in a range from about 3 ft/sec (about 0.9 m/sec) or more, such as in a range from about 6 ft/sec to about 10 ft/sec (from about 1.8 m/see to about 3.05 m/s). Such velocities facilitate turbulent flow.

The optional reactor circulation loop and isobutylene feed flow are typically controlled by different pumps. The circulation loop where the mixture comprising isobutylene and polymerization catalyst is present may be controlled by an in-line circulation pump and the feed flow may be controlled by a separate pump that controls the flow of the isobutylene feed into line L7 and into the polymerization reactor of the polymerization unit 130.

When the optional reactor circulation loop is utilized, a ratio of the circulation flow of the mixture comprising isobutylene and polymerization catalyst in the optional reactor circulation loop to the flow of the isobutylene feed in line L7 may be in a range from about 10:1 or more, such as from about 10:1 to about 50:1. Such ratios facilitate turbulent flow.

In at least one embodiment, which may be combined with other embodiments, the polymerization occurs in a high-speed reactor, such as a fast reactor. As an example, the polymerization operation may be performed by the following procedure. High purity isobutylene may be fed to a tubular loop reactor and mixed in situ with a polymerization catalyst described herein such that the polymerization catalyst concentration may be in a range of from about 1,000 ppm to about 10,000 ppm, such as from about 2,000 ppm to about 10,000 ppm based on the total weight of the polymerization reaction mixture (the mixture comprising the isobutylene and polymerization catalyst). The residence time in the polymerization reactor of the polymerization unit 130 may be less than about 4 minutes. The polymerization product effluent comprising HR-PIB may then exit the polymerization reactor of the polymerization unit 130.

The polymerization reaction in the polymerization reactor of the polymerization unit 130 converts at least a portion of isobutylene in an isobutylene feed that enters the polymerization reactor of the polymerization unit 130 through line L7 to a polymerization product effluent comprising PIB (for example, HR-PIB). The percent conversion to PIB (or HR-PIB) may be about 50% or more, such as about 60% or more, such as about 70% or more, or in a range from about 75% to about 100%, such as from about 85% to about 95% based on an amount of isobutylene entering the polymerization reactor of the polymerization unit 130. Additionally, or alternatively, the percent conversion to PIB (or HR-PIB) may be about 90% or more, such as about 91% or more, such as about 92% or more, such as about 93% or more, such as about 94% or more, such as about 95% or more, such as about 96% or more, such as about 97% or more, such as about 98% or more, such as about 99% or more, such as about 100% based on an amount of isobutylene entering the polymerization reactor of the polymerization unit 130.

The polymer composition (for example, PIB such as HR-PIB) produced by the polymerization may have any suitable Mn. For example, the PIB may have an Mn of about 180 Da or more, such as about 240 Da or more, such as about 300 Da or more, such as about 360 Da or more, such as about 420 Da or more, such as about 480 Da or more, such as in a range from about 180 Da to about 480 Da. Additionally, or alternatively, the PIB may have an Mn in a range from about 320 Da to about 10,000 Da, such as from about 320 Da to about 5,000 Da, such as from about 350 Da to about 5,000 Da, such as from about 700 Da to about 2,250 Da, or from about 350 Da to about 2,250, such as from about 700 Da to about 950 Da, or from about 1300 Da to about 2,250 Da.

The PIB may have any suitable polydispersity index (PDI). PDI is the ratio of Mw to Mn. The PDI (Mw/Mn) of the PIB may be about 5 or less, such as about 2.5 or less, about 2 or less, about 1.5 or less, or about 1.3 or less.

The polymer composition (for example, PIB) may include a first portion comprising polymer chains having alpha vinylidene groups (alpha vinylidene olefin isomers), a second portion comprising polymer chains having beta vinylidene groups (beta vinylidene olefin isomers), and a third portion comprising polymer chains having internal vinylidene groups (internal vinylidene olefin isomers).

As described herein, the PIB may be an HR-PIB. HR-PIB is a PIB where:
(a) the first portion comprising polymer chains having alpha vinylidene groups may be 75 wt % or more, such as about 80 wt % or more, such as about 82 wt % or more, such as about 85 wt % or more, such as about 87 wt % or more, such as about 90 wt % or more, such as about 92 wt % or more, such as about 94 wt % or more, such as about 95 wt % or more based on a total wt % of the PIB, the total wt % of the PIB equal to 100 wt %; and
(b) a total of the second portion comprising polymer chains having beta vinylidene groups plus the third portion comprising polymer chains having internal vinylidene group may be 25 wt % or less, such as about 20 wt % or less, such as about 18 wt % or less, such as about 15 wt % or less, such as about 13 wt % or less, such as about 10 wt % or less, such as about 8 wt % or less, such as about 6 wt % or less, such as about 5 wt % or less based on the total wt % of the PIB.

Alternatively, the polymer composition may be a mid-range vinylidene PIB. Mid-range vinylidene PIBs have an alpha vinylidene content less than 75 wt %, such as about 65% or less based on the total wt % of the PIB. In these and other embodiments, the mid-range vinylidene PIB may include: a first portion comprising polymer chains having alpha vinylidene groups, a second portion comprising polymer chains having beta vinylidene groups, and a third portion comprising polymer chains having internal vinylidene groups, wherein: the first portion may be less than 75 wt %, such as from greater than 10 wt % to less than 75 wt %, such as from 40 wt % to less than 75 wt % such as from about 50 wt % to about 65 wt % based on the total wt % of the PIB; and the second portion plus the third portion may be from greater than about 25 wt % to about 60 wt % or less, such as from about 35 wt % to about 50 wt % based on the total wt % of the PIB.

The polymerization catalyst facilitates or promotes polymerization of the isobutylene present in the mixture. The polymerization catalyst may include any suitable Lewis acid catalyst, Lewis acid catalyst complex, or combinations thereof. A “catalyst complex” refers to a complex of a catalyst and a complexing agent. For example, a Lewis acid catalyst complex includes a Lewis acid catalyst and a complexing agent.

The polymerization catalyst may be a liquid, a gas, a solid (such as powders), or combinations thereof. Suitable Lewis acid catalysts useful as a polymerization catalyst may include a fluorine-containing material, a chlorine-containing material, or combinations thereof. Suitable fluorine-containing materials may include BF₃, tris(pentaflurophenyl) borane (C₆F₅)₃B, or a combination thereof. Suitable chlorine-containing materials may include a metal chloride (for example, AlCl₃, ZnCl₂, SnCl₄, TiCl₄), a metal alkyl chloride (for example, ethylaluminum dichloride (EtAlCl₂)), or combinations thereof.

Suitable Lewis acid catalyst complexes include a Lewis acid and a complexing agent. The Lewis acid catalyst may include one or more of those described herein. The complexing agent may include an oxygen-containing compound (also referred to as an oxygenate).

Oxygen-containing compounds may include an alcohol, an ether, a ketone, an aldehyde, a carboxylic acid, or combinations thereof. The complexing agent may include a C1 to C10 unsubstituted alcohol, a C1 to C10 substituted alcohol, a C2 to C20 unsubstituted ether, or a C2 to C20 substituted ether. The complexing agent may include more than one oxygen containing group per molecule, for example, glycols (substituted or unsubstituted) and polyols (substituted or unsubstituted), for example wherein each hydroxyl is in a primary position, or for example, a C1 to C10 glycol (substituted or unsubstituted) such as ethylene glycol, 1,4-butanediol, trimethylolethane (2-(hydroxymethyl)-2-methylpropane-1,3-diol; C₅H₁₂O₃), trimethylolpropane (2-(hydroxymethyl)-2-ethylpropane-1,3-diol; C₆H₁₄O₃), pentaerythritol (2,2-bis(hydroxymethyl)propane-1,3-diol; C₅H₁₂O₄), or tris(hydroxymethyl)aminomethane (C₄H₁₁NO₃).

Oxygen-containing compounds may include methanol (MeOH), ethanol, isopropanol, n-propanol, neopentyl alcohol, dimethyl ether, diethyl ether, diisopropyl ether, diisobutyl ether, di-tert-butyl ether, methyl tert-butyl ether, ethylene glycol, neopentyl alcohol, 2,2-dimethylbutanol, 2,2-dimethylpentanol, 2,2-dimethylhexanol, benzyl alcohol, a ring-substituted benzyl alcohol, or combinations thereof. The complexing agent may include an alcohol that lacks (or is free of) beta-hydrogen atoms. Alcohols with a beta hydrogen atom may undergo an undesired dehydration/beta-elimination reactions. Beta-elimination reactions may destabilize the catalyst and reduce its efficiency in the polymerization.

Accordingly, use of oxygen-containing compounds that are free of beta-hydrogen atoms prevent, or at least mitigate, beta-elimination reactions during polymerization of the isobutylene. Oxygen-containing compounds that lack beta hydrogen atoms may include methanol, a 2,2-dimethyl alcohol (for example, neopentyl alcohol, 2,2-dimethylbutanol, 2,2-dimethylpentanol, and 2,2-dimethylhexanol), benzyl alcohol, a ring-substituted benzyl alcohol, or combinations thereof.

A catalyst complex may be formed by adding a Lewis acid catalyst to an oxygen-containing compound. For example, a catalyst complex comprising BF₃/complexing agent (such as BF₃·MeOH) may be formed by passing BF₃ gas through a pure anhydrous oxygen-containing compound at a rate that allows the BF₃ to be efficiently contacted by the oxygen-containing compound. An illustrative, but non-limiting, example of a Lewis acid catalyst complex may include BF₃·MeOH.

A catalyst complex comprising a Lewis acid catalyst/complexing agent (for example, BF₃/complexing agent) may include any suitable molar ratio of complexing agent to Lewis acid catalyst. For example, a catalyst complex may include a molar ratio of complexing agent to Lewis acid catalyst that may be in a range from about 0.1 to about 10, such as from about 0.2 to about 5, such as from about 0.2 and 2, such as from about 0.5 to about 2, such as from about 1.0 to about 1.9, such as from about 1.0 to about 1.3, for example, about 1.0, or from about 1.3 to about 1.5, such as about 1.4.

The polymerization catalyst may be a solid polymerization catalyst. The solid polymerization catalyst may include a polymerization catalyst sorbed on a solid substrate. “Sorbed” may refer to (1) adsorbed (for example, surface attached) polymerization catalyst, (2) absorbed polymerization catalyst, (3) or combinations thereof. The solid substrate serves as a support material for the polymerization catalyst. The solid substrate may include any suitable solid substrate capable of supporting a polymerization catalyst described herein, for example, a Lewis acid catalyst and/or Lewis acid complex.

The solid substrate may form a stable adduct with the polymerization catalyst. The solid substrate may be a porous solid substrate, Suitable solid substrates may include inorganic oxides, metal oxides doped with rare earth metals, rare earth metals themselves, or combinations thereof. The solid substrates may include an inorganic oxide in a finely divided form, such as a powder. Suitable solid substrates may include metal oxides of Group IIIA, Group IVA, and/or Group IVB of the periodic table of the elements, such as alumina, silica, titania, or mixtures thereof. Combinations of solid substrates may be used, for example, silica-alumina and silica-titania. Illustrative, but non-limiting, solid substrates may include, or be selected from, Al₂O₃, ZrO₂, TiO₂, SnO₂, CeO₂, SiO₂, SiO₂/Al₂O₃, or combinations thereof, such as SiO₂, Al₂O₃, SiO₂/Al₂O₃ (silica-alumina), or combinations thereof.

The solid substrate may have one or more of the following properties:
(a) The solid substrate may include an Al₂O₃ content that is greater than 0 wt %, such as greater than about 1 wt %, such as greater than about 3 wt %, such as greater than about 5 wt %, such as greater than about 10 wt %, such as greater than about 15 wt %, such as greater than about 20 wt %, such as greater than about 25 wt %, such as greater than about 30 wt %, such as greater than about 35 wt %, such as greater than about 40 wt %, such as greater than about 45 wt %, such as greater than about 50 wt % based on the total wt % of the solid substrate. The total wt % of the solid substrate is equal to 100 wt %. Additionally, or alternatively, the solid substrate may include an Al₂O₃ content that is less than 100 wt % SiO₂, such as less than about 99 wt %, such as less than about 97 wt %, such as less than about 95 wt %, such as less than about 90 wt %, such as less than about 85 wt %, such as less than about 80 wt %, such as less than about 75 wt %, such as less than about 70 wt %, such as less than about 65 wt %, such as less than about 60 wt %, such as less than about 55 wt %, such as less than about 50 wt % based on the total wt % of the solid substrate. Additionally, or alternatively, the solid substrate may include an Al₂O₃ content that is in a range from about 25 wt % to about 75 wt %, such as from about 25 wt % to about 50 wt % based on a total wt % of the solid substrate. Additionally, or alternatively, the solid substrate may include an Al₂O₃ content that is within those aforementioned weight percents.
(b) The solid substrate may include an SiO₂ content that is greater than 0 wt %, such as greater than about 1 wt %, such as greater than about 3 wt %, such as greater than about 5 wt %, such as greater than about 10 wt %, such as greater than about 15 wt %, greater than about 20 wt %, such as greater than about 25 wt %, such as greater than about 30 wt %, greater than about 35 wt %, such as greater than about 40 wt %, such as greater than about 45 wt %, such as greater than about 50 wt % based on the total wt % of the solid substrate. Additionally, or alternatively, the solid substrate may include an SiO₂ content that is less than 100 wt %, such as less than about 99 wt % SiO₂, such as less than about 97 wt %, such as less than about 95 wt %, such as less than about 90 wt %, such as less than about 85 wt %, such as less than about 80 wt %, such as less than about 75 wt %, such as less than about 70 wt %, such as less than about 65 wt %, less than about 60 wt %, such as less than about 55 wt %, such as less than about 50 wt % based on the total weight of the solid substrate. Additionally, or alternatively, the solid substrate may include an SiO₂ content that is within those aforementioned weight percents.
(c) The solid substrate may be characterized as having a surface area that is greater than about 10 m²/g, about 750 m²/g or less, or a combination thereof, such as in a range from about 10 m²/g to about 700 m²/g, such as from about 50 m²/g to about 500 m²/g, such as from about 100 m²/g to about 400 m²/g. Additionally, or alternatively, the solid substrate may be characterized as having surface area that is greater than about 150 m²/g (with a maximum that may be about 750 m²/g).
(d) The solid substrate may be characterized as having a pore volume that is greater than about 0.1 cc/g, such as in a range from about 0.1 cc/g to about 4.0 cc/g, such as from about 0.5 cc/g to about 3.5 cc/g, such as from about 0.8 cc/g and about 3.0 cc/g.
(e) The solid substrate may be characterized as having a monodispersed particle size or a distribution of particle sizes with an average particle size that is greater than about 5 μm, such as in a range from about 5 μm to about 500 μm, such as from about 5 μm to about 200 μm, such as from about 10 μm to about 100 μm.
(f) The solid substrate may be characterized as having an average pore size (diameter) that is greater than about 1 nm, such as in a range from about 1 nm to about 100 nm, such as from about 5 nm to about 75 nm, such as from about 7.5 nm and about 48 nm, or from about 20 nm to about 150 nm.
(g) The solid substrate may be characterized as having a pore volume that is greater than about 0.3 cc/g, such as in a range from about 0.3 cc/g to about 2.0 cc/g, such as from about 0.5 cc/g to about 1.75 cc/g, such as from about 0.75 cc/g to about 1.5 cc/g.
(h) The solid substrate may be characterized as having an amount of Fe₂O₃ that is less than about 5 wt %, such as less than about 1 wt %, such as less than about 0.5 wt %, such as less than about 0.2 wt %, such as 0 wt % based on the total wt % of the solid substrate.
(i) The solid substrate may be characterized as having an amount of Na₂O that is less than about 5 wt % such as less than about 1 wt %, such as less than about 0.5 wt %, such as less than about 0.2 wt %, such as less than about 0.02 wt %, such as 0 wt % based on the total wt % of the solid substrate.

The solid substrate may include, for example, a high surface area, amorphous silica (for example, a surface area in a range from about 250 m²/g to about 350 m²/g, such as about 300 m²/g, and a pore volume in a range from about 1.3 cc/g to about 2.0 cc/g, such as about 1.65 cc/g).

Illustrative, but non-limiting, examples of solid substrates may include one or more of the following: ALS 75 SiO₂/Al₂O₃ (silica-alumina) commercially available from Pacific Industrial Development Corporation, Ann Arbor, Michigan; gamma-alumina spheres (γ-Al₂O₃) commercially available from BASF; ALS 50 SiO₂/Al₂O₃ (silica-alumina) commercially available from Pacific Industrial Development Corporation; or combinations thereof. ALS 75 includes about 75 wt % SiO₂ and about 25 wt % Al₂O₃. ALS 50 includes about 50 wt % SiO₂ and about 50 wt % Al₂O₃.

An illustrative, but non-limiting, polymerization catalyst may include a solid BF₃ catalyst complex. A solid BF₃ catalyst complex includes a solid substrate, such as one or more of the solid substrates described herein. The solid BF₃ catalyst further includes BF₃ and a complexing agent (for example, an oxygen-containing compound) described herein. For example, the polymerization catalyst may include a solid BF₃·MeOH catalyst complex (a BF₃·MeOH catalyst complex supported on a solid substrate).

The solid substrate comprising sorbed BF₃ catalyst complex (for example, BF₃-MeOH catalyst complex) may include any suitable amount of concentration of BF₃, for example, about 1 wt % or more, such as about 5 wt % or more, such as about 10 wt % or more, such as about 15 wt % or more, such as about 20 wt % or more, such as about 25 wt % or more, such as about 30 wt % or more, such as about 35 wt % or more, such as about 40 wt % or more, such as about 45 wt % or more, such as about 50 wt % or more, such as about 55 wt % or more, such as about 60 wt % or more, such as about 65 wt % or more, such as about 70 wt % or more, such as about 75 wt % or more, such as about 80 wt % or more, such as about 85 wt % or more, such as about 90 wt % or more, such as about 95 wt % or more based on a total wt % of the solid BF₃ catalyst complex (calculated as wt % of BF₃). The total wt % of the solid BF₃ catalyst complex is equal to 100 wt %. Any of the foregoing numbers may be used singly to describe an open-ended range or in combination to describe a close-ended range.

The amount of BF₃ is calculated based on the BF₃ and not including the oxygenated compound of the complex because the oxygenated compound may not just be MeOH but may include additional or alternative oxygenated compounds.

Higher BF₃ loadings (for example about 30 wt % or more) may help ensure sustained catalytic activity throughout the polymerization process. The use of a solid catalyst system (solid substrate comprising sorbed BF₃ catalyst complex) may not only prevent degradation of the product but may also eliminate the need for water washing and the handling of toxic BF₃ gas on-site, significantly improving operational safety and reducing environmental impact.

The BF₃ catalyst complex may include any suitable molar ratio of complexing agent to BF₃. For example, a BF₃ catalyst complex may include a molar ratio of complexing agent to BF₃ that may be in a range from about 0.1 to about 10, such as from about 0.2 to about 5, such as from about 0.2 and 2, such as from about 0.5 to about 2, such as from about 1.0 to about 1.9, such as from about 1.0 to about 1.3, for example, about 1.0, or from about 1.3 to about 1.5, such as about 1.4 (complexing agent:BF₃).

As described herein, the polymerization product effluent may be a crude reaction mixture comprising the polymer composition (for example, PIB, such as HR-PIB) and one or more optional components. The one or more optional components of the polymerization product effluent may include isobutylene oligomer coproducts, unreacted isobutylene, hydrocarbon diluent (e.g., isobutane, hexane, or combinations thereof), polymerization catalyst residues, or combinations thereof. The one or more optional components of the polymerization product effluent may be recycled to various units in the system 100.

The polymerization unit 130 may further include any suitable apparatus to purify the polymerization product effluent and separate the one or more optional components from the polymerization product effluent. The polymerization product effluent may be purified by separation, atmospheric stripping, vacuum stripping, or a combination thereof to remove coproducts, unreactive compounds, catalyst residues, and unreacted polymer precursors. Unreacted polymer precursors may be recycled. For example, such purification may be accomplished in a system by passing the crude polymer composition through a solid-liquid separation device and then through a pressure distillation column to remove the unreacted polymer precursors and other non-reacted residues. The distillation columns may be atmospheric and/or vacuum distillation columns. Such embodiments are described below.

For example, polymerization catalyst residues may be separated from the polymerization product effluent at polymerization unit 130 using any suitable solid-liquid separation technique to separate the polymerization catalyst residues (solids) from the polymerization product effluent. Suitable solid-liquid separation apparatus, may include apparatus for performing filtration, vacuum filtration, centrifugation, decanting, decanting centrifugation, or combinations thereof. The polymerization catalyst residues (solids) may be recovered.

As another example, isobutane, unreacted isobutylene, or combinations thereof may be removed from the polymerization product effluent at polymerization unit 130 by use of, for example, a debutanizer column, debutanizer fractionator, a distillation column, a fractional distillation column, or combinations thereof. Separation of isobutane, unreacted isobutylene, or combinations thereof may be accomplished utilizing any suitable conditions effective to remove the isobutane, unreacted isobutylene, or combinations thereof from the polymerization product effluent. For example, the polymerization product effluent may be passed through a distillation column in polymerization unit 130 operating at weight hourly space velocity (WHSV) that may be from about 1 h⁻¹ to about 60 h⁻¹ (or more), at a column temperature that may be from about 25° C. to about 100° C., and a column pressure that may be from about 25 psig to about 100 psig (from about 172 kPa to about 689 kPa).

Isobutane, unreacted isobutylene, or combinations thereof (if any) may exit the polymerization unit 130 where the isobutane, unreacted isobutylene, or combinations thereof may then be combined with the isobutylene feed in line L7. This combined stream may then be introduced into the polymerization reactor of the polymerization unit 130. Additionally, or alternatively, the isobutane, unreacted isobutylene, or combinations thereof may be introduced directly back into the into the polymerization reactor of the polymerization unit 130.

Having removed isobutane, unreacted isobutylene, or combinations thereof (if any) from the polymerization product effluent, an amount of PIB (for example, HR-PIB) in the resultant effluent may be 50 wt % or more, about 70 wt % or more, about 85 wt % or more, about 90 wt % or more, about 91 wt % or more, such as about 92 wt % or more, such as about 93 wt % or more, such as about 94 wt % or more, such as about 95 wt % or more, such as about 96 wt % or more, such as about 97 wt % or more, such as about 98 wt % or more, such as about 99 wt % or more, such as about 100 wt % based on a total wt % of the polymerization product effluent after separating the isobutane, unreacted isobutylene, or combinations thereof.

As another example, the polymerization product effluent may optionally include isobutylene oligomer coproducts, polymerization catalyst solvent (e.g., the component/diluent used to form a polymerization catalyst slurry), or combinations thereof. Isobutylene oligomer coproducts may include C8-C20 isobutylene oligomer coproducts, such as C8-C16 isobutylene oligomer coproducts, such as a C8 isobutylene oligomer coproduct, a C12 isobutylene oligomer coproduct, a C16 isobutylene oligomer coproduct, or combinations thereof. C20 PIB (such as C20 HR-PIB) may be a desirable product. In such instances, C20 PIB may be retained instead.

The isobutylene oligomer coproducts and solvent may be separated from the polymerization product effluent at polymerization unit 130 by use of an oligomer separation unit (not shown), for example, a distillation column, a fractional distillation column, a vacuum distillation column, or combinations thereof. The oligomer separation unit of the polymerization unit 130 may be operated under any suitable conditions effective to remove the one or more optional components, e.g., isobutylene oligomer coproducts, from the polymerization product effluent. Suitable conditions for operating the oligomer separation unit may include passing the polymerization product effluent (after removing isobutane, unreacted isobutylene, or combinations thereof (if any)) through a distillation column operating at a temperature that may be in a range from about 150° C. to about 275° C. and a pressure that may be in a range from about 1 millimeter of mercury (mmHg) to about 100 mmHg (from about 0.1 kPa to about 13 kPa), such as such as from about 10 mmHg to about 50 mmHg (from about 1.3 kPa to about 6.7 kPa).

The isobutylene oligomer coproducts, polymerization catalyst solvent, or combinations thereof (if any) may exit the oligomer separation unit of the polymerization unit 130 (leaving “overhead”) through line L9 where the isobutylene oligomer coproducts, polymerization catalyst solvent, or combinations thereof (if any) may then be fed to a hydrotreatment reactor 135. The polymerization product effluent having removed the optional one or more components (e.g., isobutane, unreacted isobutylene, isobutylene oligomer coproducts, polymerization catalyst, polymerization catalyst solvent, or combinations thereof) may exit the oligomer separation unit of the polymerization unit 130 (leaving as a bottoms “stream”) through line L8. The polymerization product effluent, now purified, may be a highly pure (e.g., >95% pure, such as about 98% or more, such as about 99% or more, such as 100%) PIB composition or HR-PIB composition.

The isobutylene oligomer coproducts flowing through line L9 may enter hydrotreatment reactor 135. At the hydrotreatment reactor 135, the isobutylene oligomer coproducts may be converted to hydrocarbons under hydrotreatment conditions and in the presence of a hydrotreatment catalyst. The hydrotreatment reduces a carbon-carbon double bond present in the isobutylene oligomer coproducts. Some hydroisomerization of the isobutylene oligomer coproducts may occur during hydrotreatment. The hydrocarbons produced from the hydrotreatment may include hydrotreated C8-C20 hydrocarbons, such as hydrotreated C8-C16 hydrocarbons, such as a hydrotreated C8 hydrocarbon, a hydrotreated C12 hydrocarbon, a hydrotreated C16 hydrocarbon, or combinations thereof. The hydrotreatment in the hydrotreatment reactor 135 may be performed with the use of any suitable hydrotreatment catalyst, such as nickel-molybdenum (Ni—Mo), cobalt-molybdenum (Co—Mo), platinum on carbon (Pt/C), palladium on carbon (Pd/C), ruthenium on carbon (Ru/C). Hydrogen (H₂) gas may be fed to the hydrotreatment reactor through line L11 at any suitable pressure, such as a pressure in a range from about 0.5 MPa to about 10 MPa, such as from about 1 MPa to about 8 MPa, depending on, for example, the catalyst. For example, the H₂ gas pressure may be in a range from about 1.5 MPa to about 3 MPa for Pt/C and Pd/C catalysts. The H₂ gas pressure may be in a range from about 2 MPa to about 6 MPa for Ru/C catalysts. The H₂ gas pressure may be in a range from about 4 MPa to about 8 MPa for Ni—Mo and Co—Mo catalysts. The H₂ gas may contact the a mixture comprising the hydrotreatment catalyst and the isobutylene oligomer coproducts, under the hydrotreatment conditions, to form the hydrotreated C8-C20 hydrocarbons. Suitable hydrotreatment conditions may include a temperature in a range from about 200° C. to about 350° C., such as from about 250° C. to about 300° C.; a pressure in a range from about 2 MPa to about 8 MPa, such as from about 4 MPa to about 6 MPa; or combinations thereof. Space velocities may be in a range from about 0.5 h⁻¹ to about 4.0 h⁻¹, such as from about 1 h⁻¹ to about 3 h⁻¹ with the H₂ gas at 100-800 Nm³/m³. The hydrocarbons may exit the hydrotreatment reactor 135 through line L10 and may be flowed to olefin-producing unit 105. At the olefin-producing unit 105, the hydrocarbons may be converted to ethylene under any suitable conditions such as those described above. Additionally, or alternatively, the hydrocarbons produced by the hydrotreatment may be collected as a desired product as these hydrocarbons may include isoparaffins.

Additionally, or alternatively, the isobutylene oligomer coproducts flowing through line L9 may enter a cracking reactor 150 through line L16, where they are cracked to form isobutylene. Cracking, in general, breaks down or cracks the isobutylene oligomer coproducts to isobutylene. At the cracking reactor 150, the isobutylene oligomer coproducts may be converted to isobutylene under cracking conditions and in the presence of a cracking catalyst. Suitable catalysts for the cracking operation may include metal oxides, such as gamma-alumina; silica-alumina; activated metal oxides, such as solid BF₃ metal oxide complexes; zeolites, such as Y-zeolites; activated zeolites; or combinations thereof. Any suitable cracking conditions may be utilized such as a temperature in a range from about 400° C. to about 600° C., such as from about 450° C. to about 550° C., such as from about 475° C. to about 525° C., or from about 250° C. to about 450° C., such as from about 300° C. to about 400° C. Cracking conditions may include a pressure of about atmospheric pressure. The cracking conditions may include an LHSV that may be in a range from about 1 h⁻¹ to about 5 h⁻¹. The stream containing isobutylene oligomer coproducts may be diluted with an inert gas such as nitrogen to a volume percent of from about 10 vol % to about 90 vol %.

The cracking converts at least a portion of isobutylene oligomer coproducts entering the cracking reactor 150 to isobutylene. A % conversion for the cracking may be about 50% or more, from about 75% to about 100%, such as in a range from about 85% to about 95%, or greater than about 90%, such as about 91%, such as about 92%, such as about 93%, such as about 94%, such as about 95%, such as about 96%, such as about 97%, such as about 98%, such as about 99%, such as about 100% based on an amount of isobutylene oligomer coproducts entering the cracking reactor 150 through line L16.

The isobutylene collected from the cracking reactor 150 may include high purity isobutylene, for example, an HR-PIB grade isobutylene. The isobutylene formed by cracking may be collected for use as a feed for PIB production (for example, recycling to the polymerization unit 130 through line L17 and line L7), may be collected and used in processes for forming other high-value chemicals, or combinations thereof. A non-limiting result of cracking the isobutylene oligomer coproducts is to further increase the yield of isobutylene and PIB using embodiments described herein.

Referring back to the ethylene flowing through line L2, at least a portion of the ethylene stream exiting the olefin-producing unit 105 may be optionally flowed to metathesis reactor 145 through line L13. At least a portion of the 2-butene separated from the isomerization product effluent (described above) may also be fed to the metathesis reactor 145 through line L14. At the metathesis reactor 145, a mixture that includes a metathesis catalyst, the 2-butene, and the ethylene may be reacted, under metathesis conditions, in the metathesis reactor 145 to form propylene (according to the reaction: 2-butene+ethylene→propylene). Any suitable metathesis catalyst may be used. For example, the metathesis catalyst may include metals such as tungsten (W), molybdenum (Mo), rhenium (Re), or combinations thereof on a support such as, for example, silica (SiO₂) or alumina (Al2O₃). Additionally, or alternatively, metathesis catalysts may include a heterogeneous metathesis catalyst, a homogeneous catalyst, or combinations thereof. Suitable heterogeneous metathesis catalysts may include a metal oxide of W, Mo, or Re, such as Re₂O₇/Al₂O₃, WO₃/SiO₂, MoO₃/Al₂O₃, or combinations thereof. Suitable homogeneous metathesis catalysts may include a molybdenum alkylidene catalyst (for example, molybdenum imido alkylidene catalyst), a rhenium alkylidene catalyst, or combinations thereof, or combinations thereof. Suitable metathesis conditions to form the propylene may include a temperature in a range from about 200° C. to about 350° C., such as from about 250° C. to about 300° C.; a pressure in a range from about 2 MPa to about 5 MPa, such as from about 3 MPa to about 4 MPa; or combinations thereof. The resulting propylene from the metathesis may be recovered through line L15.

The system 100 of FIG. 1 may include a controller 160. Although not shown, the controller 160 may be coupled to the various elements (e.g., units, reactors, lines, etc.) of system 100 by use of a suitable line or appropriate wiring. The controller 160 is described below.

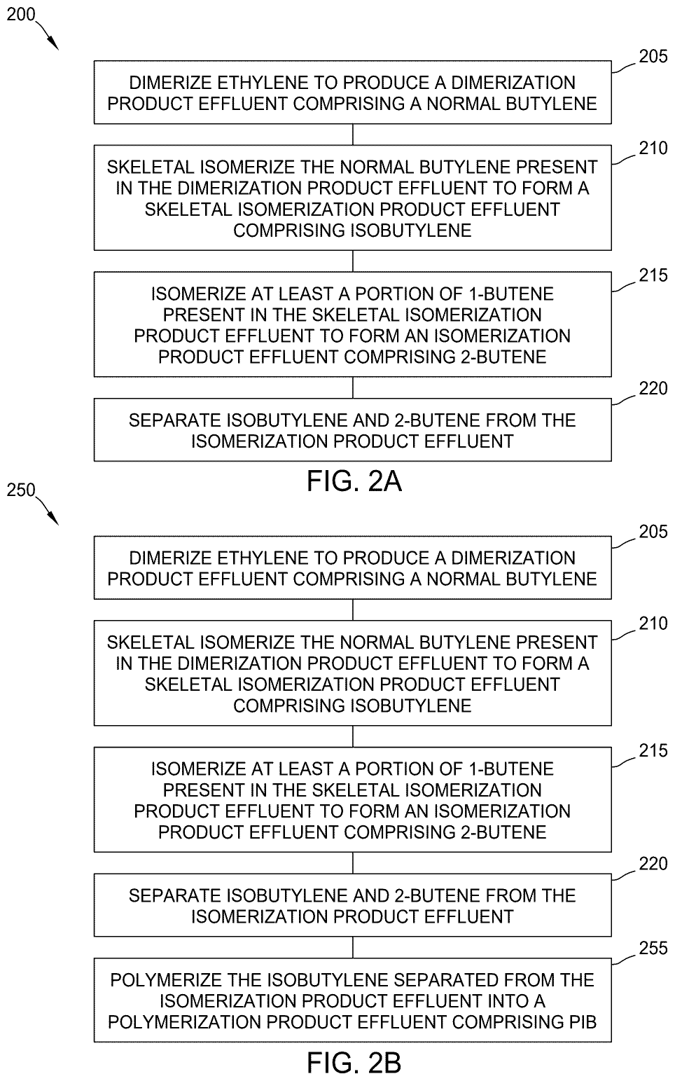

FIG. 2A is a flow diagram showing selected operations of a process 200 for forming isobutylene according to at least one embodiment of the present disclosure. The process 200 may be performed using system 100. In some embodiments, which may be combined with other embodiments, process 200 provides an implementation of producing isobutylene from ethylene by dimerization, skeletal isomerization, and isomerization of 1-butene to 2-butene to facilitate distillation. As described further herein, the isobutylene may be polymerized into PIB, such as HR-PIB.

In some embodiments, which may be combined with other embodiments, process 200 (and system 100) may be implemented as a closed skeletal isomerization-isomerization-separation recycle loop (for example, operation 210, operation 215, and operation 220) utilizing skeletal isomerization reactor 115, isomerization reactor 120, and separation unit 125. This recycle loop (allowing the materials traveling through the system to make more than one pass) may facilitate conversion of all or nearly all of the normal butylenes in C4-containing feeds (e.g., CC4 and/or dimerization product effluent) to be converted into isobutylene. In some embodiments, which may be combined with other embodiments, process 200 (and system 100) may be implemented as an open-loop, single pass arrangement where the materials undergo skeletal isomerization, isomerization, and separation one time.

The process 200 may begin with dimerizing ethylene to produce a dimerization product effluent comprising a normal butylene, for example, 1-butene, cis-2-butene, trans-2-butene, or combinations thereof, at operation 205. The dimerization process of operation 205 may be performed in the ethylene dimerization reactor 110. Here, for example, ethylene may be fed through line L2 to the ethylene dimerization reactor 110. At the ethylene dimerization reactor 110, the ethylene may contact the dimerization catalyst, under dimerization conditions, to convert the ethylene to 1-butene, cis-2-butene, trans-2-butene, or combinations thereof. Optionally, some amount of isobutylene may be formed by the dimerization. Suitable dimerization conditions for operating the ethylene dimerization reactor 110, dimerization catalysts, and ethylene feeds are described herein. The dimerization product effluent may then be discharged from the ethylene dimerization reactor 110 through line L3.

Additionally, or alternatively, normal butylenes may be sourced from other processes or streams such as a CC4 stream, an effluent from n-butane dehydrogenation, an effluent from n-butane dehydroisomerization, an effluent from isobutane dehydrogenation, or combinations thereof.

The process 200 may further include skeletal isomerizing the normal butylene present in the dimerization product effluent to form a skeletal isomerization product effluent comprising isobutylene at operation 210. This skeletal isomerization at operation 210 increases the yield of isobutylene, useful for the downstream polymerization process. Here, for example, the dimerization product effluent comprising 1-butene, cis-2-butene, trans-2-butene, or combinations thereof may be fed through line L3 to the skeletal isomerization reactor 115. At the skeletal isomerization reactor 115, 2-butene may be converted to isobutylene under skeletal isomerization conditions and in the presence of a skeletal isomerization catalyst. Suitable skeletal isomerization conditions for operating the skeletal isomerization reactor 115 and skeletal isomerization catalysts are described herein. The skeletal isomerization may also convert 1-butene to isobutylene under skeletal isomerization conditions and in the presence of the skeletal isomerization catalyst. As a result of the skeletal isomerization process, the skeletal isomerization product effluent exiting the skeletal isomerization reactor 115 through line L4 may have a concentration of isobutylene that is higher than a concentration of isobutylene entering the skeletal isomerization reactor 115 through line L3. The skeletal isomerization product effluent may then be discharged from the skeletal isomerization reactor 115 through line L4. The skeletal isomerization product effluent includes isobutylene, and may optionally include 1-butene and/or 2-butene.

The process 200 may further include isomerizing at least a portion of 1-butene present in the skeletal isomerization product effluent to form an isomerization product effluent comprising 2-butene at operation 215. Here, for example, the skeletal isomerization product effluent enriched in isobutylene may be fed through line L4 to the isomerization reactor 120. At the isomerization reactor 120, at least a portion of the 1-butene present in the isomerization product effluent may be converted to 2-butene under isomerization conditions and in the presence of an isomerization catalyst. Suitable isomerization conditions for operating the isomerization reactor 120 and isomerization catalysts are described herein. As described herein, an objective of the isomerization process of operation 215 is to facilitate and/or enhance downstream separation of isobutylene from normal butylenes present in the isomerization product effluent. The isomerization product effluent is enriched in 2-butene. Additionally, or alternatively, the isomerization product effluent exiting the isomerization reactor 120 through line L5 may have a concentration of 1-butene that is less than a concentration of 1-butene present in the skeletal isomerization product effluent entering the isomerization reactor 120 through line L4. The isomerization product effluent also includes isobutylene. The isomerization product effluent may then be discharged from the isomerization reactor 120 through line L5.

In some embodiments, which may be combined with other embodiments, the skeletal isomerization process of operation 210 and the isomerization process of operation 215 may be optionally performed in a single combination reactor. In these, and other embodiments, this optional operation may include one or more of the following: (a) feeding a 1-butene stream (for example, at least a portion of the dimerization product effluent) to the single combination reactor; (b) isomerizing 1-butene present in the dimerization product effluent to 2-butene in the single combination reactor; and (c) skeletal isomerizing 2-butene to isobutylene in the single combination reactor. The isomerization (b) may be performed before, during, or after the skeletal isomerization (c) because, for example, the 1-butene stream may include 2-butene. The isomerization (b) and skeletal isomerization (c) may be performed in the presence of an acidic catalyst and under the conditions described herein for operating the single combination reactor. The effluent exiting the single combination reactor after such an optional operation may include isobutylene and 2-butene, and may be substantially free of 1-butene as described herein. The effluent exiting the single combination reactor may then be processed in the same, or a similar, manner as that described herein with respect to separating isobutylene and 2-butene from the isomerization product effluent at operation 220.

The process 200 may further include separating isobutylene and 2-butene from the isomerization product effluent at operation 220. Here, for example, the isomerization product effluent comprising isobutylene and 2-butene may be fed through line L5 to the separation unit 125. At the separation unit 125, the isomerization product effluent may be subjected to separation conditions effective to separate isobutylene and 2-butene from the isomerization product effluent. The separation conditions may also be effective to separate the isobutylene from the 2-butene. The separation conditions may also be effective to separate 1-butene from isobutylene. Separation conditions and apparatus/techniques for separation at the separation unit 125 are described herein. The isobutylene (or isobutylene fraction) may then be discharged from the separation unit 125, for example, leaving “overhead” through line L7. The 2-butene (or 2-butene fraction) may also be discharged from the separation unit 125, for example, leaving as “bottoms” through line L6.

Additionally, or alternatively, separation of isobutylene and 2-butene, and optionally 1-butene, from the isomerization product effluent at operation 220 may involve: (i) an adsorptive separation (for example, molecular sieves or zeolites selective for olefin/paraffin separations); (ii) an extractive distillation using selective solvents (for example, solvents such as n-methylpyrrolidone (NMP), furfural, acetonitrile, or sulfolane); and/or (iii) a reactive conversion approach where 1-butene is shifted into a different C4 olefin form prior to separation (for example, reactive distillation to convert 1-butene to 2-butene utilizing a separation unit configured as a reactive distillation unit incorporating an acidic isomerization catalyst).

Process 200 may optionally include skeletal isomerizing the 2-butene separated to form isobutylene. Here, for example, the 2-butene fraction exiting the separation unit 125 through line L6 and may be flowed to the skeletal isomerization reactor 115. The skeletal isomerization converts the 2-butene to isobutylene, under skeletal isomerization conditions. This optional skeletal isomerization process may be performed in the same or a similar manner as that of operation 210. This optional recycling of the 2-butene back to the skeletal isomerization reactor 115 for skeletal isomerization may further increase the yield of isobutylene useful for the downstream polymerization process.

Prior to dimerizing ethylene to produce the dimerization product effluent, process 200 may further optionally include converting light hydrocarbons into ethylene. Here, for example, light hydrocarbons (such as C2-C6 hydrocarbons, such as C2-C4 hydrocarbons) may be fed through line L1 into olefin-producing unit 105. The olefin-producing unit 105 may include any suitable apparatus to form ethylene such as, for example, a steam cracker or a dehydrogenation reactor. At the olefin-producing unit 105, the light hydrocarbons may be converted under olefin-producing conditions effective to form ethylene. Olefin-producing conditions and the olefin-producing unit 105 are described herein. An olefin-containing effluent may then be discharged from the olefin-producing unit 105 through line L2. The olefin-containing effluent (also referred to as an ethylene stream) may then be fed to the ethylene dimerization reactor 110 where the ethylene is dimerized as described herein. Optionally, ethylene may be separated from the olefin-containing effluent in the olefin-producing unit 105 and then discharged from the olefin-producing unit 105 through line L2 prior to ethylene dimerization.

The process 200 may further optionally include an optional metathesis operation, whereby propylene is formed in the metathesis reactor 145 from 2-butene and ethylene. Here, at least a portion of the 2-butene separated from the isomerization product effluent may be fed from the separation unit 125 to the metathesis reactor 145 through line L6 and line L14. At least a portion of an ethylene stream produced at the olefin-producing unit 105 may also be fed from the olefin-producing unit 105 to the metathesis reactor through line L2 and line L13. At the metathesis reactor 145, a mixture that includes a 2-butene (or 2-butene fraction) separated from the isomerization product effluent and the ethylene stream may be formed by combining the two feeds. The mixture may contact the metathesis catalyst, under metathesis conditions, in the metathesis reactor 145 to form propylene. Suitable metathesis catalysts and metathesis conditions are described herein. Here, the metathesis reaction includes 2-butene reacting with ethylene to form two molecules of propylene. The reaction product mixture comprising the desired propylene may further include unreacted 2-butene. The propylene may be separated from the reaction product mixture to form a propylene stream. The resulting propylene stream may be discharged from the metathesis reactor 145 through line L15. This propylene stream is a valuable product stream. Use of the optional metathesis operation may further improve process integration and carbon efficiency of the system 100. Overall, the optional metathesis operation may provide a practical outlet for surplus 2-butene, improve overall carbon efficiency, and/or generate on-purpose propylene, aligning the process with modern petrochemical integration strategies and market demand.

The process 200 may further optionally include processing of the unreacted 2-butene (if any) present in the reaction product mixture after the optional metathesis operation. Such processing of the unreacted 2-butene may include recycling the unreacted 2-butene back to the metathesis reactor 145. Such recycling may facilitate the conversion efficiency of the metathesis. Additionally, or alternatively, processing of the unreacted 2-butene may include skeletal isomerizing the unreacted 2-butene to form isobutylene. Here, the unreacted 2-butene may be flowed to the skeletal isomerization reactor 115 where it may be converted to isobutylene as described herein, for example, with respect to operation 210. Suitable skeletal isomerization conditions for operating the skeletal isomerization reactor 115 and skeletal isomerization catalysts are described herein.

The process 200 may further optionally include converting isobutylene oligomer coproducts into an olefin-containing effluent comprising ethylene. As described herein, isobutylene oligomer coproducts may be formed during isobutylene polymerization at the polymerization unit 130. The isobutylene oligomer coproducts may be converted into ethylene by, for example, by: (a) hydrotreating the isobutylene oligomer coproducts to form hydrotreated C8-C20 hydrocarbons; and (b) converting these hydrotreated C8-C20 hydrocarbons to ethylene. Here, isobutylene oligomer coproducts may be fed through line L9 to hydrotreatment reactor 135, where the isobutylene oligomer coproducts are converted, under hydrotreatment conditions and in the presence of a hydrotreatment catalyst, to the hydrotreated C8-C20 hydrocarbons (for example, hydrotreated C8-C16 hydrocarbons, such as a hydrotreated C8 hydrocarbon, a hydrotreated C12 hydrocarbon, a hydrotreated C16 hydrocarbon, or combinations thereof). Suitable hydrotreatment conditions and hydrotreatment catalysts are described herein. The hydrocarbons may then be discharged from the hydrotreatment reactor 135 through line L10 and fed to olefin-producing unit 105. At the olefin-producing unit 105, the hydrocarbons may be converted to ethylene under olefin-producing conditions. Suitable olefin-producing conditions to form ethylene and are described herein. An olefin-containing effluent comprising the ethylene is produced from the conversion. The olefin-containing effluent may optionally include CC4. In such cases, ethylene and/or CC4 (in separate fractions) may be separated from the olefin-containing effluent by performing, for example, a distillation, a fractional distillation, a vacuum distillation, a flash evaporation, a fractionation, an extraction, a decantation, a coalescence, or combinations thereof, on the olefin-containing effluent. This separation of ethylene and/or CC4 from the olefin-containing effluent may be performed at olefin-producing unit 105. The ethylene, or olefin-containing effluent comprising ethylene, may be discharged from the olefin-producing unit 105 through line L2 or a separate line.

FIG. 2B shows selected operations of a process 250 for forming PIB according to at least one embodiment of the present disclosure. The process 250 may be performed using system 100. In some embodiments, which may be combined with other embodiments, process 250 provides an implementation of producing PIB from ethylene that includes dimerizing ethylene to produce 1-butene and 2-butene, followed by skeletal isomerization to convert at least a portion of these linear butylenes into isobutylene, separating the isobutylene, and polymerizing the isobutylene into PIB, such as HR-PIB.

The process 250 may include operation 205 (dimerization), operation 210 (skeletal isomerization), operation 215 (isomerization), and operation 220 (separation) described herein with respect to process 200. The process 250 may optionally further include one or more of those optional operations described herein with respect to process 200. After operation 220, isobutylene (or an isobutylene fraction) flows through line L7.

The process 250 may further include polymerizing the isobutylene separated from the isomerization product into a polymerization product effluent comprising PIB at operation 255. Here, the isobutylene flowing in line L7 may be fed to a polymerization reactor of the polymerization unit 130. In the polymerization reactor, a polymerization reaction mixture that includes isobutylene and polymerization catalyst may be formed. The mixture may be reacted, under polymerization conditions, in the polymerization unit to convert the isobutylene to the polyisobutylene. During the polymerization, the isobutylene may contact the polymerization catalyst, under the polymerization conditions, to convert the isobutylene to PIB, such as HR-PIB.

Suitable polymerization catalysts are described herein. As an illustrative, but non-limiting example, the polymerization catalyst may include BF₃. The polymerization catalyst may include a solid substrate (for example, silica-alumina) comprising sorbed BF₃ catalyst complex. The BF₃ catalyst complex may include BF₃ and a complexing agent comprising an oxygen-containing compound, such as an oxygen-containing compound that is free of beta-hydrogen atoms. The solid substrate comprising sorbed BF₃ catalyst complex (for example, BF₃·MeOH) may include any suitable amount of BF₃, for example, about 30 wt % or more of BF₃ based on the total wt % of the solid substrate comprising sorbed catalyst complex. Other suitable polymerization catalysts are contemplated.

Suitable polymerization conditions are described herein. As an illustrative, but non-limiting example, polymerization conditions may include a temperature in a range from about −10° C. to about 40° C.; a polymerization period/reaction time of about 4 minutes or less; or combinations thereof. Other suitable polymerization conditions are contemplated including those described herein. The polymerization reactor may include a tubular loop reactor. As described herein, tubular loop reactors facilitate high-velocity movement of the polymerization reaction mixture (the mixture comprising isobutylene and polymerization catalyst). The high-velocity reaction mixture may improve heat transfer/removal and catalyst dispersion. The heat transfer/removal and catalyst dispersion may help facilitate control (or precise control) of molecular weight distribution and/or may help facilitate high selectivity for terminal double bonds in the PIB product (that is, high selectivity for HR-PIB, such as HR-PIB having an alpha vinylidene content greater than 75 wt %). The controlled reaction conditions and advanced reactor design may help ensure a consistent product with a molecular weight distribution suitable for industrial applications such as fuel and lubricant additives, sealants, and adhesives, among other applications. Other suitable polymerization reactors, polymerization conditions, etc. are contemplated.

In some examples, this polymerization catalyst/polymerization reactor system may achieve greater than 80% alpha vinylidene content (for example, greater than 90% alpha vinylidene content) and narrow molecular weight distributions.

As described herein, the polymerization product effluent that includes the PIB, such as HR-PIB, may include one or more optional components such as isobutylene oligomer coproducts, unreacted isobutylene, hydrocarbon diluent, polymerization catalyst residues, or combinations thereof. The one or more optional components of the polymerization product effluent may be separated from the polymerization product effluent and/or may be recycled to various units in the system 100. As an example, the process 250 may optionally include separating isobutylene oligomer coproducts from the polymerization product effluent by use of an oligomer separation unit in the polymerization unit 130. Here, the isobutylene oligomer coproducts may be separated by, for example, distillation as described herein whereby the isobutylene oligomer coproducts may be discharged from the polymerization unit 130 (e.g., leaving “overhead”) through line L9 and the PIB may be discharged from the polymerization unit 130 through line L8 (e.g., leaving as a bottoms stream).

The isobutylene oligomer coproducts flowing through line L9 may then be subjected to further processing, such as cracking, hydrotreatment, or combinations thereof.

For example, the process 250 may further optionally include converting the isobutylene oligomer coproducts to isobutylene by cracking (e.g., thermal cracking). Here, the isobutylene oligomer coproducts may be fed to the cracking reactor 150 through line L9 and L16. At the cracking reactor 150, the isobutylene oligomer coproducts may contact a cracking catalyst, under cracking conditions, to convert the isobutylene oligomer coproducts to isobutylene. Suitable cracking conditions and cracking catalysts are described herein.

The cracked isobutylene (the isobutylene produced by cracking the isobutylene oligomer coproducts) may be recycled back into the polymerization reactor of the polymerization unit 130. For example, the isobutylene may be discharged from the cracking reactor 150 through line L17 and may be fed to the isobutylene flowing through line L7 and into the polymerization unit 130, where the isobutylene may be polymerized in the polymerization reactor. Overall, the optional cracking operation on the isobutylene oligomer coproducts and subsequent polymerization of the isobutylene may help minimize waste, may help enhance feedstock utilization, and may address potential (if any) selectivity losses from the oligomer formation by converting the isobutylene oligomer coproducts into useful intermediates.

Additionally, or alternatively, the process 250 may further optionally include hydrotreating the isobutylene oligomer coproducts in hydrotreatment reactor 135. The isobutylene oligomer coproducts may be fed to the hydrotreatment reactor 135 through line L9. The hydrotreatment converts the isobutylene oligomer coproducts to hydrotreated C8-C20 hydrocarbons. As a recycled feedstock, the hydrotreated C8-C20 hydrocarbons (exiting the hydrotreatment reactor 135 through line L10) may then be processed in an olefin-producing unit 105 (for example, a steam cracker) to produce additional ethylene and/or other valuable products, closing the loop on waste and improving the overall efficiency of the process. For example, at the olefin-producing unit 105, hydrocarbons fed from the hydrotreatment reactor 135 and into the olefin-producing unit 105 may be converted to ethylene, propylene, or combinations thereof. This optional hydrotreatment and conversion to ethylene is described herein with respect to process 200.

The ability to subject the isobutylene oligomer coproducts flowing through line L9 to further processing, such as cracking, hydrotreatment, or combinations thereof may facilitate a sustainable, low-waste design in which all (or substantially all) carbon either enters PIB production or is returned to the front end of the process for reuse.

The process 250 may further optionally include hydrogenating the PIB (for example, HR-PIB) flowing through line L8. Here, the PIB flowing through line L8 may be fed to a hydrogenation reactor (not shown in FIG. 1). At the hydrogenation reactor, H₂ gas may contact a mixture comprising PIB and a hydrogenation catalyst, under hydrogenation conditions, to convert the PIB into the hydrogenated PIB (such as hydrogenated HR-PIB). Suitable hydrogenation catalysts may include Ni—Mo, Co—Mo, Pd/C, Pt/C, Ru/C, Raney Ni, or combinations thereof. The hydrogenation reactor may be operated under any suitable hydrogenation conditions to form hydrogenated PIB. Suitable hydrogenation conditions may include a temperature in a range from about 150° C. to about 350° C., such as from about a temperature in a range from about 150° C. to about 250° C.; a pressure in a range from about 2 MPa to about 8 MPa, such as from about 3 MPa to about 8 MPa, such as from about 4 MPa to about 6 MPa; or a combination thereof. H₂ gas may be fed to the hydrogenation reactor at any suitable pressure, such as a pressure in a range from about 0.5 MPa to about 10 MPa, such as from about 1 MPa to about 8 MPa, depending on, for example, the catalyst utilized. The hydrogenated PIB (for example, hydrogenated HR-PIB) may then be discharged from the hydrogenation reactor.

The hydrogenated PIB may include a partially unsaturated PIB, such as a partially unsaturated HR-PIB. The partially unsaturated PIB retains some double bonds for downstream functionalization. Additionally, or alternatively, the hydrogenated PIB may include a fully saturated PIB, such as a fully saturated HR-PIB. The fully saturated PIB is completely hydrogenated (has no double bonds) and non-reactive. Fully saturated PIB may be useful in high value applications such as cosmetics and personal care products, as well as specialty lubricants, among other applications. HR-PIB (not hydrogenated) is useful as, for example, fuel and lubricant additives, sealants, and adhesives, among other applications. By incorporating hydrogenation of the PIB as an optional process operation on the PIB, the optional hydrogenated PIB is formed that may be used for various and/or distinct applications as described herein.

In some implementations, which may be combined with other implementations, embodiments described herein may include one or more of the following. A light hydrocarbon stream, such as a C2-C4 hydrocarbon stream such as ethane, propane, butane, or combinations thereof, such as ethane, may be introduced into an olefin-producing unit 105 (for example, a steam cracker), where ethylene is produced. This ethylene may then enter ethylene dimerization reactor 110, yielding a mixture of C4 olefins, such as 1-butene and 2-butene, and optionally a small amount of isobutylene. The mixed C4 stream (dimerization product effluent) may be directed into skeletal isomerization reactor 115, where the C4 olefins may be predominantly converted into isobutylene, and optionally some 1-butene and 2-butene. The output from the skeletal isomerization reactor 115 may then be sent to an isomerization reactor 120 to convert 1-butene into 2-butene, aiding in the separation of isobutylene. Next, the isomerized product may be flowed into separation unit 125 (e.g., a fractional distillation column), where isobutylene may be taken overhead and may then be polymerized at polymerization unit 130 to PIB, such as HR-PIB, and optionally a small amount of isobutylene oligomer coproducts. The 2-butene remaining at the bottom of the separation unit 125 (e.g., a fractional distillation column) may be recycled back to the skeletal isomerization reactor 115, while the isobutylene oligomer coproducts from the PIB polymerization process may be hydrotreated and recycled into the steam cracker (and/or other olefin-producing unit 105).

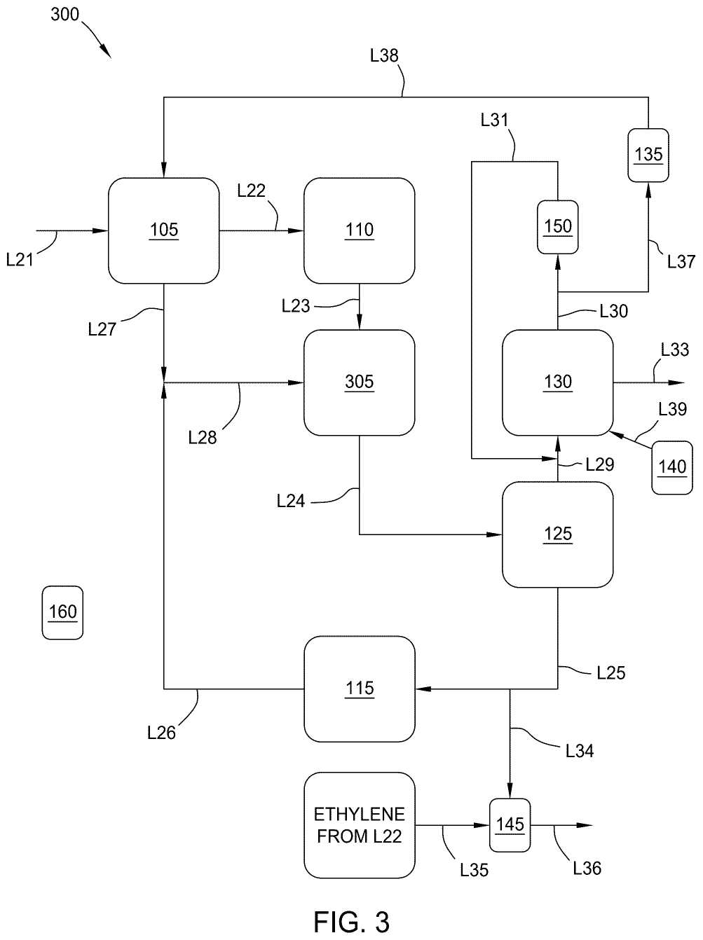

FIG. 3 is a generalized schematic flow diagram showing various embodiments of processes described herein corresponding to operational areas or units in a system 300 (for example, a processing plant) for forming isobutylene and PIB according to at least one embodiment of the present disclosure. The system 300 includes embodiments for isobutylene production and PIB (for example, HR-PIB) production. The system 300 may be run in a batch or a continuous process. System 300 may incorporate various units, reactors, and operations described herein with respect to system 100 of FIG. 1.

In some embodiments, which may be combined with other embodiments, the system 300 may facilitate, for example, ethylene dimerization to be advantageously integrated with the conversion of the crude C4 (CC4) stream from an olefin-producing unit 105 (for example, a steam cracker). Here, the CC4 stream may be processed alongside the dimerized ethylene stream to maximize isobutylene production. The isobutylene may be fed into a polymerization reactor to form PIB, such as HR-PIB. In some implementations, the CC4 and ethylene may, but do not have to, originate from the same olefin-producing unit 105. Instead of CC4, any suitable C4-containing feed may be utilized.

A light hydrocarbon (LHC) stream may enter the system 300 through line L21 and enters an olefin-producing unit 105 such as a steam cracker or a dehydrogenation reactor. The light hydrocarbon stream may include a C2-C6 hydrocarbon stream, such as a C2-C4 hydrocarbon stream, such as ethane, propane, butane, or combinations thereof, such as ethane. Processing of the light hydrocarbons in the olefin-producing unit 105 is described herein. Additionally, or alternatively, an ethylene stream may enter the system 300 through line L22 and enter the ethylene dimerization reactor 110 such that the olefin-producing unit 105 is optional.

The olefin-containing effluent comprising ethylene may also include CC4. When ethane is cracked, the CC4 may be produced at a level equal to about 3-5% of the ethylene produced and may include 1,3-butadiene, 1-butene, and/or 2-butene, and optionally smaller amounts of isobutylene. Ethylene and the CC4 may be separated by any suitable separation technique. For example, suitable separation techniques may include performing a distillation, a fractional distillation, a vacuum distillation, a flash evaporation, a fractionation, an extraction, a decantation, a coalescence, or combinations thereof, on the olefin-containing effluent. This separation may be performed at the olefin-producing unit 105.

The olefin-containing effluent comprising ethylene may exit the olefin-producing unit 105 through line L22 and may be flowed to an ethylene dimerization reactor 110. Processing of the ethylene in the ethylene dimerization reactor is described herein. A dimerization product effluent may be made at the ethylene dimerization reactor. The dimerization product effluent (a C4-containing feed) may include a normal butylene, for example, 1-butene, cis-2-butene, trans-2-butene, or combinations thereof, and optionally minor amounts of isobutylene.

The dimerization product effluent may exit the ethylene dimerization reactor 110 through line L23 and may be flowed to a selective hydrogenation-isomerization unit (SHU) 305. The CC4 (a C4-containing feed) may exit the olefin-producing unit 105 and may be flowed to the SHU 305 through line L27 and line L28. Additionally, or alternatively, CC4 may be sourced from an effluent from n-butane dehydrogenation, an effluent from n-butane dehydroisomerization, an effluent from isobutane dehydrogenation, or combinations thereof, among other sources.

The SHU 305 is utilized to perform a selective hydrogenation-isomerization reaction. For example, 1,3-butadiene present in the combined CC4 and dimerization product effluent may be selectively hydrogenated into 1-butene. Simultaneously or sequentially, the 1-butene formed may be isomerized into 2-butene. In addition, 1-butene present in the combined CC4 and dimerization product effluent may be isomerized into 2-butene. The isomerization of 1-butene to 2-butene at the SHU 305 facilitates downstream separation of isobutylene from normal butylenes (for example, in separation unit 125) by advantageously utilizing the boiling point differences. As described herein, 2-butene has a significantly higher boiling point than isobutylene (a difference of about 7° C. to about 11° C.), while 1-butene has a very close boiling point to that of isobutylene (a difference of less than 1° C.). With the isomerization of 1-butene to 2-butene at SHU 305, at least a portion of 1-butene is converted to 2-butene, facilitating separation.

The selective hydrogenation-isomerization reaction may be performed under selective hydrogenation-isomerization conditions and in the presence of a selective hydrogenation-isomerization catalyst. Any suitable selective hydrogenation-isomerization catalyst may be used. For example, the selective hydrogenation-isomerization catalyst may include palladium (Pd) metal adsorbed on a substrate, where the substrate may be a metal oxide such as alumina or gamma alumina, at a Pd concentration in a range from about 0.1 wt % to about 10 wt %, such as from about 0.5 wt % to about 5 wt %, such as from about 1.0 wt % to about 2 wt %. Optionally, the Pd catalyst may be sulfurized to further improve selectivity and reduce the possibility of over-hydrogenation. The operating conditions (selective hydrogenation-isomerization conditions) may be selected so that the reactants and products may be at least partially in the liquid phase. It may, however, be advantageous to select an operating mode such that the products may be partially vaporized at an outlet of the SHU 305, to facilitate thermal control of the reaction. Selective hydrogenation-isomerization conditions may include a temperature in a range from about 50° C. to about 150° C., such as from about 60° C. to about 150° C., such as from about 75° C. to about 125° C., such as from about 95° C. to about 105° C.; a pressure in a range from about 0.3 MPa to about 4.1 MPa, such as from about 0.3 MPa to about 3.4 MPa, such as from about 1.0 MPa to about 3.0 MPa, such as from about 1.5 MPa to about 2.5 MPa, such as from about 1.75 MPa to about 2.25 MPa; a space velocity in a range from about 0.5 h⁻¹ to about 10 h⁻¹, such as from about 1 h⁻¹ to about 6 h⁻¹; or combinations thereof. H₂ gas may be fed to the selective hydrogenation-isomerization reactor at any suitable pressure, such as a pressure in a range from about 0.5 MPa to about 5.0 MPa, such as from about 1.0 MPa to about 3.0 MPa, such as from about 1.5 MPa to about 2.5 MPa.

It may be beneficial to use the selective hydrogenation-isomerization catalyst in at least one downflow fixed catalyst bed reactor. When the proportion of 1,3-butadiene in the cut is high, as is the case, for example, for a steam cracking CC4 cut wherein the 1,3-butadiene is not to be extracted, it may be advantageous to effect the transformation in two reactors in series to better control the hydrogenation selectivity. The second reactor may be an upflow reactor and may act as a finisher. The quantity of hydrogen utilized for the reactions carried out in the selective hydrogenation-isomerization may be adjusted as a function of the composition of the cut so that, for example, there is only a slight excess of hydrogen with respect to the theoretical stoichiometry.

The selective hydrogenation-isomerization forms a selective hydrogenation-isomerization product effluent. Besides 2-butene, the selective hydrogenation-isomerization product effluent may include 1-butene, isobutylene, or combinations thereof. After the first round of processing through the SHU 305, separation unit 125, and skeletal isomerization reactor 115 of FIG. 3, the amount of isobutylene in the selective hydrogenation-isomerization product effluent increases. This increase in isobutylene may be a consequence of the 2-butene separated at separation unit 125 being converted to isobutylene at the skeletal isomerization reactor 115 and/or the 1-butene being converted to 2-butene and then converted to isobutylene as it travels through the SHU 305, separation unit 125, and skeletal isomerization reactor 115.

The selective hydrogenation-isomerization product effluent exiting the SHU 305 through line L24 may have a concentration of 2-butene that is greater than a concentration of 2-butene entering the SHU 305 through line L23 and/or line L28. Additionally, or alternatively, the selective hydrogenation-isomerization product effluent exiting the SHU 305 through line L24 may have a concentration of 1,3-butadiene that is less than a concentration of 1,3-butadiene entering the SHU 305 through line L23 and line L28. Additionally, or alternatively, the selective hydrogenation-isomerization product effluent exiting the SHU 305 through line L24 may have a concentration of 1-butene that is less than a concentration of 1-butene entering the SHU 305 through line L23 and line L28.

The selective hydrogenation-isomerization product effluent comprising 2-butene may be flowed to a separation unit 125 through line L4. Separation of the selective hydrogenation-isomerization product effluent in the separation unit 125 may be performed in the same or similar manner as the separation of the isomerization product effluent in the separation unit 125 described herein with respect to system 100 and process 200. Suitable apparatus include apparatus to perform a distillation, a vacuum distillation, a fractional distillation, a reactive distillation, a flash evaporation, a fractionation, an extraction, a decantation, a coalescence, or combinations thereof. For example, the separation unit 125 may include any suitable apparatus, such as a fractional distillation column, that separates the components (or fractions) by boiling points.

At the separation unit 125, the components of the selective hydrogenation-isomerization product effluent are separated. As described above, the concentration of isobutylene increases after the first round of processing. Therefore, the selective hydrogenation-isomerization product effluent may be separated into an isobutylene fraction (which may exit the separation unit 125 through line L29) and a 2-butene fraction (which may exit the separation unit 125 through line L25). The selective hydrogenation-isomerization product effluent may also include n-butane, isobutane, or combinations thereof. In such instances, the separation unit 125 may also separate an isobutane fraction and an n-butane fraction which may exit the separation unit through different lines (not shown).

The 2-butene fraction exiting the separation unit 125 through line L25 may be flowed to the skeletal isomerization reactor 115. The skeletal isomerization reactor 115 may be operated to perform a skeletal isomerization process (SKIP process). Skeletal isomerization may be performed as described herein, for example, with respect to embodiments of system 100, process 200, and/or process 250. At the skeletal isomerization reactor 115, at least a portion of the normal butylene (1-butene, trans-2-butene, cis-2-butene, or combinations thereof) may be skeletally isomerized, under skeletal isomerization conditions, to form a skeletal isomerization product effluent that is enriched in isobutylene. That is, the skeletal isomerization effluent exiting the skeletal isomerization reactor 115 through line L26 may have a higher concentration of isobutylene than the feed entering the skeletal isomerization reactor 115 through line L25. In addition to isobutylene, the skeletal isomerization effluent exiting the skeletal isomerization reactor 115 through line L26 may include 2-butene, 1-butene, or combinations thereof.

The skeletal isomerization product effluent flowing through line L26 may then be fed back to the SHU 305. As shown in FIG. 3, the skeletal isomerization effluent may be combined with the CC4 feed, and the combined feed may be fed to the SHU 305 through line L28. At the SHU, 1-butene present in the skeletal isomerization effluent is converted to 2-butene. Generally 305, isobutylene present in the skeletal isomerization effluent remains unchanged during the selective hydrogenation-isomerization process. The SHU 305 and its operation is described herein. The skeletal isomerization product effluent may then be fed to the separation unit 125 as described herein.

Overall, recycling of the 2-butene from the separation unit 125 to the skeletal isomerization reactor 115 and then to the SHU 305 further increases conversion of the dimerization product effluent and CC4 into isobutylene.

The isobutylene fraction (isobutylene feed), which may be high purity isobutylene, exiting the separation unit 125 through line L29 may be flowed to a polymerization reactor of the polymerization unit 130. Polymerization catalyst, held in polymerization catalyst unit 140, may be fed into the polymerization reactor of the polymerization unit 130 through line L39. The polymerization process at polymerization unit 130, as well as polymerization catalysts, polymerization reactors, polymerization conditions, etc. may be performed as described herein, for example, with respect to embodiments of system 100 and/or process 250. The polymerization forms PIB, such as HR-PIB. HR-PIB may exit the polymerization unit 130 through line L33.

Isobutylene oligomer coproducts may exit the polymerization unit 130 through line L30. The isobutylene oligomer coproducts may be fed to hydrotreatment reactor 135 through line L30 and L37. At the hydrotreatment reactor 135, the isobutylene oligomer coproducts may be converted to hydrocarbons under hydrotreatment conditions and in the presence of a hydrotreatment catalyst. The hydrocarbons produced from the hydrotreatment may include hydrotreated C8-C20 hydrocarbons, such as hydrotreated C8-C16 hydrocarbons, such as a hydrotreated C8 hydrocarbon, a hydrotreated C12 hydrocarbon, a hydrotreated C16 hydrocarbon, or combinations thereof. The hydrotreatment of isobutylene oligomer coproducts may be performed as described herein, for example, with respect to embodiments of system 100, process 200, and/or process 250. The hydrotreated hydrocarbons produced may then be fed to the olefin-producing unit 105 through line L38 where the hydrocarbons may be converted to ethylene. Processing of the hydrocarbons in the olefin-producing unit 105 may be performed as described herein, for example, with respect to embodiments of system 100, process 200, and/or process 250. Additionally, or alternatively, the hydrocarbons produced by the hydrotreatment may be collected as a desired product as these hydrocarbons may include isoparaffins.

Additionally, or alternatively, the isobutylene oligomer coproducts may enter cracking reactor 150 through line L30, where they are cracked to form isobutylene. Processing of the isobutylene oligomer coproducts by cracking in the cracking reactor 150 may be performed as described herein, for example, with respect to embodiments of system 100, process 200, and/or process 250. The isobutylene formed at the cracking reactor (also referred to herein as cracked isobutylene) may then be further processed to form PIB as described herein.

In addition, propylene may optionally be formed by implementations of system 300. Here, for example, at least a portion of the ethylene flowing through line L22 may be flowed to metathesis reactor 145 through line L35 and at least a portion of the 2-butene flowing through line L25 may be flowed to metathesis reactor 145 through line L34. The 2-butene and ethylene may then be reacted to form propylene. The metathesis to form propylene may be performed as described herein, for example, with respect to embodiments of system 100, process 200, and/or process 250.

The system 300 may include a controller 160. Although not shown, the controller 160 may be coupled to the various elements (e.g., units, reactors, lines, etc.) of system 300 by use of a suitable line or appropriate wiring. The controller 160 is described below.

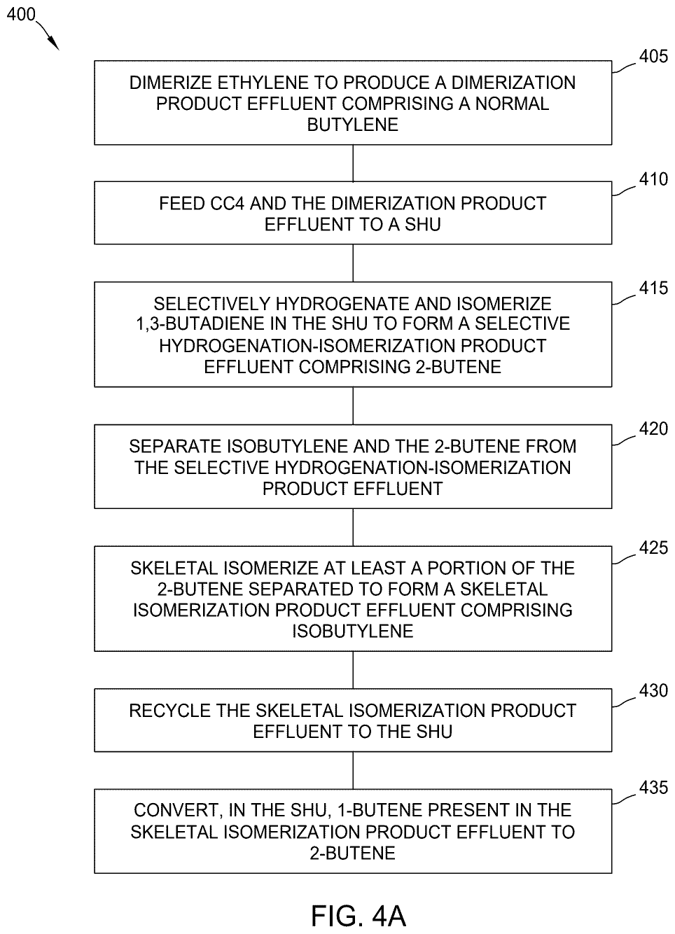

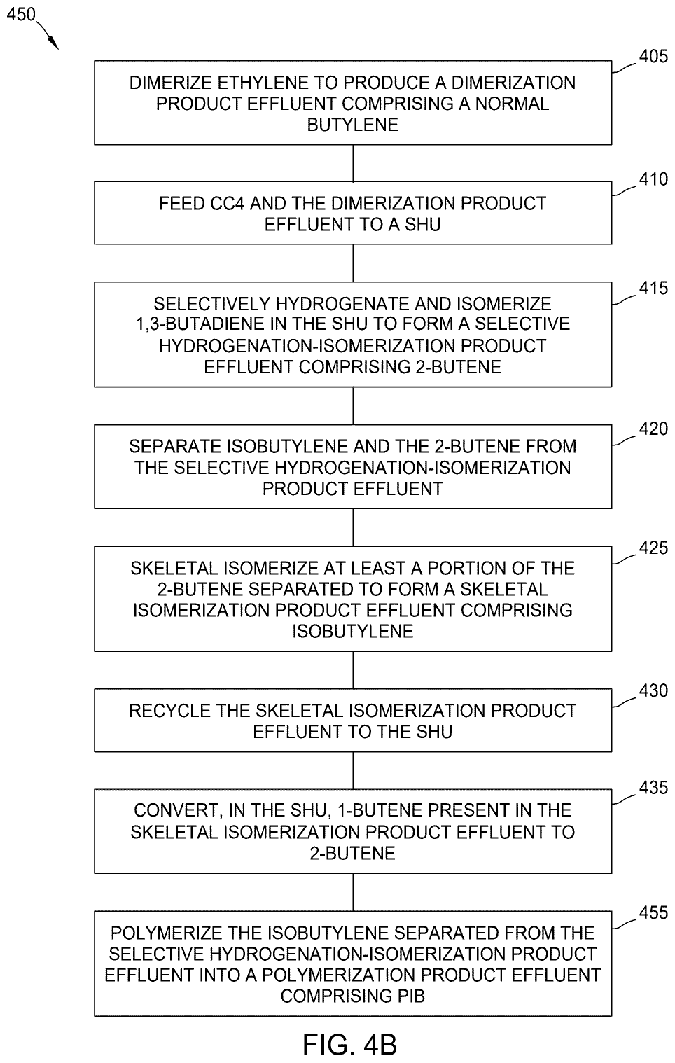

FIG. 4A is a flow diagram showing selected operations of a process 400 for forming isobutylene according to at least one embodiment of the present disclosure. The process 400 may be performed using system 300. In some embodiments, which may be combined with other embodiments, process 400 provides an implementation of producing isobutylene, wherein ethylene dimerization product effluent and CC4 may be processed to enhance isobutylene production, including integration with selective hydrogenation-isomerization and recycling of 2-butene for skeletal isomerization. Process 400 may incorporate various operations described herein with respect to process 200 of FIG. 2A.

In some embodiments, which may be combined with other embodiments, process 400 (and system 300) may be implemented as a closed SHU-Fractionation-SKIP recycle loop (for example, operations 410 through 435) utilizing SHU 305, separation unit 125, and skeletal isomerization reactor 115. This recycle loop (allowing the materials traveling through the system to make more than one pass) may facilitate conversion of all or nearly all of the normal butylenes in C4-containing feeds (e.g., CC4 and/or dimerization product effluent) to be converted into isobutylene. In some embodiments, which may be combined with other embodiments, process 400 (and system 300) may be implemented as an open-loop, single pass arrangement where the materials undergo skeletal isomerization, isomerization, and separation one time. For example, a stream may travel through the SHU 305, separation unit 125, and skeletal isomerization reactor 115 in one pass, and in the following final pass, the stream passes through only the SHU 305 and the separation unit 125.

The process 400 may begin with dimerizing ethylene to produce a dimerization product effluent comprising a normal butylene, for example, 1-butene, cis-2-butene, trans-2-butene, or combinations thereof, at operation 405. The dimerization process of operation 405 may be performed in the ethylene dimerization reactor 110. Here, for example, ethylene may be fed through line L22 to the ethylene dimerization reactor 110. At the ethylene dimerization reactor 110, the ethylene may contact the dimerization catalyst, under dimerization conditions, to convert the ethylene to normal butylenes (for example, 1-butene, cis-2-butene, trans-2-butene, or combinations thereof). Optionally, some amount of isobutylene may be formed by the dimerization. Suitable dimerization conditions for operating the ethylene dimerization reactor 110, dimerization catalysts, and ethylene feeds are described herein. The dimerization product effluent may then be discharged from the ethylene dimerization reactor 110 through line L23. Additionally, or alternatively, normal butylenes may be sourced from an effluent from n-butane dehydrogenation, an effluent from n-butane dehydroisomerization, an effluent from isobutane dehydrogenation, or combinations thereof. The normal butylenes may be fed to the SHU 305 with or without the dimerization product effluent.

The process 400 may further include feeding CC4 (through line L27 and L28) and the dimerized product effluent (through line L23) into the SHU 305 at operation 410. The CC4 may include 1,3-butadiene. The CC4 feed may further include 1-butene, cis-2-butene, trans-2-butene, or combinations thereof. Feeding at operation 410 may be simultaneous or consecutive. Additionally, or alternatively, the dimerized ethylene effluent may be combined with the CC4 prior to entering the SHU 305.

The process 400 may further include selectively hydrogenating and isomerizing the 1,3-butadiene in the SHU 305 to form a selective hydrogenation-isomerization product effluent comprising 2-butene at operation 415. The selective hydrogenation and isomerization process of operation 415 may include contacting 1,3-butadiene with a selective hydrogenation-isomerization catalyst to convert the 1,3-butadiene into 1-butene under selective hydrogenation-isomerization conditions. Simultaneously or sequentially, the 1-butene formed and/or the 1-butene present in the combined CC4 and dimerization product effluent may be isomerized into 2-butene at the SHU 305. Accordingly, operation 415 may include contacting 1-butene with the selective hydrogenation-isomerization catalyst to isomerize 1-butene into 2-butene under selective hydrogenation-isomerization conditions. The isomerization of 1-butene to 2-butene at the SHU 305 facilitates downstream separation of isobutylene from normal butylenes (for example, in separation unit 125). Suitable selective hydrogenation-isomerization conditions for operating the SHU 305 and selective hydrogenation-isomerization catalysts are described herein. The selective hydrogenation-isomerization product effluent may then be discharged from the SHU 305 through line L24.

The process 400 may further include separating isobutylene and 2-butene from the selective hydrogenation-isomerization product effluent at operation 420. Here, for example, the selective hydrogenation-isomerization product effluent comprising isobutylene and 2-butene may be fed through line L24 to the separation unit 125. At the separation unit 125, the selective hydrogenation-isomerization product effluent may be subjected to separation conditions effective to separate isobutylene and 2-butene from the selective hydrogenation-isomerization product effluent. The separation conditions may also be effective to separate the isobutylene from the 2-butene. The separation conditions may also be effective to separate 1-butene from isobutylene. Separation conditions and apparatus/techniques for separation at the separation unit 125 in operation 420 are described herein. The isobutylene (or isobutylene fraction) may then be discharged from the separation unit 125, for example, leaving “overhead” through line L29. The 2-butene (or 2-butene fraction) may also be discharged from the separation unit 125, for example, leaving as a heavier “bottoms” fraction through line L25.

Additionally, or alternatively, separation of isobutylene and 2-butene, and optionally 1-butene, from the selective hydrogenation-isomerization product effluent at operation 420 may involve: (i) an adsorptive separation (for example, molecular sieves or zeolites selective for olefin/paraffin separations); (ii) an extractive distillation using selective solvents (for example, solvents such as NMP, furfural, acetonitrile, or sulfolane); and/or (iii) a reactive conversion approach where 1-butene is shifted into a different C4 olefin form prior to separation (for example, reactive distillation to convert 1-butene to 2-butene utilizing a separation unit configured as a reactive distillation unit incorporating an acidic isomerization catalyst).

The process 400 may further include skeletal isomerizing at least a portion of the 2-butene separated to form a skeletal isomerization product effluent comprising isobutylene at operation 425. Here, at least a portion of the 2-butene fraction collected from the separation (at operation 420) may be sent to the skeletal isomerization reactor 115 to convert the 2-butene to isobutylene. This skeletal isomerization process at operation 425 increases the yield of isobutylene, useful for the downstream polymerization process. The skeletal isomerization process of operation 425 may include feeding the 2-butene separated from the selective hydrogenation-isomerization product effluent to a skeletal isomerization reactor 115 through line L25. The skeletal isomerization process of operation 425 may further include contacting the 2-butene with a skeletal isomerization catalyst to convert the 2-butene to isobutylene under skeletal isomerization conditions. Suitable skeletal isomerization conditions for operating the skeletal isomerization reactor 115 and skeletal isomerization catalysts are described herein. The skeletal isomerization product effluent may then be discharged from the skeletal isomerization reactor 115 through line L26. The skeletal isomerization product effluent includes isobutylene, and may optionally include 1-butene and/or 2-butene. As a result of the skeletal isomerization process at operation 425, the skeletal isomerization product effluent exiting the skeletal isomerization reactor 115 through line L26 may have a concentration of isobutylene that is higher than a concentration of isobutylene entering the skeletal isomerization reactor 115 through line L25.

The process 400 may further include recycling or feeding the skeletal isomerization product effluent to the SHU 305 at operation 430. The recycling or feeding process of operation 430 may include feeding the 2-butene through line L26 and L28 to the SHU 305. Additionally, or alternatively, the recycling or feeding process of operation 430 may include combining the 2-butene flowing through line L26 and the CC4 flowing through line L27 and then feeding this combined feed through line L28 to the SHU 305.

The process 400 may further include converting 1-butene present in the skeletal isomerization product effluent to 2-butene at operation 435. The conversion of 1-butene to 2-butene is performed in the SHU 305, under selective hydrogenation-isomerization conditions, and in the presence of a selective hydrogenation-isomerization catalyst. Operation 435 may be performed in the same or similar manner as described herein with respect to operation 415. Suitable selective hydrogenation-isomerization conditions for operating the SHU 305 and selective hydrogenation-isomerization catalysts are described herein. The selective hydrogenation-isomerization product effluent may then be discharged from the SHU 305 through line L24. This loop from SHU 305 to separation unit 125 to skeletal isomerization reactor 115 (e.g., operations 415-430) may facilitate high conversion of CC4 and ethylene dimer feeds to isobutylene.

Prior to dimerizing ethylene to produce the dimerization product effluent, process 400 may further optionally include converting light olefins into ethylene. Here, for example, light hydrocarbons (such as C2-C6 hydrocarbons, such as C2-C4 hydrocarbons) may be fed through line L21 into olefin-producing unit 105. The olefin-producing unit may include any suitable apparatus to form ethylene such as, for example, a steam cracker or a dehydrogenation reactor. At the olefin-producing unit 105, the light hydrocarbons may be converted under olefin-producing conditions effective to form ethylene. Olefin-producing conditions and the olefin-producing unit 105 are described herein. An olefin-containing effluent comprising the ethylene formed at olefin-producing unit 105 may then be discharged from the olefin-producing unit 105 through line L22. The olefin-containing effluent (also referred to as an ethylene stream) may then be fed to the ethylene dimerization reactor 110 where the ethylene is dimerized as described herein. Optionally, ethylene may be separated from the olefin-containing effluent and discharged from the olefin-producing unit 105 through line L22 prior to ethylene dimerization. This separation may be performed at the olefin-producing unit 105. Suitable separation techniques may include performing a distillation, a fractional distillation, a vacuum distillation, a flash evaporation, a fractionation, an extraction, a decantation, a coalescence, or combinations thereof, on the olefin-containing effluent.

CC4 may be present in the olefin-containing effluent. In some embodiments, which may be combined with other embodiments, the process 400 may optionally include separating CC4 from the olefin-containing effluent using, for example, one or more of those separation techniques described herein for separating ethylene from the olefin-containing effluent. The separation of CC4 from the olefin-containing effluent may be performed at the olefin-producing unit 105. This CC4 may then be discharged from olefin-producing unit 105 and then fed to the SHU 305 through lines L27 and L28 as described herein.

The process 400 may further optionally include an optional metathesis operation, whereby propylene is formed in the metathesis reactor 145 from 2-butene and ethylene. Here, at least a portion of the 2-butene separated from the selective hydrogenation-isomerization product effluent may be fed from separation unit 125 to the metathesis reactor 145 through line L25 and line L34. At least a portion of an ethylene stream produced at the olefin-producing unit 105 may be fed from the olefin-producing unit 105 to the metathesis reactor through line L22 and line L35. At the metathesis reactor 145, a mixture that includes a 2-butene (or 2-butene fraction) separated from the selective hydrogenation-isomerization product effluent and the ethylene stream may be formed by combining the two feeds. The mixture may contact the metathesis catalyst, under metathesis conditions, in the metathesis reactor 145 to form propylene. Suitable metathesis catalysts and metathesis conditions are described herein. Here, the metathesis reaction includes 2-butene reacting with ethylene to form two molecules of propylene. The propylene may be separated from the reaction product mixture to form a propylene stream. The resulting propylene stream may be discharged from the metathesis reactor 145 through line L36. This propylene stream is a valuable product stream. Use of the optional metathesis operation may further improve process integration and carbon efficiency of the system 300. Overall, the optional metathesis operation may provide a practical outlet for surplus 2-butene, improve overall carbon efficiency, and/or generate on-purpose propylene, aligning the process with modern petrochemical integration strategies and market demand.

The reaction product mixture comprising the desired propylene from the optional metathesis operation may further include unreacted 2-butene. The process 400 may further optionally include processing of the unreacted 2-butene (if any) present in the reaction product mixture after the optional metathesis operation. Such processing of the unreacted 2-butene may include recycling the unreacted 2-butene back to the metathesis reactor 145. Such recycling may facilitate the conversion efficiency of the metathesis. Additionally, or alternatively, processing of the unreacted 2-butene may include skeletal isomerizing the unreacted 2-butene to form isobutylene. Here, unreacted 2-butene from the optional metathesis operation may be flowed to the skeletal isomerization reactor 115 where it may be converted to isobutylene as described herein, for example, with respect to operation 425. Suitable skeletal isomerization conditions for operating the skeletal isomerization reactor 115 and skeletal isomerization catalysts are described herein.

The process 400 may further optionally include converting isobutylene oligomer coproducts into an olefin-containing effluent comprising ethylene. As described herein, isobutylene oligomer coproducts may be formed during isobutylene polymerization at the polymerization unit 130. The isobutylene oligomer coproducts may be converted into ethylene by, for example, (a) hydrotreating the isobutylene oligomer coproducts to form hydrotreated C8-C20 hydrocarbons; and (b) converting these hydrotreated C8-C20 hydrocarbons to ethylene. Here, isobutylene oligomer coproducts may be fed through line L37 to hydrotreatment reactor 135, where the isobutylene oligomer coproducts are converted, under hydrotreatment conditions and in the presence of a hydrotreatment catalyst, to the hydrotreated C8-C20 hydrocarbons. Suitable hydrotreatment conditions and hydrotreatment catalysts are described herein. The hydrocarbons may then be discharged from the hydrotreatment reactor 135 through line L38 and fed to olefin-producing unit 105. At the olefin-producing unit 105, the hydrocarbons may be converted to ethylene under olefin-producing conditions. Suitable olefin-producing conditions to form ethylene and olefin-producing catalysts are described herein. An olefin-containing effluent comprising the ethylene is produced from the conversion. The olefin-containing effluent may optionally include CC4. In such cases, ethylene and/or CC4 (in separate fractions) may be separated from the olefin-containing effluent by performing, for example, a distillation, a fractional distillation, a vacuum distillation, a flash evaporation, a fractionation, an extraction, a decantation, a coalescence, or combinations thereof, on the olefin-containing effluent. This separation of ethylene and/or CC4 from the olefin-containing effluent may be performed at olefin-producing unit 105. The ethylene, or olefin-containing effluent comprising ethylene, may be discharged from the olefin-producing unit 105 through line L22 or a separate line. The CC4 may be discharged from the olefin-producing unit 105 through line L27.

The process 400 may further optionally include combining dimerized ethylene effluent with the selective hydrogenation-isomerization product effluent for separation and isobutylene polymerization. This optional operation may include (a) combining the dimerization product effluent and the selective hydrogenation-isomerization product effluent; (b) separating isobutylene and 2-butene from the combined effluent (for example by fractional distillation), and feeding the isobutylene to polymerization unit 130 (e.g., leaving “overhead”) and feeding the heavier 2-butene to the skeletal isomerization reactor 115 (as described herein); (c) polymerizing the isobutylene into PIB at the polymerization unit 130 (as described herein); and (d) skeletal isomerizing the 2-butene into isobutylene at the skeletal isomerization reactor 115 (as described herein). (a) may be performed as described herein by adding the dimerization product effluent flowing in line L23 into the SHU 305 where it undergoes selective hydrogenation-isomerization. This provides for advantageous effects in the production of isobutylene including, for example, low cost and high yield potential.

Additionally, or alternatively, (a) may be performed by combining the dimerization product effluent flowing in line L23 with the selective hydrogenation-isomerization product effluent flowing through line L24 such that the dimerization product effluent bypasses the SHU 305. In connection with this optional operation of combining dimerized ethylene effluent with the selective hydrogenation-isomerization product effluent for separation and isobutylene polymerization, the separation unit 125 may be implemented as a reactive distillation unit incorporating an acidic isomerization catalyst. In this setup, at least a portion (or all) of leftover 1-butene present in the dimerization product effluent that bypasses the SHU 305 may be converted to 2-butene directly within the reactive distillation column. The resulting 2-butene will settle at the bottom of the column and may be sent to the skeletal isomerization reactor. Meanwhile, the isobutylene cut collected from the top of the reactive distillation unit is free, or significantly free (e.g., less than 1%), of 1-butene contamination. This reactive distillation “polishing” operation may be used as an alternative to treatment in the SHU or in addition to treatment in the SHU.

FIG. 4B shows selected operations of a process 450 for forming PIB according to at least one embodiment of the present disclosure. The process 450 may be performed using system 300. In some embodiments, which may be combined with other embodiments, process 450 provides an implementation of producing PIB, wherein ethylene dimerization product effluent and CC4 may be processed to enhance isobutylene production, including integration with selective hydrogenation-isomerization and recycling of 2-butene for skeletal isomerization. The isobutylene may then be separated and polymerized into PIB, such as HR-PIB.

The process 450 may include operation 405 (dimerization), operation 410 (feeding CC4 and dimerization product effluent to the SHU 305), operation 415 (selective hydrogenation-isomerization), operation 420 (separation), operation 425 (skeletal isomerization), operation 430 (recycle skeletal isomerization product effluent to the SHU 305), and/or operation 435 (converting 1-butene to 2-butene at the SHU 305) described herein with respect to process 400. The process 450 may optionally further include one or more of those optional operations described herein with respect to process 400. After operation 420, isobutylene (or an isobutylene fraction) flows through line L29.

The process 450 may further include polymerizing the isobutylene separated from the selective hydrogenation-isomerization product effluent into a polymerization product effluent at operation 455. Here, the isobutylene flowing in line L29 may be fed to a polymerization reactor of the polymerization unit 130. In the polymerization reactor, a polymerization reaction mixture that includes isobutylene and polymerization catalyst may be formed. The mixture may be reacted, under polymerization conditions, in the polymerization unit to convert the isobutylene to the polyisobutylene. During the polymerization, the isobutylene may contact the polymerization catalyst, under polymerization conditions, to convert the isobutylene to PIB, such as HR-PIB.

Suitable polymerization catalysts are described herein. As an illustrative, but non-limiting example, the polymerization catalyst may include BF₃. The polymerization catalyst may include a solid substrate (for example, silica-alumina) comprising sorbed BF₃ catalyst complex. The BF₃ catalyst complex may include BF₃ and a complexing agent comprising an oxygen-containing compound, such as an oxygen-containing compound that is free of beta-hydrogen atoms. The solid substrate comprising sorbed BF₃ catalyst complex (for example, BF₃·MeOH) may include any suitable amount of BF₃, for example, about 30 wt % or more of BF₃ based on the total wt % of the solid substrate comprising sorbed catalyst complex. Other suitable polymerization catalysts are contemplated.

Suitable polymerization conditions are described herein. As an illustrative, but non-limiting example, polymerization conditions may include a temperature in a range from about −10° C. to about 40° C.; a polymerization period/reaction time of about 4 minutes or less; or combinations thereof. Other suitable polymerization conditions are contemplated including those described herein. The polymerization reactor may include a tubular loop reactor. As described herein, tubular loop reactors facilitate high-velocity movement of the polymerization reaction mixture (the mixture comprising isobutylene and polymerization catalyst). The high-velocity reaction mixture may improve heat transfer/removal and catalyst dispersion. The heat transfer/removal and catalyst dispersion may help facilitate control (or precise control) of molecular weight distribution and/or may help facilitate high selectivity for terminal double bonds in the PIB product (that is, high selectivity for HR-PIB, such as HR-PIB having an alpha vinylidene content greater than 75 wt %). The controlled reaction conditions and advanced reactor design may help ensure a consistent product with a molecular weight distribution suitable for industrial applications such as fuel and lubricant additives, sealants, and adhesives, among other applications. Other suitable polymerization reactors, polymerization conditions, etc. are contemplated.

In some examples, this polymerization catalyst/polymerization reactor system may achieve greater than 80% alpha vinylidene content (for example, greater than 90% alpha vinylidene content) and narrow molecular weight distributions.

As described herein, the polymerization product effluent that includes the PIB, such as HR-PIB, may include one or more optional components such as isobutylene oligomer coproducts, unreacted isobutylene, hydrocarbon diluent, polymerization catalyst residues, or combinations thereof. The one or more optional components of the polymerization product effluent may be separated from the polymerization product effluent and/or may be recycled to various units in the system 300.

As an example, the process 450 may optionally include separating isobutylene oligomer coproducts from the polymerization product effluent by use of an oligomer separation unit in the polymerization unit 130. Here, the isobutylene oligomer coproducts may be separated by, for example, distillation as described herein whereby the isobutylene oligomer coproducts may be discharged from the polymerization unit 130 (e.g., leaving “overhead”) through line L30 and the PIB may be discharged from the polymerization unit 130 through line L33 (e.g., leaving as a bottoms stream).

The isobutylene oligomer coproducts flowing through line L30 may then be subjected to further processing, such as cracking, hydrotreatment, or combinations thereof. For example, the process 450 may further optionally include converting the isobutylene oligomer coproducts to isobutylene by cracking (e.g., thermal cracking). Here, the isobutylene oligomer coproducts may be fed to the cracking reactor 150 through line L30. At the cracking reactor 150, the isobutylene oligomer coproducts may contact a cracking catalyst, under cracking conditions, to convert the isobutylene oligomer coproducts to isobutylene. Suitable cracking conditions and cracking catalysts are described herein. The cracked isobutylene may be recycled back into the polymerization reactor of the polymerization unit 130. For example, the isobutylene may be discharged from the cracking reactor 150 through line L31 and may be fed to the isobutylene flowing through line L29 and into the polymerization unit 130, where the isobutylene may be polymerized in the polymerization reactor. Overall, the optional cracking operation on the isobutylene oligomer coproducts and subsequent polymerization of the isobutylene may help minimize waste, may help enhance feedstock utilization, and may address potential (if any) selectivity losses from the oligomer formation by converting the isobutylene oligomer coproducts into useful intermediates.

Additionally, or alternatively, the process 450 may further optionally include hydrotreating the isobutylene oligomer coproducts in hydrotreatment reactor 135. The isobutylene oligomer coproducts may be fed to the hydrotreatment reactor 135 through line L30 and L37. The hydrotreatment converts the isobutylene oligomer coproducts to hydrotreated C8-C20 hydrocarbons. As a recycled feedstock, the hydrotreated C8-C20 hydrocarbons (exiting the hydrotreatment reactor 135 through line L38) may be processed in an olefin-producing unit 105 (for example, a steam cracker) to produce additional ethylene, CC4, and/or other valuable products, closing the loop on waste and improving the overall efficiency of the process. For example, at the olefin-producing unit 105, hydrocarbons fed from the hydrotreatment reactor 135 and into the olefin-producing unit 105 may be converted to ethylene, propylene, or combinations thereof. This optional hydrotreatment and conversion to ethylene is described herein with respect to process 200.

The ability to subject the isobutylene oligomer coproducts flowing through line L30 to further processing, such as cracking, hydrotreatment, or combinations thereof may facilitate a sustainable, low-waste design in which all carbon (or substantially all) either enters PIB production or is returned to the front end of the process for reuse.

The process 450 may further optionally include hydrogenating the PIB (for example, HR-PIB) flowing through line L33. Here, the PIB flowing through line L33 may be fed to a hydrogenation reactor (not shown in FIG. 3). At the hydrogenation reactor, H₂ gas may contact a mixture comprising PIB and a hydrogenation catalyst, under hydrogenation conditions, to convert the PIB into the hydrogenated PIB (such as hydrogenated HR-PIB). Suitable hydrogenation catalysts and hydrogenation conditions for converting PIB to hydrogenated PIB are described herein. The hydrogenated PIB (for example, hydrogenated HR-PIB) is described herein,

In some implementations, which may be combined with other implementations, embodiments described herein may include one or more of the following. A light hydrocarbon stream, such as a C2-C4 hydrocarbon stream such as ethane, propane, butane, or combinations thereof, such as ethane, may be fed into a steam cracker (or other olefin-producing unit 105), where ethylene is produced as a primary output, along with a CC4 side stream as a minor coproduct. When ethane is cracked, the CC4 may include 1,3-butadiene, 1-butene, 2-butene, or combinations thereof, and optionally smaller amounts of isobutylene. The CC4 stream may be directed to the SHU 305 with isomerization capability, where 1,3-butadiene may first be converted to 1-butene and then to 2-butene, along with pre-existing 1-butene. Isobutylene may remain unreacted. The selective hydrogenation-isomerization effluent, comprising isobutylene and 2-butene, may be fed into separation unit 125 (e.g., a fractional distillation column), where isobutylene may be separated overhead and may then be polymerized at polymerization unit 130 to PIB, such as HR-PIB, and optionally a small quantity of isobutylene oligomer coproduct. The 2-butene from the separation unit 125 (e.g., a fractional distillation column) may be recycled back to the SHU 305 through skeletal isomerization reactor 115. Meanwhile, the ethylene from the steam cracker (or other olefin-producing unit 105) may be fed into ethylene dimerization reactor 110, producing a mixture of C4 olefins, primarily 1-butene and 2-butene, with a minor amount of isobutylene. This mixed C4 stream may be combined with the CC4 stream and processed in the SHU 305, ultimately yielding isobutylene. The isobutylene oligomer coproducts from the PIB polymerization process may be cracked to produce isobutylene in an oligomer cracking unit (e.g., cracking reactor 150) and may be recycled back into the isobutylene feed for the PIB polymerization process at polymerization unit 130.

With respect to systems described herein, for example, system 100 and system 300, the lines may be coupled to inlets and outlets of the reactors, units, or other lines to allow various streams or feeds to flow through the various reactors, units, and lines of the system 100 and the system 300. For example, in system 100, the ethylene dimerization reactor 110 may include a first outlet coupled to line L3 and the skeletal isomerization reactor 115 may have a first inlet coupled to line L3. This allows the ethylene dimerization product effluent to exit the ethylene dimerization reactor 110 through the first outlet of the ethylene dimerization reactor 110, flow to the skeletal dimerization reactor through line L3, and enter the skeletal isomerization reactor 115 through the inlet of the skeletal isomerization reactor 115.

Although not shown in FIG. 1 and FIG. 3, it should be understood that suitable equipment for controlling, for example, temperature, pressure, and flow control of various feeds, effluents, and output streams may be used with the system 100 or the system 300. For example, heat exchangers may be used to cool or heat a liquid or a gas along one or more lines or within various units or reactors of the system 100 or the system 300. Pumps and motors may be utilized to control the rate of flow of the materials traveling or flowing through the lines and the operating pressures of various components of the system 100 or the system 300. Further, the system 100 or the system 300 may include valves or other release mechanisms for, e.g., purging gases or liquids from the system. Various process controls may be used. Such process controls may include probes and sensors such as pressure indicators, differential pressure cells, temperature indicators, thermocouples, temperature switches, resistance temperature detectors, solenoids, flowmeters, flow regulators and valves, gas analyzers, humidity sensors, radar sensors, ammeters, current meters, liquid level detectors, feed level probes, electrical drives, and combinations thereof.

Optionally, and as described herein, one or more elements described with respect to the system 100 and the system 300 may be coupled to a controller, for example, controller 160. Although not shown, the controller 160 may be coupled to the various elements (e.g., units, reactors, lines, etc.) of system 100 and system 300 by use of a suitable line or appropriate wiring. The controller 160 may be utilized to control, for example, one or more operating parameters of the one or more elements illustrated in the system 100 and the system 300, one or more operations of processes described herein (for example, one or more operations of process 200, process 250, process 400, process 450), or combinations thereof. The controller 160 may include a processor, memory, and support circuits. The processor may be one of any form of general purpose microprocessor, or a general purpose central processing unit (CPU), each of which may be used in an industrial setting, such as a programmable logic controller (PLC), supervisory control and data acquisition (SCADA) systems, or other suitable industrial controller.

The memory of the controller 160 is non-transitory and may be one or more of readily available memory such as random access memory (RAM), read only memory (ROM), or any other form of digital storage, local or remote. The memory contains instructions, that when executed by the processor, may facilitate the operation of one or more elements illustrated in system 100 or system 300, one or more operations of process 200, one or more operations of process 250, one or more operations of process 400, one or more operations of process 450, or combinations thereof. The instructions in the memory are in the form of a program product such as a program that implements the method of the present disclosure. The program code of the program product may conform to any one of a number of different programming languages. Illustrative computer-readable storage media include, but are not limited to: (i) non-writable storage media (for example, read-only memory devices within a computer such as CD-ROM disks readable by a CD-ROM drive, flash memory, ROM chips, or any type of solid-state non-volatile semiconductor memory) on which information is permanently stored; and (ii) writable storage media (for example, floppy disks within a diskette drive or hard-disk drive or any type of solid-state random-access semiconductor memory) on which alterable information is stored. Such computer-readable storage media, when carrying computer-readable instructions that direct the functions of the methods described herein, are examples of the present disclosure. The disclosure may be, for example, implemented as the program product stored on a computer-readable storage media (for example, memory) for use with a computer system (not shown). The program(s) of the program product define functions of the disclosure, described herein.

As an example with system 100, the controller 160 may be coupled to the olefin-producing unit 105, the ethylene dimerization reactor 110, the skeletal isomerization reactor 115, the isomerization reactor 120, the separation unit 125, the line L6, or combinations thereof. The controller 160 may be configured to cause the ethylene dimerization reactor: to receive ethylene from the olefin-producing unit; to convert the ethylene to a dimerization product effluent comprising normal butylene; to discharge the dimerization product effluent; or combinations thereof.

The following examples are put forth so as to provide those of ordinary skill in the art with a complete disclosure and description of how to make and use embodiments of the present disclosure, and are not intended to limit the scope of embodiments of the present disclosure. Efforts have been made to ensure accuracy with respect to numbers used (e.g., amounts, dimensions, etc.) but some experimental errors and deviations should be accounted for.

### EXAMPLES

### Introduction

Examples of forming isobutylene and polymer compositions (e.g., HR-PIB) were performed by embodiments of the present disclosure. The Test Methods describes, e.g., methods of characterizing isobutylene produced by embodiments described herein and polymer compositions produced by embodiments described herein.

### Test Methods

Kinematic viscosity at 100° C. (KV100) was determined according to ASTM D445.

Polymer Compositions. The type and amount of each olefin isomer (e.g., alpha vinylidene, beta vinylidene, and other isomers) was determined by ¹³C NMR. ¹³C NMR spectra were collected using a 500 MHz Bruker pulsed Fourier transform NMR spectrometer equipped with a 10 mm Broad Band Observation (BBO) probe at about room temperature. The polymer sample was dissolved in chloroform-d (CDCl₃) and transferred into a 10 mm glass NMR tube. Typical acquisition parameters were inverse-gated (IG) decoupling, a 90° pulse, and a 40 second relaxation delay. Chemical shifts were determined relative to the CDCl₃ signal which is set to about 77.2 ppm. To achieve maximum signal-to-noise for quantitative analysis, multiple data files may be added together. The spectral width was adjusted to include all of the NMR resonances of interest. ¹³C NMR chemical shifts (CDCl₃) for the olefin carbon atoms are provided below in Table 1. All data provided in Table 1 is approximate values. In Table 1, the chemical shift provided corresponds to the carbon underlined.

TABLE 1
¹³C NMR Chemical Shifts of Polymer Compositions

| Type of Olefin Isomer | Chemical Shifts, ppm |
|---|---|
| alpha vinylidene isomer | 143 (RC(CH₃)═CH₂); |
| | 115 (RC(CH₃)═CH₂) |
| beta vinylidene isomer | 136 (RC(H)═C(CH₃)₂); |
| | 128 (RC(H)═C(CH₃)₂) |
| terminal trisubstituted | 134 (RC(CH₃)═CH(CH₃)); |
| vinylidene isomer (1) | 123 (RC(CH₃)═CH(CH₃)) |
| terminal trisubstituted | 139 (RC(H)═C(CH₃)(CH₂CH₃)); |
| vinylidene isomer (2) | 130 (RC(H)═C(CH₃)(CH₂CH₃) |
| terminal tetrasubstituted | 133 (RC(CH₃)═C(CH₃)₂); |
| vinylidene isomer | 122 (RC(CH₃)═C(CH₃)₂) |
| internal disubstituted | 149 (RC(═CH₂)(CH₃)); |
| vinylidene isomer | 111 (RC(═CH₂)(CH₃)) |

When the alpha vinylidene isomer content (wt %) was determined to be about 75 wt % or greater, the polymer composition comprises HR-PIB. When the alpha vinylidene isomer content (wt %) was determined to be less than 75 wt %, the polymer composition comprises a mid-range vinylidene PIB. Generally, HR-PIB has the following content: alpha vinylidene content (≥75 wt % or more); beta vinylidene isomer (<10-15 wt %); terminal trisubstituted vinylidene isomer (1) (<1 wt %); terminal trisubstituted vinylidene isomer (2) (<2-5 wt %); terminal tetrasubstituted vinylidene isomer (<2-5 wt %); internal disubstituted vinylidene isomer (<2-5 wt %).

Polymer molecular weight. Molecular weights (weight-average molecular weight, Mw, and number-average molecular weight, Mn), and PDI (Mw/Mn) were determined using gel permeation chromatography (GPC). Equipment included a Waters Alliance 2695 HPLC system with a differential refractive index detector (DRI). A typical GPC procedure was to dissolve the sample to be tested in tetrahydrofuran (THF) at a concentration of about 1 wt % to about 10 wt %. The polymer solution was pumped through a series of columns packed with Styragel beads of known porosity. Typical pore diameters range from about 10,000 Å down to about 50-100 Å, and a typical column string includes a 10⁴ Å column, a 10³ Å column, a 1000 Å column and a 2-100 Å column. For example, Waters Styragel HR columns 1, 3, and 4 may be used. The nominal flow rate was about 1.0 mL/min. The various transfer lines, columns, and differential refractometer (the DRI detector) were contained in an oven maintained at about 40° C. Elution solvent was THF. There was a 105-sample carousel for automatic injections. Empower 2 was the software system for controlling the separation and analysis.

The columns were calibrated with known molecular weight standards, both narrow distribution standards and broad distribution standards (for example, polystyrene standards from a molecular weight of 500 to 400K). From the calibration, Mn and Mw were determined for a polymer sample.

Polymer solutions for GPC were prepared by placing the dry polymer in a glass container, adding the desired amount of THF, and then filtering the mixture through a 0.45-micron nylon or polytetrafluoroethylene (PTFE) filter. All quantities were measured gravimetrically. The concentration of polymer to THF was about 10 mg/mL to 20 mg/mL.

Prior to running each sample, the DRI detector and the injector were purged. Flow rate in the apparatus was then increased to about 0.5 mL/minute, and the DRI was allowed to stabilize for about 8 hours to about 9 hours before injecting the first sample. Each sample run takes about one hour to complete.

Isobutylene conversion to PIB and percent yield. Isobutylene conversion to HR-PIB was determined by gas chromatography according to ASTM D424-09 on an Agilent 6890 Gas Chromatograph with dual flame ionization detector using a 30 meter Restek RTX column and Zero grade nitrogen carrier gas at a flow rate of about 30 cc/min with a split ratio of 10. Percent yield of HR-PIB was determined gravimetrically by dividing the weight of the HR-PIB product recovered by the weight of the isobutylene used in the polymerization and multiplying by 100. Selectivity was determined gravimetrically by dividing the weight of the HR-PIB product recovered by the sum of the oligomers and HR-PIB produced and multiplying by 100.

Table 2 shows illustrative, but non-limiting, feeds/streams flowing through selected lines of the system 100.

TABLE 2

| Line | Feed/stream |
|---|---|
| L1 | Light hydrocarbons |
| L2 | Ethylene |
| L3 | 1-butene and/or 2-butene |
| L4 | Isobutylene, with 1-butene and/or 2-butene |
| L5 | Isobutylene with 2-butene |
| L6 | 2-butene |
| L7 | Isobutylene |
| L8 | PIB |
| L9 | Isobutylene oligomer coproducts |

Table 3 shows illustrative, but non-limiting, feeds/streams flowing through selected lines of the system 300.

TABLE 3

| Line | Feed/stream |
|---|---|
| L21 | Light hydrocarbons |
| L22 | Ethylene |
| L23 | 1-butene and/or 2-butene |
| L24 | Isobutylene with 2-butene |
| L25 | 2-butene |
| L26 | Isobutylene, with 1-butene and/or 2-butene |
| L27 | CC4 |
| L29 | Isobutylene |
| L30 | Isobutylene oligomer coproducts |
| L33 | PIB |

### Example 1: Conversion of Ethylene to Isobutylene

A continuous feed of 35 gallons per minute (gpm) of liquid ethylene, corresponding to approximately 90 kilograms per minute (198 pounds per minute), was introduced into an ethylene dimerization reactor. The ethylene dimerization reactor was operated at a temperature of about 60° C., a pressure of about 3 MPa, and a liquid hourly space velocity of about 5 h⁻¹ in the presence of a nickel complex catalyst. Under these conditions, ethylene conversion was approximately 95%. The dimerization product effluent included about 85.5 kg/min of converted ethylene, of which approximately 98% was recovered as C4 butylenes and about 2% as higher oligomers, with about 4.5 kg/min of unreacted ethylene present. Thus, the effluent stream discharged from the dimerization step contained approximately 83.8 kg/min of normal butylenes (a mixture of 1-butene and 2-butene), together with about 1.7 kg/min of oligomeric C6+ material.

The dimerization product effluent was next introduced into a skeletal isomerization reactor containing a zeolitic catalyst (H-mordenite). The skeletal isomerization reactor was operated at about 400° C. and a pressure of about 0.2 MPa. Under these conditions, the butylenes were redistributed such that the skeletal isomerization product effluent included approximately 40 wt % isobutylene (33.5 kg/min), 30 wt % 2-butene (25.1 kg/min), and 30 wt % 1-butene (25.1 kg/min), with the oligomeric fraction passing through largely unchanged.

The skeletal isomerization effluent was then directed to a butene isomerization reactor operated at about 150° C. and 1 MPa in the presence of platinum on alumina (Pt/Al₂O₃) catalyst. In this operation, approximately 90% of the 1-butene present was converted to 2-butene. As a result, the isomerization effluent contained about 2.5 kg/min of residual 1-butene and about 22.6 kg/min of newly formed 2-butene, which combined with the original 25.1 kg/min of 2-butene, provided a total of about 47.8 kg/min of 2-butene. The isobutylene fraction of 33.5 kg/min remained essentially unchanged through this step. The final mixed C4 effluent, therefore, included approximately 33.5 kg/min isobutylene, 47.8 kg/min 2-butene, and 2.5 kg/min 1-butene, in addition to minor oligomers and traces of unconverted ethylene.

This mixed C4 effluent was passed to a separation unit, where fractional distillation was employed to recover a high purity isobutylene stream. The separation yielded an isobutylene product stream of about 33.5 kg/min (≥99 wt % purity) and a raffinate stream of about 50.3 kg/min, comprising predominantly 2-butene with a minor residual amount of 1-butene. The yield of isobutylene based on ethylene was about 37.2%.

### Example 2: Conversion of Isobutylene from Example 1 to HR-PIB

The isobutylene product stream obtained in Example 1, comprising approximately 33.5 kg/min (2010 kg/h) of high purity isobutylene, was directed to a polymerization reactor for conversion to HR-PIB. The polymerization The polymerization reactor was operated at about 65° C. with an average residence time of about 185 seconds.

Polymerization was conducted in the presence of a solid BF₃·MeOH complex catalyst as a polymerization catalyst. The polymerization catalyst was charged at a concentration of 2,000 ppm based on isobutylene feed, corresponding to approximately 4.0 kg/h of polymerization catalyst relative to the 2,010 kg/h isobutylene throughput. No cosolvent was employed. Following polymerization, the polymerization product effluent was filtered to remove catalyst solids, yielding a clarified hydrocarbon mixture.

The polymerization product effluent was first directed to a debutanizer column, where unreacted isobutylene was condensed overhead and recycled. The HR-PIB and oligomer fractions were removed from the bottoms and further purified in a vacuum stripping column operated at 240° C. and 100 mm Hg (13 kPa), where isobutylene oligomer coproducts were removed overhead. The purified HR-PIB was withdrawn as the final product stream.

Under these conditions, approximately 85% of isobutylene was converted, and of the converted fraction, about 93% was selectively formed into HR-PIB with the balance being oligomers. This resulted in a production rate of about 26.5 kg/min HR-PIB (1590 kg/h) and about 1.6 kg/min oligomers (96 kg/h), with about 5.0 kg/min unreacted isobutylene (300 kg/h) discharged for recycle. The resulting HR-PIB had a Mn of about 1005 g/mole, a KV100 of about 221 cSt, and an alpha vinylidene content of about 83.6%.

### Example 3: Conversion of Ethylene+CC4 to Isobutylene

This example demonstrates a SHU-Fractionation-SKIP recycle loop. An integrated embodiment was operated in which an ethylene stream and a crude C4 (CC4) stream were combined and processed through dimerization, selective hydrogenation-isomerization, fractionation, and skeletal isomerization to yield isobutylene as the principal product.

An ethylene feed was introduced to the dimerization reactor at a rate of 15 gallons per minute (38.6 kg/min). Under typical dimerization conditions (60° C., 3 MPa, nickel complex catalyst), approximately 95% of the ethylene was converted, with about 98% selectivity to C4 olefins. The dimerization product effluent therefore contained approximately 36 kg/min of normal butylenes (primarily 1-butene and 2-butene), along with minor oligomers.

In parallel, a CC4 stream was supplied at a rate of 15 gallons per minute (38.6 kg/min). The CC4 composition, by weight, included about 69% butadiene, 10% 1-butene, 5.4% 2-butene, 13.6% n-butane, and small fractions of C1-C3 hydrocarbons and C5 hydrocarbons, with no isobutylene or isobutane present. This CC4 feed was combined with the dimerization product effluent and introduced into the SHU for selective hydrogenation-isomerization. In this operation, the butadiene was selectively hydrogenated to 2-butene, and a major portion of the 1-butene was isomerized to 2-butene. The selective hydrogenation-isomerization product effluent was enriched in 2-butene and contained minor amounts of residual 1-butene, together with inert components such as n-butane, C1-C3 hydrocarbons, and C5+ hydrocarbons.

The selective hydrogenation-isomerization product effluent was then separated by fractionated to recover a 2-butene-rich stream, while inerts were removed by controlled purge.

The 2-butene-rich stream was introduced into a skeletal isomerization reactor containing an acidic zeolite catalyst. In this reactor, 2-butene was rearranged to isobutylene, and the skeletal isomerization product effluent was recycled to the SHU. With recycle, the loop approached steady state, where unconverted 1-butene was further isomerized to 2-butene and trace dienes were fully hydrogenated.

Under continuous operation, the combined feed of 15 gpm ethylene and 15 gpm CC4 (77 kg/min total) provided approximately 68.5 kg/min of C4 olefins available for conversion. In practice, a portion of these olefins was lost through normal side reactions and purge requirements. About 4% of the olefins were over-hydrogenated to n-butane in the SHU, 3% were converted to C1-C3 light gases, 3% were diverted to C5+ heavy ends in the skeletal isomerization reactor, and an additional 3% of 2-butene was lost in the purge stream. These combined factors correspond to approximately 13% of the C4 olefin pool.

After accounting for these losses, the net isobutylene production rate was approximately 59.6 kg/min, recovered and an overhead stream in the fraction unit corresponding to a yield of about 87 wt % based on the C4 olefins present in the combined feeds, or about 77 wt % relative to the total feed streams.

This example demonstrates that even under practical operating conditions, with typical process losses due to over-hydrogenation, light-end and heavy-end formation, and purge requirements, the SHU-Fractionation-SKIP recycle loop (as described herein with respect to, e.g., FIG. 3 and FIG. 4A) efficiently converts the majority of ethylene-derived and CC4-derived olefins, including butadiene, into isobutylene as the principal product.

### Example 4: Conversion of Isobutylene from Example 3 to HR-PIB

The isobutylene product stream obtained in Example 3, comprising approximately 59.6 kg/min of high purity isobutylene, was introduced into a continuous loop polymerization reactor for conversion to HR-PIB. The polymerization reactor was operated at a temperature of about 25° C. with an average residence time of approximately 225 seconds.

Polymerization was conducted in the presence of a solid BF₃·MeOH complex catalyst charged at a concentration of 3,000 ppm based on isobutylene feed, corresponding to approximately 10.8 kg/h of polymerization catalyst relative to the 3,576 kg/h isobutylene throughput. No cosolvent was employed. Following polymerization, the polymerization product effluent was passed through a filtration unit to remove entrained catalyst solids.

Under these conditions, isobutylene conversion was approximately 67%, with a selectivity of about 97% to HR-PIB and the remainder forming isobutylene oligomer coproducts. Based on the isobutylene feed of 59.6 kg/min (˜3576 kg/h), the polymerization yielded approximately 2,324 kg/h of HR-PIB and about 71.8 kg/h of oligomers, with about 1,180 kg/h of unreacted isobutylene discharged from the polymerization reactor. The polymerization product effluent was directed first to a debutanizer column operated at a pressure sufficient to condense the unreacted isobutylene overhead. This fraction was cooled, collected, and recycled for further polymerization. The HR-PIB and oligomer fraction were removed from the column bottoms and subjected to vacuum stripping at about 240° C. and 100 mm Hg, removing light oligomers overhead. The purified HR-PIB product was withdrawn from the base of the stripper. The purified HR-PIB had an Mn of 2,325, a KV100 of about 1,665 cSt, and an alpha vinylidene content of about 87.1%.

### EMBODIMENTS LISTING

The present disclosure provides, among others, the following embodiments, each of which may be considered as optionally including any alternate embodiments:

Embodiment A1. A process for producing isobutylene, the process comprising:
dimerizing ethylene to produce a dimerization product effluent comprising a normal butylene;
skeletal isomerizing at least a portion of the normal butylene present in the dimerization product effluent to form a skeletal isomerization product effluent comprising isobutylene;
isomerizing at least a portion of 1-butene present in the skeletal isomerization product effluent to form an isomerization product effluent comprising 2-butene; and
optionally separating isobutylene and 2-butene from the isomerization product effluent.

Embodiment A2. The process according to Embodiment A1, wherein dimerizing ethylene to produce the dimerization product effluent comprises one or more of:
feeding the ethylene to an ethylene dimerization reactor;
contacting, under dimerization conditions, the ethylene with a dimerization catalyst (for example, a nickel-based catalyst) in the ethylene dimerization reactor to convert the ethylene to the normal butylene;
operating the ethylene dimerization reactor under the dimerization conditions, the dimerization conditions optionally comprising: a temperature in a range from about 50 to about 150° C., such as from about 75 to about 125° C., such as from about 95 to about 105° C.; a pressure in a range from about 1 to about 3 MPa, such as from about 1.5 to about 2.5 MPa, such as from about 1.75 to about 2.25 MPa; or a combination thereof; and/or
discharging the dimerization product effluent comprising the normal butylene.

Embodiment A3. The process according to any one of Embodiments A1-A2, wherein skeletal isomerizing at least a portion of the normal butylene comprises one or more of:
feeding the dimerization product effluent to a skeletal isomerization reactor, the dimerization product effluent comprising 1-butene, 2-butene, or combinations thereof;
contacting, under skeletal isomerization conditions, the 1-butene, 2-butene, or both with an acidic catalyst (for example, silica-alumina) in the skeletal isomerization reactor to convert the 1-butene, 2-butene, or both to isobutylene;
operating the skeletal isomerization reactor under the skeletal isomerization conditions, the skeletal isomerization conditions optionally comprising: a temperature in a range from about 200 to about 500° C., such as from about 200 to about 400° C., such as from about 250 to about 350° C., such as from about 275 to about 325° C., or from about 450 to about 500° C.; a pressure of about atmospheric pressure; or a combination thereof; and/or
discharging the skeletal isomerization product effluent comprising isobutylene, optionally 1-butene, and optionally 2-butene.

Embodiment A4. The process according to any one of Embodiments A1-A3, wherein isomerizing at least a portion of the 1-butene present in the skeletal isomerization product effluent into 2-butene comprises one or more of:
feeding the skeletal isomerization product effluent to an isomerization reactor;
contacting, under isomerization conditions, 1-butene present in the skeletal isomerization product effluent with an isomerization catalyst (for example, an acidic catalyst) in the isomerization reactor to convert 1-butene to 2-butene;
the isomerization reactor comprises a fixed-bed reactor;
operating the isomerization reactor under the isomerization conditions, the isomerization conditions optionally comprising: a temperature in a range from about 100 to about 200° C., such as from about 125 to about 175° C., such as from about 140 to about 160° C.; a pressure in a range from about 0.5 to about 1.5 MPa, such as from about 0.75 to about 1.25 MPa, such as from about 0.9 to about 1.1 MPa; or a combination thereof; and/or
discharging the isomerization product effluent comprising isobutylene and 2-butene.

Embodiment A5. The process according to any one of Embodiments A1-A4, wherein optionally separating the isobutylene and the 2-butene from the isomerization product effluent comprises one or more of:
feeding the isomerization product effluent to a separation unit;
operating the separation unit under separation conditions effective to separate the isobutylene and the 2-butene from the isomerization product effluent;
operating the separation unit under separation conditions effective to separate the isobutylene from the 2-butene;
discharging the 2-butene from the separation unit; and/or
discharging the isobutylene from the separation unit.

Embodiment A6. The process according to Embodiment A5, wherein, after separating the isobutylene and the 2-butene from the isomerization product effluent, the process further comprises: skeletal isomerizing the 2-butene separated from the isomerization product effluent to convert the 2-butene separated to isobutylene.

Embodiment A7. The process according to any one of Embodiments A1-A6, wherein the process further comprises:
converting light hydrocarbons (for example, C2-C6 hydrocarbons) into a first olefin-containing effluent comprising ethylene, wherein optionally at least a portion of the ethylene dimerized to produce the dimerization product effluent is sourced from the first olefin-containing effluent; and/or
(b) converting isobutylene oligomer coproducts into a second olefin-containing effluent comprising ethylene, wherein optionally at least a portion of the ethylene dimerized to produce the dimerization product effluent is sourced from the second olefin-containing effluent.

Embodiment A8. The process according to Embodiment A7, wherein (a) converting the light hydrocarbons comprises one or more of:
feeding the light hydrocarbons to an olefin-producing unit;
the converting the light hydrocarbons is performed in the olefin-producing unit under olefin-producing conditions to form ethylene;
and/or discharging ethylene from the olefin-producing unit and feeding the ethylene to an ethylene dimerization reactor.

Embodiment A9. The process according to any one of Embodiments A7-A8, wherein (b) converting the isobutylene oligomer coproducts comprises one or more of:
feeding the isobutylene oligomer coproducts to a hydrotreatment reactor;
contacting, under hydrotreatment conditions, the isobutylene oligomer coproducts with a hydrotreatment catalyst and hydrogen in the hydrotreatment reactor to convert the isobutylene oligomer coproducts to hydrotreated C8-C20 hydrocarbons in the hydrotreatment reactor (wherein the hydrotreatment conditions optionally comprise: operating the hydrotreatment reactor at a temperature in a range from about 200 to about 350° C.; and/or operating the hydrotreatment reactor at a pressure in a range from about 0.5 to about 10 MPa);
discharging the hydrotreated C8-C20 hydrocarbons;
feeding the hydrotreated C8-C20 hydrocarbons to an olefin-producing unit (for example, the olefin-producing unit being the same or different from the olefin-producing unit of Embodiment A8);
converting the hydrotreated C8-C20 hydrocarbons to the second olefin-containing effluent comprising ethylene in the olefin-producing unit;
optionally separating the ethylene from the second olefin-containing effluent; and/or
discharging the ethylene separated.

Embodiment A10. The process according to any one of Embodiments A7-A9, wherein the isobutylene oligomer coproducts are formed by: polymerizing the isobutylene separated from the isomerization product effluent into a polymerization product effluent, the polymerization product effluent comprising the isobutylene oligomer coproducts.

Embodiment A11. The process according to Embodiment A10, wherein:
the polymerization product effluent further comprises PIB; and
the process further comprises hydrogenating the PIB to form hydrogenated PIB, the hydrogenated PIB comprising a partially unsaturated PIB, a fully saturated PIB, or combinations thereof.

Embodiment A12. The process according to any one of Embodiments A1-A10, wherein the process further comprises:
forming a mixture comprising at least a portion of the 2-butene separated from the isomerization product effluent, and an ethylene stream (the ethylene stream optionally sourced from an olefin-producing unit that is the same or different from the olefin-producing unit of Embodiment A8); and
contacting the mixture with a metathesis catalyst, under metathesis conditions, in a metathesis reactor to form a metathesis product effluent comprising propylene.

Embodiment A13. The process according to Embodiment A12, wherein: the metathesis catalyst comprises a metal oxide of W, Mo, or Re, or a molybdenum alkylidene catalyst, or a rhenium alkylidene catalyst, or combinations thereof; the metathesis conditions comprise a temperature in a range from about 200 to about 350° C., such as from about 250 to about 300° C.; and/or the metathesis conditions comprise a pressure in a range from about 2 to about 5 MPa, such as from about 3 to about 4 MPa.

Embodiment A14. The process according to any one of Embodiments A1-A13, wherein the process further comprises:
(a) removing unreacted 2-butene from the metathesis product effluent; and
(b) one or more of (b1) or (b2):
   (b1) recycling unreacted 2-butene removed from the metathesis product effluent back to the metathesis reactor; and/or
   (b2) skeletal isomerizing the unreacted 2-butene separated from the metathesis product effluent to form isobutylene.

Embodiment A15. The process according to any one of Embodiments A1-A14, wherein the isomerization of 1-butene to 2-butene during isomerizing at least a portion of 1-butene present in the skeletal isomerization product effluent to form an isomerization product effluent comprising 2-butene is performed to facilitate separation of the isobutylene from linear butylenes present in the isomerization product effluent.

Embodiment A16. The process according to any one of Embodiments A1-A15, wherein the ethylene dimerized to produce a dimerization product effluent comprising the normal butylene is sourced from:
the C2-C6 hydrocarbons converted to the ethylene in the olefin-producing unit;
the isobutylene oligomer coproducts converted to the ethylene in the olefin-producing unit; or
a combination thereof.

Embodiment A17. The process according to any one of Embodiments A1-A16, wherein the process further comprises:
feeding a 1-butene-containing stream (for example, the dimerization product effluent) to a single combination reactor;
isomerizing 1-butene present in the 1-butene-containing stream to 2-butene in the single combination reactor;
skeletally isomerizing the 2-butene to isobutylene in the single combination reactor; and/or
discharging, from the single combination reactor, an effluent comprising isobutylene and 2-butene and that is substantially free of 1-butene.

Embodiment A18. The process according to Embodiment A17, wherein the 1-butene-containing stream comprises at least a portion of the dimerization product effluent.

Embodiment B1. A process for producing PIB (for example, HR-PIB), the process comprising:
(a) a process for producing isobutylene, comprising:
   dimerizing ethylene to produce a dimerization product effluent comprising a normal butylene;
   skeletal isomerizing at least a portion of the normal butylene present in the dimerization product effluent to form a skeletal isomerization product effluent comprising isobutylene;
   isomerizing at least a portion of 1-butene present in the skeletal isomerization product effluent to form an isomerization product effluent comprising 2-butene; and
   separating isobutylene and 2-butene from the isomerization product effluent; and
(b) polymerizing the isobutylene separated from the isomerization product effluent into a polymerization product effluent comprising PIB (for example, HR-PIB).

Embodiment B2. The process according to Embodiment B1, wherein (a) the process for producing isobutylene comprises any one of Embodiments A1-A18.

Embodiment B3. The process according to any one of Embodiments B1-B2, wherein polymerizing the isobutylene comprises one or more of:
feeding the isobutylene to a polymerization unit;
forming a polymerization reaction mixture comprising the isobutylene and a polymerization catalyst;
reacting the polymerization reaction mixture, under polymerization conditions, in the polymerization unit to convert the isobutylene to the PIB; and/or
discharging the polymerization product effluent comprising the PIB.

Embodiment B4. The process according to Embodiment B3, wherein the polymerization conditions comprise: a temperature in a range from about −10° C. to about 40° C.; and/or a polymerization period of about 4 minutes or less.

Embodiment B5. The process according to any one of Embodiments B3-B4, wherein the polymerization catalyst comprises BF₃ (for example, a BF₃ catalyst complex, for example, a solid BF₃ catalyst complex).

Embodiment B6. The process according to any one of Embodiments B3-B5, wherein the polymerization catalyst comprises a BF₃ catalyst complex sorbed on a solid substrate (for example, silica-alumina), the BF₃ catalyst complex comprising: BF₃; and a complexing agent comprising an oxygen-containing compound, the oxygen-containing compound is free of beta-hydrogen atoms.

Embodiment B7. The process according to Embodiment B6, wherein the solid substrate comprising sorbed BF₃ catalyst complex (for example, BF₃·MeOH) comprises about 30 wt % or more of BF₃ based on a total wt % of the solid substrate comprising sorbed catalyst complex, the total wt % of the solid substrate comprising sorbed catalyst complex is equal to 100 wt %.

Embodiment B8. The process according to any one of Embodiments B3-B7, wherein: the polymerization unit comprises a tubular loop reactor.

Embodiment B9. The process according to any one of Embodiments B1-B8, wherein the PIB comprises: a first portion comprising polymer chains having alpha vinylidene groups; a second portion comprising polymer chains having beta vinylidene groups; a third portion comprising polymer chains having internal vinylidene groups; the first portion is greater than 75 wt % based on a total wt % of the PIB, the total wt % of the PIB is equal to 100 wt %; and a total of the second portion plus the third portion is 25 wt % or less based on the total wt % of the PIB.

Embodiment B10. The process according to any one of Embodiments B1-B9, wherein the PIB produced comprises a high degree of terminal double bonds (e.g., greater than 75% of polymer chains having alpha vinylidene groups), optionally making it suitable for use in fuel additives, lubricant additives, adhesives, and/or sealants.

Embodiment B11. The process according to any one of Embodiments B1-B10, wherein the process further comprises:
separating isobutylene oligomer coproducts from the polymerization product effluent;
cracking the isobutylene oligomer coproducts into cracked isobutylene; and
optionally, polymerizing the cracked isobutylene into PIB.

Embodiment B12. The process according to Embodiment B11, wherein the cracking the isobutylene oligomer coproducts comprises one or more of:
feeding the isobutylene oligomer coproducts to a cracking reactor;
contacting, under cracking conditions, the isobutylene oligomer coproducts with a cracking catalyst (for example, a zeolite and/or silica-alumina) in the cracking reactor to convert the isobutylene oligomer coproducts into the cracked isobutylene;
operating the cracking reactor under the cracking conditions, the cracking conditions optionally comprising: a temperature in a range from about 400 to about 600° C., such as from about 450 to about 550° C., such as from about 475 to about 525° C., or from about 250 to about 450° C., such as from about 300 to about 400° C.; a pressure of about atmospheric pressure; or a combination thereof; and/or
discharging the isobutylene.

Embodiment B13. The process according to any one of Embodiments B1-B12, wherein the process further comprises: separating isobutylene oligomer coproducts from the polymerization product effluent; and hydrotreating the isobutylene oligomer coproducts to form hydrotreated C8-C20 hydrocarbons.

Embodiment B14. The process according to Embodiment B13, wherein the hydrotreating the isobutylene oligomer coproducts to hydrotreated C8-C20 hydrocarbons comprises one or more of:
feeding the isobutylene oligomer coproducts to a hydrotreatment reactor;
contacting, under hydrotreatment conditions, the isobutylene oligomer coproducts with a hydrotreatment catalyst and hydrogen in the hydrotreatment reactor to convert the isobutylene oligomer coproducts to hydrotreated C8-C20 hydrocarbons, wherein the hydrotreatment conditions optionally comprise: operating the hydrotreatment reactor at a temperature in a range from about 200 to about 350° C.; operating the hydrotreatment reactor at a pressure in a range from about 2 to about 8 MPa; or a combination thereof; and/or
discharging the hydrotreated C8-C20 hydrocarbons.

Embodiment B15. The process according to Embodiment B14, wherein the process further comprises:
feeding the hydrotreated C8-C20 hydrocarbons to an olefin-producing unit;
converting the hydrocarbons to an olefin-containing effluent comprising ethylene in the olefin-producing unit;
optionally separating the ethylene from the olefin-containing effluent; and/or
discharging the ethylene (and optionally using this ethylene for the process for producing the PIB).

Embodiment B16. The process according to any one of Embodiments B1-B15, wherein the process further comprises: hydrogenating the PIB (such as HR-PIB) to form a hydrogenated PIB (such as hydrogenated HR-PIB).

Embodiment B17. The process according to Embodiment B16, wherein hydrogenating the PIB to form the hydrogenated PIB comprises one or more of:
feeding the PIB to a hydrogenation reactor;
contacting, under hydrogenation conditions, the PIB with a hydrogenation catalyst (for example, Ni—Mo, Co—Mo, Pd/C, Pt/C, Ru/C, and/or Raney Ni) in the hydrogenation reactor to convert the PIB into the hydrogenated PIB;
operating the hydrogenation reactor under the hydrogenation conditions, the hydrogenation conditions optionally comprising: a temperature in a range from about 150 to about 350° C., such as from about a temperature in a range from about 150 to about 250° C.; a pressure in a range from about 2 to about 8 MPa, such as from about 3 to about 8 MPa, such as from about 4 to about 6 MPa; or a combination thereof; and/or
discharging the hydrogenated PIB.

Embodiment B18. The process according to any one of Embodiments B16-B17, wherein the hydrogenated PIB comprises a partially unsaturated PIB and/or a fully saturated PIB.

Embodiment C1. A system, comprising:
an olefin-producing unit;
an ethylene dimerization reactor coupled to and downstream from the olefin-producing unit;
a skeletal isomerization reactor coupled to and downstream from the ethylene dimerization reactor;
an isomerization reactor coupled to and downstream from the skeletal isomerization reactor;
a separation unit coupled to and downstream from the isomerization reactor;
a line coupling the separation unit to the skeletal isomerization reactor; and
a controller, the controller coupled to the olefin-producing unit, the ethylene dimerization reactor, the skeletal isomerization reactor, the isomerization reactor, the separation unit, the line, or combinations thereof.

Embodiment C2. The system according to Embodiment C1, wherein the controller is configured to cause the olefin-producing unit: to receive light hydrocarbons (for example, C2-C4 hydrocarbons); to convert the light hydrocarbons to an olefin-containing effluent comprising ethylene; to optionally separate ethylene from the olefin-containing effluent; and/or to discharge the ethylene.

Embodiment C3. The system according to any one of Embodiments C1-C2, wherein the controller is configured to cause the ethylene dimerization reactor: to receive ethylene from the olefin-producing unit; to convert the ethylene to a dimerization product effluent comprising normal butylene; and/or to discharge the dimerization product effluent.

Embodiment C4. The system according to any one of Embodiments C1-C3, wherein the controller is configured to cause the skeletal isomerization reactor: to receive a dimerization product effluent comprising normal butylene from the ethylene dimerization reactor; to convert the normal butylene to a skeletal isomerization product effluent comprising isobutylene; to discharge the skeletal isomerization product effluent.

Embodiment C5. The system according to Embodiment C4, wherein the controller is further configured to cause the skeletal isomerization reactor: to receive 2-butene from the separation unit through the line coupling the separation unit to the skeletal isomerization reactor; and/or to convert the 2-butene to isobutylene.

Embodiment C6. The system according to any one of Embodiments C1-C5, wherein the controller is configured to cause the isomerization reactor: to receive a skeletal isomerization product effluent from the skeletal isomerization reactor; to convert 1-butene present in the skeletal isomerization product effluent to an isomerization product effluent comprising 2-butene; and/or to discharge the isomerization product effluent.

Embodiment C7. The system according to any one of Embodiments C1-C6, wherein the controller is configured to cause the separation unit: to receive an isomerization product effluent from the isomerization reactor; to separate isobutylene from the isomerization product effluent; to separate 2-butene from the isomerization product effluent; to discharge the isobutylene; and/or to discharge the 2-butene through the line coupling the separation unit to the skeletal isomerization reactor.

Embodiment C8. The system according to any one of Embodiments C1-C7, wherein the system further comprises a polymerization unit coupled to and downstream from the separation unit, the controller coupled to the polymerization unit.

Embodiment C9. The system according to Embodiment C8, wherein the controller is coupled to the polymerization unit, and the controller is configured to cause the polymerization unit: to receive isobutylene from the separation unit; to convert the isobutylene received from the separation unit to a polymerization product effluent comprising PIB (for example, HR-PIB); and/or to discharge the PIB.

Embodiment C10. The system according to any one of Embodiments C8-C9, wherein the system further comprises a hydrogenation unit coupled to the polymerization unit.

Embodiment C11. The system according to Embodiment C10, wherein the controller is coupled to the hydrogenation unit, and the controller is configured to cause the hydrogenation unit: to receive at least a portion of the PIB (e.g., HR-PIB) from the polymerization unit; to at least partially hydrogenate the PIB received from the polymerization unit to form a partially unsaturated PIB, a fully saturated PIB, or combinations thereof; and/or to discharge the partially unsaturated PIB, a fully saturated PIB, or combinations thereof.

Embodiment C12. The system according to any one of Embodiments C8-C11, wherein the controller is further configured to cause the polymerization unit to discharge isobutylene oligomer coproducts.

Embodiment C13. The system according to any one of Embodiments C8-C12, wherein the system further comprises a hydrotreatment reactor coupled to and downstream from the polymerization unit.

Embodiment C14. The system according to Embodiment C13, wherein the controller is coupled to the hydrotreatment reactor, and the controller is configured to cause the hydrotreatment reactor: to receive at least a portion of the isobutylene oligomer coproducts from the polymerization unit; to receive hydrogen gas; to convert the isobutylene oligomer coproducts received from the polymerization reactor to an effluent comprising hydrocarbons, the hydrotreated C8-C20 hydrocarbons; and/or to discharge the hydrotreated C8-C20 hydrocarbons.

Embodiment C15. The system according to any one of Embodiments C13-C14, wherein the hydrotreatment reactor is coupled to the olefin-producing unit.

Embodiment C16. The system according to Embodiment C15, wherein the controller is coupled to the olefin-producing unit, and the controller is configured to cause the olefin-producing unit: to receive the hydrotreated C8-C20 hydrocarbons from the hydrotreatment reactor; to convert these hydrotreated C8-C20 hydrocarbons received from the hydrotreatment reactor to ethylene; and/or to discharge the ethylene.

Embodiment C17. The system according to any one of Embodiments C8-C16, wherein the system further comprises a cracking reactor coupled to and downstream from the polymerization unit.

Embodiment C18. The system according to Embodiment C17, wherein the controller is coupled to the cracking reactor, and the controller is configured to cause the cracking reactor: to receive at least a portion of the isobutylene oligomer coproducts from the polymerization unit; to convert the isobutylene oligomer coproducts received from the polymerization unit to isobutylene; and/or to discharge the isobutylene.

Embodiment C19. The system according to any one of Embodiments C17-C18, wherein the controller is configured to cause the polymerization unit to receive the isobutylene discharged from the cracking reactor.

Embodiment C20. The system according to any one of Embodiments C1-C19, wherein the system further comprises a metathesis unit.

Embodiment C21. The system according to Embodiment C20, wherein the controller is coupled to the metathesis unit, and the controller is configured to cause the metathesis unit: to receive 2-butene from the separation unit; to receive ethylene from the olefin-producing unit; to convert the 2-butene received from the separation unit and the ethylene received from the olefin-producing unit to propylene; and/or to discharge the propylene.

Embodiment C22. The system according to any one of Embodiments C1-C21, wherein the controller is configured to perform one or more operations of the process according to any one of Embodiments A1-A18 or B1-B18.

Embodiment D1. A process for producing isobutylene from ethylene and crude C4 (CC4), comprising:
dimerizing ethylene to produce a dimerization product effluent comprising 1-butene, 2-butene, or combinations thereof;
feeding CC4 and the dimerized product effluent into a SHU, wherein the CC4, the dimerized product effluent, or both optionally comprise 1,3-butadiene;
selectively hydrogenating and isomerizing 1,3-butadiene in the SHU to form a selective hydrogenation-isomerization product effluent comprising 2-butene;
separating isobutylene and the 2-butene from the selective hydrogenation-isomerization product effluent;
skeletal isomerizing at least a portion of the 2-butene separated to form a skeletal isomerization product effluent comprising isobutylene;
recycling the skeletal isomerization product effluent to the SHU; and
converting, in the SHU, 1-butene present in the skeletal isomerization product effluent to 2-butene.

Embodiment D2. The process according to Embodiment D1, wherein separating the isobutylene and 2-butene comprises one or more of:
feeding the selective hydrogenation-isomerization product effluent to a separation unit;
operating the separation unit under separation conditions effective to separate the isobutylene and the 2-butene from the selective hydrogenation-isomerization product effluent;
operating the separation unit under separation conditions effective to separate the isobutylene from the 2-butene;
discharging the 2-butene from the separation unit; and/or
discharging the isobutylene from the separation unit.

Embodiment D3. The process according to any one of Embodiments D1-D2, wherein the process further comprises:
(a) converting light hydrocarbons (for example, C2-C6 hydrocarbons) into a first olefin-containing effluent comprising ethylene, CC4, or a combination thereof, wherein optionally at least a portion of the ethylene dimerized to produce the dimerization product effluent is sourced from the first olefin-containing effluent, wherein optionally at least a portion of the CC4 that is fed to the SHU is sourced from the first olefin-containing effluent; and/or
(b) converting isobutylene oligomer coproducts into a second olefin-containing effluent comprising ethylene, CC4, or a combination thereof, wherein optionally at least a portion of the ethylene dimerized to produce the dimerization product effluent is sourced from the second olefin-containing effluent, wherein optionally at least a portion of the CC4 that is fed to the SHU is sourced from the second olefin-containing effluent.

Embodiment D4. The process according to Embodiment D3, wherein at least a portion of the CC4 that is fed to the SHU is sourced, separated, or both, from the first olefin-containing effluent or the second olefin-containing effluent.

Embodiment D5. The process according to any one of Embodiments D3-D4, wherein (a) the converting the light hydrocarbons comprises one or more of:
feeding the light hydrocarbons to an olefin-producing unit;
converting the light hydrocarbons to the first olefin-containing effluent comprising ethylene, CC4, or a combination thereof in the olefin-producing unit;
optionally separating the ethylene from the first olefin-containing effluent;
optionally separating the CC4 from the first olefin-containing effluent;
discharging the ethylene separated; and/or
discharging the CC4 separated.

Embodiment D6. The process according to any one of Embodiments D3-D5, wherein (b) converting isobutylene oligomer coproducts comprises one or more of:
feeding the isobutylene oligomer coproducts to a hydrotreatment reactor;
contacting, under hydrotreatment conditions, the isobutylene oligomer coproducts with a hydrotreatment catalyst and hydrogen in the hydrotreatment reactor to convert the isobutylene oligomer coproducts to hydrotreated C8-C20 hydrocarbons in the hydrotreatment reactor, wherein the hydrotreatment conditions optionally comprise: operating the hydrotreatment reactor at a temperature in a range from about 200 to about 350° C.; and/or operating the hydrotreatment reactor at a pressure in a range from about 0.5 to about 10 MPa;
discharging the hydrotreated C8-C20 hydrocarbons;
feeding the hydrotreated C8-C20 hydrocarbons to an olefin-producing unit (for example, the olefin-producing unit being the same or different from the olefin-producing unit of any one of Embodiments D1-D5);
converting the hydrotreated C8-C20 hydrocarbons to the second olefin-containing effluent comprising ethylene, CC4, or a combination thereof in the olefin-producing unit;
optionally separating the ethylene from the second olefin-containing effluent;
optionally separating the CC4 from the second olefin-containing effluent;
discharging the ethylene separated; and/or
discharging the CC4 separated.

Embodiment D7. The process according to any one of Embodiments D1-D6, wherein dimerizing ethylene to produce the dimerization product effluent comprises one or more of:
feeding the ethylene to an ethylene dimerization reactor;
contacting, under dimerization conditions, the ethylene with a dimerization catalyst (for example, a nickel-based catalyst) in the ethylene dimerization reactor to convert the ethylene to normal butylene;
operating the ethylene dimerization reactor under the dimerization conditions, the dimerization conditions optionally comprising: a temperature in a range from about 50 to about 150° C., such as from about 75 to about 125° C., such as from about 95 to about 105° C.; a pressure in a range from about 1 to about 3 MPa, such as from about 1.5 to about 2.5 MPa, such as from about 1.75 to about 2.25 MPa; or a combination thereof; and/or
discharging the dimerization product effluent comprising normal butylene.

Embodiment D8. The process according to any one of Embodiments D1-D7, wherein selectively hydrogenating and isomerizing the 1,3-butadiene in the SHU to form the selective hydrogenation-isomerization product effluent comprising 2-butene comprises one or more of:
contacting, under selective hydrogenation-isomerization conditions, 1,3-butadiene with a selective hydrogenation-isomerization catalyst (for example a Pd-based catalyst) in the SHU to selectively hydrogenate the 1,3-butadiene into the 1-butene;
contacting, under the selective hydrogenation-isomerization conditions, the 1-butene with the selective hydrogenation-isomerization catalyst in the SHU to isomerize the 1-butene into the 2-butene;
operating the SHU under the selective hydrogenation-isomerization conditions, the selective hydrogenation-isomerization conditions optionally comprising: a temperature in a range from about 50 to about 150° C.; and/or a pressure in a range from about 0.3 to about 4.1 MPa; and/or
discharging a selective hydrogenation-isomerization product effluent comprising the 2-butene; or
combinations thereof.

Embodiment D9. The process according to Embodiment D8, wherein the isomerization of 1-butene to 2-butene during selectively hydrogenating and isomerizing of 1,3-butadiene is performed to facilitate separation of the isobutylene from linear butylenes present in the selective hydrogenation-isomerization product effluent.

Embodiment D10. The process according to any one of Embodiments D1-D9, wherein skeletal isomerizing at least a portion of the 2-butene separated to form the skeletal isomerization product effluent comprising isobutylene comprises one or more of:
feeding the 2-butene separated from the selective hydrogenation-isomerization product effluent to a skeletal isomerization reactor;
contacting, under skeletal isomerization conditions, the 2-butene with an acidic catalyst (for example, silica-alumina) in the skeletal isomerization reactor to convert the 2-butene to isobutylene;
operating the skeletal isomerization reactor under the skeletal isomerization conditions, the skeletal isomerization conditions optionally comprising: a temperature in a range from about 200 to about 500° C., such as from about 200 to about 400° C., such as from about 250 to about 350° C., such as from about 275 to about 325° C., or from about 450 to about 500° C.; and/or a pressure of about atmospheric pressure; or a combination thereof; and/or
discharging the skeletal isomerization product effluent comprising isobutylene, and optionally 2-butene, and optionally 1-butene.

Embodiment D11. The process according to any one of Embodiments D1-D10, wherein the process further comprises:
combining the dimerization product effluent and the selective hydrogenation-isomerization product effluent;
separating isobutylene and 2-butene from the combined dimerization product effluent and the selective hydrogenation-isomerization product effluent; and/or
optionally, one or more of: (i) polymerizing the isobutylene separated into PIB; and/or (ii) skeletal isomerizing the 2-butene separated from the selective hydrogenation-isomerization product effluent to convert the 2-butene separated to isobutylene.

Embodiment D12. The process according to any one of Embodiments D1-D11, wherein the process further comprises:
forming a mixture comprising at least a portion of the 2-butene separated from the selective hydrogenation-isomerization product effluent, and an ethylene stream (the ethylene stream optionally from an olefin-producing unit that is the same or different from the olefin-producing unit of Embodiment D5); and
contacting the mixture with a metathesis catalyst, under metathesis conditions, in a metathesis reactor to form a metathesis product effluent comprising propylene.

Embodiment D13. The process according to Embodiment D12, wherein: the metathesis catalyst comprises a metal oxide of W, Mo, or Re, or a molybdenum alkylidene catalyst, or a rhenium alkylidene catalyst, or combinations thereof; the metathesis conditions comprise a temperature in a range from about 200 to about 350° C., such as from about 250 to about 300° C.; and/or the metathesis conditions comprise a pressure in a range from about 2 to about 5 MPa, such as from about 3 to about 4 MPa.

Embodiment D14. The process according to any one of Embodiments D12-D13, wherein the process further comprises:
(a) removing unreacted 2-butene from the metathesis product effluent; and
(b) one or more of (b1) or (b2):
(b1) recycling unreacted 2-butene removed from the metathesis product effluent back to the metathesis reactor; and/or
(b2) skeletal isomerizing the unreacted 2-butene separated from the metathesis product effluent to form isobutylene.

Embodiment D15. The process according to any one of Embodiments D1-D14, wherein:
at least a portion of the dimerization product effluent bypasses the SHU;
converting 1-butene present in the dimerization product effluent to a 2-butene fraction by use of a reactive distillation column (for example, a reactive distillation column incorporating an acidic isomerization catalyst); and then
separating the 2-butene fraction.

Embodiment D16. The process according to any one of Embodiments D1-D15, wherein the process further comprises:
feeding at least a portion of the dimerization product effluent directly to a separation unit and bypassing the SHU;
converting 1-butene present in the dimerization product effluent to a 2-butene fraction at the separation unit, the separation unit configured as a reactive distillation column; and then
separating the 2-butene fraction at the separation unit.

Embodiment D17. The process according to any one of Embodiments D1-D16, wherein the ethylene dimerized to produce a dimerization product effluent comprising the normal butylene is sourced from: the C2-C6 hydrocarbons converted to the ethylene in the olefin-producing unit; and/or the isobutylene oligomer coproducts converted to the ethylene in the olefin-producing unit.

Embodiment D18. The process according to any one of Embodiments D1-D16, wherein at least a portion of the CC4 that is fed to the SHU is sourced from: the C2-C6 hydrocarbons converted to the CC4 in the olefin-producing unit; the isobutylene oligomer coproducts converted to the CC4 in the olefin-producing unit; n-butane dehydrogenation; n-butane dehydroisomerization; and/or isobutane dehydrogenation.

Embodiment E1. A process for producing PIB (for example, HR-PIB), the process comprising:
(a) a process for producing isobutylene, comprising:
   (a1) dimerizing ethylene to produce a dimerization product effluent comprising 1-butene, 2-butene, or combinations thereof;
   (a2) feeding CC4 and the dimerized product effluent into a SHU, wherein the CC4, the dimerized product effluent, or both optionally comprise 1,3-butadiene;
   (a3) selectively hydrogenating and isomerizing 1,3-butadiene in the SHU to form a selective hydrogenation-isomerization product effluent comprising 2-butene;
   (a4) separating isobutylene and the 2-butene from the selective hydrogenation-isomerization product effluent;
   (a5) skeletal isomerizing at least a portion of the 2-butene separated to form a skeletal isomerization product effluent comprising isobutylene;
   (a6) recycling the skeletal isomerization product effluent to the SHU; and
   (a7) converting, in the SHU, 1-butene present in the skeletal isomerization product effluent to 2-butene; and
(b) polymerizing the isobutylene separated from the selective hydrogenation-isomerization product effluent into a polymerization product effluent comprising PIB (for example, HR-PIB).

Embodiment E2. The process according to Embodiment E1, wherein, after (a6) converting 1-butene present in the skeletal isomerization product effluent to 2-butene in the SHU, the process further comprises: separating isobutylene and the 2-butene from the selective hydrogenation-isomerization product effluent.

Embodiment E3. The process according to any one of Embodiments E1-E2, wherein (a) the process for producing isobutylene comprises any one of Embodiments D1-D18.

Embodiment E4. The process according to any one of Embodiments E1-E3, wherein polymerizing the isobutylene comprises one or more of:
feeding the isobutylene to a polymerization unit;
forming a polymerization reaction mixture comprising the isobutylene and a polymerization catalyst;
reacting the polymerization reaction mixture, under polymerization conditions, in the polymerization unit to convert the isobutylene to the PIB; and/or
discharging the polymerization product effluent comprising the PIB.

Embodiment E5. The process according to Embodiment E4, wherein the polymerization conditions comprise: a temperature in a range from about −10° C. to about 40° C.; a polymerization period of about 4 minutes or less; or a combination thereof.

Embodiment E6. The process according to any one of Embodiments E4-E5, wherein the polymerization catalyst comprises BF₃ (for example, a BF₃ catalyst complex, for example, a solid BF₃ catalyst complex).

Embodiment E7. The process according to any one of Embodiments E4-E6, wherein the polymerization catalyst comprises a BF₃ catalyst complex sorbed on a solid substrate (for example, silica-alumina), the BF₃ catalyst complex comprising: BF₃; and a complexing agent comprising an oxygen-containing compound, the oxygen-containing compound is free of beta-hydrogen atoms.

Embodiment E8. The process according to Embodiment E7, wherein the solid substrate comprising sorbed BF₃ catalyst complex (for example, BF₃·MeOH) comprises about 30 wt % or more of BF₃ based on a total wt % of the solid substrate comprising sorbed catalyst complex, the total wt % of the solid substrate comprising sorbed catalyst complex is equal to 100 wt %.

Embodiment E9. The process according to any one of Embodiments E4-E8, wherein: the polymerization unit comprises a tubular loop reactor.

Embodiment E10. The process according to any one of Embodiments E1-E9, wherein the PIB comprises: a first portion comprising polymer chains having alpha vinylidene groups; a second portion comprising polymer chains having beta vinylidene groups; a third portion comprising polymer chains having internal vinylidene groups; the first portion is greater than 75 wt % based on a total wt % of the PIB, the total wt % of the PIB is equal to 100 wt %; and a total of the second portion plus the third portion is 25 wt % or less based on the total wt % of the PIB.

Embodiment E11. The process according to any one of Embodiments E1-E10, wherein the PIB produced comprises a high degree of terminal double bonds (e.g., greater than 75% of polymer chains having alpha vinylidene groups), optionally making it suitable for use in fuel additives, lubricant additives, adhesives, and/or sealants.

Embodiment E12. The process according to any one of Embodiments E1-E11, wherein the process further comprises:
separating isobutylene oligomer coproducts from the polymerization product effluent;
cracking the isobutylene oligomer coproducts into cracked isobutylene; and
optionally, polymerizing the cracked isobutylene into PIB.

Embodiment E13. The process according to Embodiment E12, wherein the cracking the isobutylene oligomer coproducts comprises one or more of:
feeding the isobutylene oligomer coproducts to a cracking reactor;
contacting, under cracking conditions, the isobutylene oligomer coproducts with a cracking catalyst (for example, a zeolite, silica-alumina, or combinations thereof) in the cracking reactor to convert the isobutylene oligomer coproducts into the cracked isobutylene;
operating the cracking reactor under the cracking conditions, the cracking conditions optionally comprising: a temperature in a range from about 400 to about 600° C., such as from about 450 to about 550° C., such as from about 475 to about 525° C., or from about 250 to about 450° C., such as from about 300 to about 400° C.; a pressure of about atmospheric pressure; or a combination thereof; and/or
discharging the isobutylene.

Embodiment E14. The process according to any one of Embodiments E1-E13, wherein the process further comprises: separating isobutylene oligomer coproducts from the polymerization product effluent; and hydrotreating the isobutylene oligomer coproducts to form hydrotreated C8-C20 hydrocarbons.

Embodiment E15. The process according to Embodiment E14, wherein the hydrotreating the isobutylene oligomer coproducts to form the hydrotreated C8-C20 hydrocarbons comprises one or more of:
feeding the isobutylene oligomer coproducts to a hydrotreatment reactor;
contacting, under hydrotreatment conditions, the isobutylene oligomer coproducts with a hydrotreatment catalyst and hydrogen in the hydrotreatment reactor to convert the isobutylene oligomer coproducts to hydrotreated C8-C20 hydrocarbons in the hydrotreatment reactor, wherein the hydrotreatment conditions optionally comprise: operating the hydrotreatment reactor at a temperature in a range from about 200 to about 350° C.; and/or operating the hydrotreatment reactor at a pressure in a range from about 2 to about 8 MPa;
discharging the hydrotreated C8-C20 hydrocarbons.

Embodiment E16. The process according to Embodiment E1-E15, wherein the process further comprises:
feeding the hydrotreated C8-C20 hydrocarbons to an olefin-producing unit;
converting the hydrocarbons to an olefin-containing effluent comprising ethylene, CC4, or a combination thereof in the olefin-producing unit;
optionally separating the ethylene from the first olefin-containing effluent;
optionally separating the CC4 from the first olefin-containing effluent;
discharging the ethylene separated (and optionally using this ethylene for the process for producing the PIB); and/or
discharging the CC4 separated (and optionally using this ethylene for the process for producing the PIB).

Embodiment E17. The process according to Embodiment E16, wherein: at least a portion of the ethylene dimerized is sourced from the hydrotreated C8-C20 hydrocarbons; and/or at least a portion of the CC4 is sourced from the hydrotreated C8-C20 hydrocarbons.

Embodiment E18. The process according to any one of Embodiments E1-E17, wherein the process further comprises: hydrogenating the PIB (such as HR-PIB) to form a hydrogenated PIB (such as hydrogenated HR-PIB).

Embodiment E19. The process according to Embodiment E18, wherein hydrogenating the PIB to form the hydrogenated PIB comprises one or more of:
feeding the PIB to a hydrogenation reactor;
contacting, under hydrogenation conditions, the PIB with a hydrogenation catalyst (for example, Ni—Mo, Co—Mo, Pd/C, Pt/C, Ru/C, and/or Raney Ni) in the hydrogenation reactor to convert the PIB into the hydrogenated PIB;
operating the hydrogenation reactor under the hydrogenation conditions, the hydrogenation conditions optionally comprising: a temperature in a range from about 150 to about 350° C., such as from about a temperature in a range from about 150 to about 250° C.; a pressure in a range from about 2 to about 8 MPa, such as from about 3 to about 8 MPa, such as from about 4 to about 6 MPa; or a combination thereof; and/or
discharging the hydrogenated PIB.

Embodiment E20. The process according to any one of Embodiments E18-E19, wherein: wherein the hydrogenated PIB comprises a partially unsaturated PIB and/or a fully saturated PIB.

Embodiment F1. A system, comprising:
an olefin-producing unit;
an ethylene dimerization reactor coupled to and downstream from the olefin-producing unit;
a SHU coupled to and downstream from the ethylene dimerization reactor, the SHU further coupled to and downstream from the olefin-producing unit;
a separation unit coupled to and downstream from the SHU;
a skeletal isomerization reactor coupled to and downstream from the separation unit;
a line coupling the skeletal isomerization reactor to the SHU; and
a controller, the controller coupled to the olefin-producing unit, the ethylene dimerization reactor, the SHU, the separation unit, the skeletal isomerization reactor, the line, or combinations thereof.

Embodiment F2. The system according to Embodiment F1, wherein the controller is configured to cause the olefin-producing unit: to receive light hydrocarbons (for example, C2-C4 hydrocarbons); to convert the light hydrocarbons to an olefin-containing effluent comprising ethylene; to optionally separate ethylene from the olefin-containing effluent; and/or to discharge the ethylene.

Embodiment F3. The system according to any one of Embodiments F1-F2, wherein the controller is configured to cause the ethylene dimerization reactor: to receive ethylene from the olefin-producing unit; to convert the ethylene to a dimerization product effluent comprising normal butylene; and/or to discharge the dimerization product effluent.

Embodiment F4. The system according to any one of Embodiments F1-F3, wherein the controller is configured to cause the SHU: to receive dimerization product effluent from the ethylene dimerization reactor; to receive CC4 (optionally from the olefin-producing unit); to convert 1,3-butadiene to 2-butene; and/or to discharge a selective hydrogenation-isomerization product effluent comprising 2-butene (and optionally comprising isobutylene).

Embodiment F5. The system according to any one of Embodiments F1-F4, wherein the controller is configured to cause the separation unit to: to receive a selective hydrogenation-isomerization product effluent from the SHU; to separate an isobutylene fraction from the selective hydrogenation-isomerization product effluent; to separate a 2-butene fraction from the selective hydrogenation-isomerization product effluent; to discharge the isobutylene fraction; and/or to discharge the 2-butene fraction.

Embodiment F6. The system according to any one of Embodiments F1-F5, wherein the controller is configured to cause the skeletal isomerization reactor: to receive a 2-butene fraction from the separation unit; to convert the 2-butene fraction to a skeletal isomerization product effluent comprising isobutylene; and/or to discharge the skeletal isomerization product effluent through the line coupling the skeletal isomerization reactor to the SHU.

Embodiment F7. The system according to any one of Embodiments F1-F6, wherein the controller is further configured to cause the SHU: to receive a skeletal isomerization product effluent from the skeletal isomerization reactor by the line coupling the skeletal isomerization reactor to the SHU; to convert 1-butene present in the skeletal isomerization reactor product effluent to 2-butene; and/or to discharge the selective hydrogenation-isomerization product effluent comprising 2-butene and isobutylene.

Embodiment F8. The system according to any one of Embodiments F1-F7, wherein the system further comprises a polymerization unit coupled to and downstream from the separation unit.

Embodiment F9. The system according to Embodiment F8, wherein the controller is coupled to the polymerization unit, and the controller is configured to cause the polymerization unit: to receive isobutylene from the separation unit; to convert the isobutylene received from the separation unit to a polymerization product effluent comprising PIB (for example, HR-PIB); and/or to discharge the PIB (for example, HR-PIB).

Embodiment F10. The system according to any one of Embodiments F8-F9, wherein the system further comprises a hydrogenation unit coupled to the polymerization unit.

Embodiment F11. The system according to Embodiment F10, wherein the controller is coupled to the hydrogenation unit, and the controller is configured to cause hydrogenation unit: to receive at least a portion of the HR-PIB from the polymerization unit; at least partially hydrogenate the HR-PIB received from the polymerization unit to form a partially unsaturated HR-PIB, a fully saturated HR-PIB, or combinations thereof; and/or to discharge the partially unsaturated HR-PIB, a fully saturated HR-PIB, or combinations thereof.

Embodiment F12. The system according to any one of Embodiments F8-F11, wherein the controller is further configured to cause the polymerization unit to discharge isobutylene oligomer coproducts.

Embodiment F13. The system according to any one of Embodiments F8-F12, wherein the system further comprises a hydrotreatment reactor coupled to and downstream from the polymerization unit.

Embodiment F14. The system according to Embodiment F13, wherein the controller is coupled to the hydrotreatment reactor, and the controller is configured to cause the hydrotreatment reactor: to receive at least a portion of the isobutylene oligomer coproducts from the polymerization unit; to receive hydrogen gas; to convert the isobutylene oligomer coproducts received from the polymerization reactor to an effluent comprising hydrocarbons, the hydrotreated C8-C20 hydrocarbons; and/or to discharge the hydrotreated C8-C20 hydrocarbons.

Embodiment F15. The system according to any one of Embodiments F13-F14, wherein the hydrotreatment reactor is coupled to the olefin-producing unit.

Embodiment F16. The system according to Embodiment F15, wherein the controller is configured to cause the olefin-producing unit: to receive the hydrotreated C8-C20 hydrocarbons from the hydrotreatment reactor; to convert these hydrotreated C8-C20 hydrocarbons received from the hydrotreatment reactor to ethylene; and/or to discharge the ethylene.

Embodiment F17. The system according to any one of Embodiments F8-F16, wherein the system further comprises a cracking reactor coupled to and downstream from the polymerization unit.

Embodiment F18. The system according to Embodiment F17, wherein the controller is coupled to the cracking reactor, and the controller is configured to cause the cracking reactor: to receive at least a portion of the isobutylene oligomer coproducts from the polymerization unit; to convert the isobutylene oligomer coproducts received from the polymerization unit to isobutylene; and/or to discharge the isobutylene.

Embodiment F19. The system according to any one of Embodiments F17-F19, wherein the polymerization unit is further configured to receive the isobutylene discharged from the cracking reactor.

Embodiment F20. The system according to any one of Embodiments F1-F19, wherein the system further comprises a metathesis unit.

Embodiment F21. The system according to Embodiment F20, wherein the controller is coupled to the metathesis unit, and the controller is configured to cause the metathesis unit: to receive 2-butene from the separation unit; to receive ethylene from the olefin-producing unit; to convert the 2-butene (received from the separation unit) and the ethylene (received from the olefin-producing unit) to propylene; and/or to discharge the propylene.

Embodiment F22. The system according to any one of Embodiments F1-F21, wherein the controller is configured to perform one or more operations of the process according to any one of Embodiments D1-D18 or E1-E20.

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2nd Ed (1997), may be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

“Contacting” and “combining” may be used herein to describe systems, compositions, methods, and processes in which the materials or components are contacted or combined together in any suitable order, in any suitable manner, and for any suitable length of time, unless otherwise specified. For example, the materials or components may be blended, mixed, slurried, dissolved, reacted, treated, impregnated, compounded, or otherwise contacted or combined in some other suitable manner or by any suitable method or technique.

Generally, groups of elements are indicated using the numbering scheme indicated in the version of the periodic table of elements published in Chemical and Engineering News, 63(5), 27, 1985. In some instances, a group of elements may be indicated using a common name assigned to the group; for example, alkali metals for Group 1 elements, alkaline earth metals for Group 2 elements, transition metals for Group 3-12 elements, and halogens or halides for Group 17 elements.

The term “olefin” is used herein in accordance with the definition specified by IUPAC: acyclic and cyclic hydrocarbons having one or more carbon-carbon double bonds apart from the formal ones in aromatic compounds.

In the foregoing, reference is made to embodiments of the disclosure. However, it should be understood that the disclosure is not limited to specific described embodiments. Instead, any combination of the features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the foregoing embodiments, features, aspects, implementations, and advantages are merely illustrative and are not considered elements or limitations of the appended claims except where explicitly recited in a claim(s). Likewise, reference to “the disclosure” shall not be construed as a generalization of any inventive subject matter described herein and shall not be considered to be an element or limitation of the appended claims except where explicitly recited in a claim(s).

As is apparent from the foregoing general description and the specific embodiments, while forms of the embodiments have been illustrated and described, various modifications may be made without departing from the spirit and scope of the present disclosure. Accordingly, it is not intended that the present disclosure be limited thereby. Likewise, the term “comprising” is considered synonymous with the term “including.” Likewise whenever a formulation, a composition, an element or a group of elements is preceded with the transitional phrase “comprising,” it is understood that we also contemplate the same formulation, composition or group of elements with transitional phrases “consisting essentially of,” “consisting of,” “selected from the group of consisting of,” or “Is” preceding the recitation of the formulation, composition, element, or elements and vice versa, for example, the terms “comprising,” “consisting essentially of,” “consisting of” also include the product of the combinations of elements listed after the term.

References cited herein are incorporated by reference herein in their entirety to indicate the state of the art as of their publication or filing date and it is intended that this information may be employed herein, if desired, to exclude specific aspects that are in the prior art.

For purposes of this present disclosure, and unless otherwise specified, all numerical values within the detailed description and the claims herein are modified by “about” or “approximately” the indicated value, and consider experimental error and variations that would be expected by a person having ordinary skill in the art. It will be further understood that there are a number of values disclosed therein, and that each value is also herein disclosed as “about” that particular value in addition to the value itself. In embodiments, use of the term “about” may refer to ±20% of the stated value, ±15% of the stated value, ±10% of the stated value, ±5% of the stated value, ±3% of the stated value, ±2% of the stated value, or ±1% of the stated value.

For the sake of brevity, only certain ranges are explicitly disclosed herein. However, ranges from any lower limit may be combined with any upper limit to recite a range not explicitly recited, as well as ranges from any lower limit may be combined with any other lower limit to recite a range not explicitly recited, in the same way, ranges from any upper limit may be combined with any other upper limit to recite a range not explicitly recited. Additionally, within a range includes every point or individual value between its end points even though not explicitly recited. Thus, every point or individual value may serve as its own lower or upper limit combined with any other point or individual value or any other lower or upper limit, to recite a range not explicitly recited. For example, by disclosing a temperature of from 70° C. to 80° C., an intent is to recite individually 70° C., 71° C., 72° C., 73° C., 74° C., 75° C., 76° C., 77° C., 78° C., 79° C., and 80° C., including any sub-ranges and combinations of sub-ranges encompassed therein such that any of the foregoing numbers may be used singly to describe an open-ended range or in combination to describe a close-ended range. Moreover, all numerical end points of ranges disclosed herein are approximate, unless excluded by proviso. As a representative example, if one or more operations in the processes described herein may be conducted at a temperature in a range from 10° C. to 75° C., this range should be interpreted as encompassing temperatures in a range from “about” 10° C. to “about” 75° C. As another example, when a chemical moiety having a certain number of carbon atoms is disclosed or claimed, the intent is to disclose or claim individually every possible number that such a range could encompass, consistent with the disclosure herein. For example, the disclosure that a moiety is a C1 to C18 hydrocarbyl group, or in alternative language, a hydrocarbyl group having from 1 to 18 carbon atoms, refers to a moiety that may have 1, 2, 3, 4, 5, 6, 7, 8, 9, 10, 11, 12, 13, 14, 15, 16, 17, or 18 carbon atoms, as well as any range between these two numbers (for example, a C1 to C8 hydrocarbyl group), and also including any combination of ranges between these two numbers (for example, a C2-C4 and a C12-C16 hydrocarbyl group).

As used herein, the indefinite article “a” or “an” shall mean “at least one” unless specified to the contrary or the context clearly indicates otherwise. For example, embodiments comprising “a polymerization catalyst” include embodiments comprising one, two, or more polymerization catalysts, unless specified to the contrary or the context clearly indicates only one polymerization catalyst is included.

While the foregoing is directed to embodiments of the present disclosure, other and further embodiments of the disclosure may be devised without departing from the basic scope thereof, and the scope thereof is determined by the claims that follow.

What is claimed is:

1. A process for producing isobutylene, the process comprising:
dimerizing ethylene to produce a dimerization product effluent comprising a normal butylene;
skeletal isomerizing at least a portion of the normal butylene present in the dimerization product effluent to form a skeletal isomerization product effluent comprising isobutylene;
isomerizing at least a portion of 1-butene present in the skeletal isomerization product effluent to form an isomerization product effluent comprising 2-butene; and
separating isobutylene and 2-butene from the isomerization product effluent.

2. The process according to claim 1, wherein, after separating the isobutylene and the 2-butene from the isomerization product effluent, the process further comprises:
skeletal isomerizing the 2-butene separated from the isomerization product effluent to convert the 2-butene separated to isobutylene.

3. The process according to claim 1, wherein the process further comprises:
(a) converting C2-C6 hydrocarbons into a first olefin-containing effluent comprising ethylene;
(b) converting isobutylene oligomer coproducts into a second olefin-containing effluent comprising ethylene; or
both (a) and (b).

4. The process according to claim 3, wherein at least a portion of the ethylene dimerized to produce the dimerization product effluent is sourced from:
the first olefin-containing effluent;
the second olefin-containing effluent; or
combinations thereof.

5. The process according to claim 3, wherein the isobutylene oligomer coproducts are formed by:
polymerizing the isobutylene separated from the isomerization product effluent into a polymerization product effluent, the polymerization product effluent comprising the isobutylene oligomer coproducts.

6. The process according to claim 5, wherein:
the polymerization product effluent further comprises polyisobutylene; and
the process further comprises hydrogenating the polyisobutylene to form hydrogenated polyisobutylene, the hydrogenated polyisobutylene comprising:
a partially unsaturated polyisobutylene;
a fully saturated polyisobutylene; or
combinations thereof.

7. The process according to claim 1, wherein the process further comprises:
forming a mixture comprising at least a portion of the 2-butene separated from the isomerization product effluent and an ethylene stream; and
contacting the mixture with a metathesis catalyst, under metathesis conditions, to form a metathesis product effluent comprising propylene.

8. A process for producing isobutylene from ethylene and crude C4 (CC4), comprising:
dimerizing ethylene to produce a dimerization product effluent comprising 1-butene, 2-butene, or combinations thereof;
feeding CC4 and the dimerized product effluent into a selective hydrogenation-isomerization unit (SHU), wherein the CC4 comprises 1,3-butadiene;
selectively hydrogenating and isomerizing 1,3-butadiene in the SHU to form a selective hydrogenation-isomerization product effluent comprising 2-butene;
separating isobutylene and the 2-butene from the selective hydrogenation-isomerization product effluent;
skeletal isomerizing at least a portion of the 2-butene separated to form a skeletal isomerization product effluent comprising isobutylene;
recycling the skeletal isomerization product effluent to the SHU; and
converting, in the SHU, 1-butene present in the skeletal isomerization product effluent to 2-butene.

9. The process according to claim 8, wherein the process further comprises:
(a) converting C2-C6 hydrocarbons into a first olefin-containing effluent comprising ethylene, CC4, or a combination thereof;
(b) converting isobutylene oligomer coproducts into a second olefin-containing effluent comprising ethylene, CC4, or a combination thereof; or
both (a) and (b).

10. The process according to claim 9, wherein at least a portion of the ethylene dimerized to produce the dimerization product effluent is sourced from:
the first olefin-containing effluent;
the second olefin-containing effluent; or
combinations thereof.

11. The process according to claim 9, wherein at least a portion of the CC4 that is fed to the SHU is sourced from:
the first olefin-containing effluent;
the second olefin-containing effluent;
n-butane dehydrogenation;
n-butane dehydroisomerization;
isobutane dehydrogenation; or
combinations thereof.

12. The process according to claim 8, wherein the process further comprises:
forming a mixture comprising at least a portion of the 2-butene separated from the selective hydrogenation-isomerization product effluent and an ethylene stream; and
contacting the mixture with a metathesis catalyst, under metathesis conditions, to form a metathesis product effluent comprising propylene.

13. The process according to claim 8, wherein the process further comprises:
feeding at least a portion of the dimerization product effluent directly to a separation unit and bypassing the SHU;
converting 1-butene present in the dimerization product effluent to a 2-butene fraction at the separation unit, the separation unit configured as a reactive distillation column; and then
separating the 2-butene fraction at the separation unit.

14. A process for producing polyisobutylene, the process comprising:
dimerizing ethylene to produce a dimerization product effluent comprising 1-butene, 2-butene, or combinations thereof;
feeding CC4 and the dimerized product effluent into a selective hydrogenation-isomerization unit (SHU), wherein the CC4 comprises 1,3-butadiene;
selectively hydrogenating and isomerizing 1,3-butadiene in the SHU to form a selective hydrogenation-isomerization product effluent comprising 2-butene;
separating isobutylene and the 2-butene from the selective hydrogenation-isomerization product effluent;
skeletal isomerizing at least a portion of the 2-butene separated to form a skeletal isomerization product effluent comprising isobutylene;
recycling the skeletal isomerization product effluent to the SHU;
converting, in the SHU, 1-butene present in the skeletal isomerization product effluent to 2-butene; and
polymerizing the isobutylene separated from the selective hydrogenation-isomerization product effluent into a polymerization product effluent comprising polyisobutylene.

15. The process according to claim 14, wherein the polyisobutylene comprises:
a first portion comprising polymer chains having alpha vinylidene groups;
a second portion comprising polymer chains having beta vinylidene groups;
a third portion comprising polymer chains having internal vinylidene groups;
the first portion is greater than 75 wt % based on a total wt % of the polyisobutylene, the total wt % of the polyisobutylene is equal to 100 wt %; and
a total of the second portion plus the third portion is 25 wt % or less based on the total wt % of the polyisobutylene.

16. The process according to claim 14, wherein the process further comprises:
separating isobutylene oligomer coproducts from the polymerization product effluent;
cracking the isobutylene oligomer coproducts into cracked isobutylene; and
polymerizing the cracked isobutylene into polyisobutylene.

17. The process according to claim 14, wherein the process further comprises:
separating isobutylene oligomer coproducts from the polymerization product effluent; and
hydrotreating the isobutylene oligomer coproducts to form hydrotreated C8-C20 hydrocarbons.

18. The process according to claim 17, wherein at least a portion of the ethylene dimerized is sourced from:
the hydrotreated C8-C20 hydrocarbons;
steam cracking of C2-C6 hydrocarbons; or
a combination thereof.

19. The process according to claim 17, wherein at least a portion of the CC4 is sourced from:
the hydrotreated C8-C20 hydrocarbons;
n-butane dehydrogenation;
n-butane dehydroisomerization;
isobutane dehydrogenation; or
combinations thereof.

20. The process according to claim 14, wherein the process further comprises hydrogenating the polyisobutylene to form hydrogenated polyisobutylene, the hydrogenated polyisobutylene comprising a partially unsaturated polyisobutylene, a fully saturated polyisobutylene, or combinations thereof.
